(19) United States
(12) Patent Application Publication
(10) Pub. No.: US 2025/0250283 A1
(43) Pub. Date: Aug. 7, 2025

(54) COMPOUND CAPABLE OF REGULATING AND CONTROLLING ACTIVITY OF 15-PGDH, AND PREPARATION METHOD THEREFOR

(71) Applicant: SCINNOHUB PHARMACEUTICAL CO., LTD., Chengdu, Sichuan Province (CN)

(72) Inventors: Sen JI, Chengdu, Sichuan Province (CN); Hao WANG, Chengdu, Sichuan Province (CN); Wenqing ZHANG, Chengdu, Sichuan Province (CN); Jie LIANG, Chengdu, Sichuan Province (CN); Xiao WANG, Chengdu, Sichuan Province (CN); Lin TIAN, Chengdu, Sichuan Province (CN); Ze FENG, Chengdu, Sichuan Province (CN); Nan LI, Chengdu, Sichuan Province (CN); Yuangqing TANG, Chengdu, Sichuan Province (CN); Jing WANG, Chengdu, Sichuan Province (CN); Xiaodong ZHANG, Chengdu, Sichuan Province (CN); Jun TANG, Chengdu, Sichuan Province (CN)

(21) Appl. No.: 18/856,225

(22) PCT Filed: Apr. 13, 2023
(86) PCT No.: PCT/CN2023/088006

§ 371 Date: Oct. 11, 2024

(30) Foreign Application Priority Data
Apr. 13, 2022 (CN) 202210385527.5
Mar. 21, 2023 (CN) 202310279357.7

(51) Int. Cl. C07D 495/14; A61K 31/519

(52) U.S. Cl. CPC C07D 495/14; A61K 31/519

(57) ABSTRACT

Disclosed are a compound of formula (I) capable of regulating and controlling the activity of 15-PGDH, or a stereoisomer thereof, a tautomer thereof or a mixture thereof, or a pharmaceutically acceptable salt thereof, or a solvate thereof, or a prodrug thereof, a pharmaceutical composition comprising the same, and use of the compound in the preparation of a 15-PGDH inhibitor.

### TECHNICAL FIELD

The present application relates to a compound for regulating the activity of 15-PGDH and a preparation method thereof, specifically relates to a compound that can be used as a medicament for regulating the activity of 15-PGDH, and a pharmaceutically acceptable salt thereof, a composition comprising the compound or the salt thereof, and use thereof in the preparation of a medicament, belonging to the field of pharmaceutical chemistry.

### BACKGROUND

15-Hydroxyprostaglandin dehydrogenase (15-PGDH) belongs to an evolutionarily conserved superfamily of short-chain dehydrogenases/reductases (SDRs), and has been designated as SDR36C1 according to the recently approved nomenclature for human enzymes. Based on current research results, the majority of the in vivo activity can be attributed to HPGD gene-encoded type I 15-PGDH. 15-PGDH plays an important role in the inactivation of active prostaglandins (PGD2, PGE1, PGE2, PGF2α, PGI2, etc.), hydroxyeicosatetraenoic acid (HETEs), and inflammation-resolving lipid mediators (RvD1, RvD2, RvE1, MaR1, LXA4, etc.) (hereinafter referred to generally as 15-PGDH substrates) (e.g., by catalyzing the oxidation reaction of the hydroxyl group at position 15 of PGF2α into 15-keto-PGF2α). These 15-PGDH substrates exert their functions through specific receptors on target cells. Among others, prostaglandins PGE1, PGE2, PGF2α, PGI2, etc. are often used to assess 15-PGDH activity. For example, PGDH activity is assessed by measuring ketone metabolites of the 15-position hydroxyl group of PGF2α (Journal of Clinical Endocrinology and Metabolism, Vol 84, No. 1, 291-299).

Receptors of 15-PGDH substrates are widely and differentially distributed in vivo, and the diversity of expression distributions, receptor types, and signaling together create a diversity of functions in vivo. For example, PGE1 acts on blood vessels and platelets to promote an increase in blood flow by vasodilatory effects and inhibition of platelet aggregation, and is therefore commonly used for treating diseases such as chronic arterial occlusion (thromboangiitis obliterans (TAO) or occlusive arteriosclerosis obliterans (ASO)), skin ulcers; PGF2α has uterine constraction and intraocular pressure lowering effects, and derivatives thereof have been used as therapeutic agents for glaucoma; and PGD2 inhibits inflammation by enhancing the barrier function of the pulmonary blood vessels. In addition, PGE2 has vasodilatory effects, and also a variety of effects involving blood pressure, pain, bone formation and cell growth, stem cell differentiation, and anti-fibrotic and anti-inflammatory effects, etc. PGI2 has an inhibitory effect on platelet activation and a relaxing effect on vascular smooth muscle, and its derivatives are used as therapeutic agents for chronic arterial occlusion and primary pulmonary hypertension. Inflammation-resolving lipid mediators (RvD1, RvD2, RvE1, MaR1, LXA4, etc.) inhibit migration/activation of neutrophils and accelerate apoptosis of neutrophils. In addition, it is indispensable in the process of increasing the phagocytic activity of macrophages to effectively remove apoptotic neutrophils/tissue debris remaining at the site of inflammation. These functions can promote inflammation and maintain homeostasis within the organism. These inflammation-resolving lipid mediators have been reported to show medicinal efficacy in various types of pathology models (e.g., mouse pulmonary inflammation model, colitis model, and liver injury model).

Recent studies indicate that 15-PGDH inhibitors and 15-PGDH agonists may have therapeutic values. A recent study indicates that the expression of 15-PGDH in protection against thrombin-mediated cell death is increased. It is well known that the 15-PGDH causes the inactivation of prostaglandin E2 (PGE2), and the prostaglandin E2 is a downstream product of COX-2 metabolism. Studies have shown that PGE2 is beneficial in a variety of biological processes, such as maintaining hair density, promoting skin wound healing and bone formation.

15-PGDH as an important enzyme in the inactivation of 15-PGDH substrates involves a wide range of in vivo roles, and 15-PGDH inhibitors may be used to prevent or treat diseases associated with 15-PGDH and/or 15-PGDH substrates and/or when there is a need to increase the level of 15-PGDH substrates in the body of a subject.

As mentioned above, some substrates of 15-PGDH have anti-fibrotic, anti-inflammatory, blood flow improvement, growth-promoting, stem cell increase-promoting, smooth muscle contraction/relaxation-promoting, immunosuppression and bone metabolism-affecting effects, etc. Thus, 15-PGDH inhibitors may effectively treat or prevent fibrosis (e.g., pulmonary fibrosis (idiopathic pulmonary fibrosis, etc.), hepatic fibrosis, renal fibrosis, myocardial fibrosis, scleroderma, and myelofibrosis), inflammatory diseases (e.g., chronic obstructive pulmonary disease (COPD), acute lung injuries, sepsis, lung disease and asthma, inflammatory bowel disease (e.g., ulcerative colitis and Crohn's disease), peptic ulcers (e.g., NSAID-induced ulcers), autoinflammatory diseases (e.g., Behçet's disease), vasculitis syndromes, acute liver injury, acute kidney injury, nonalcoholic steatohepatitis (NASH), atopic dermatitis, psoriasis, interstitial cystitis, prostatitis syndrome (e.g., chronic prostatitis/chronic pelvic pain syndrome)), and cardiovascular disease (e.g., pulmonary arterial hypertension, angina pectoris, myocardial infarction, heart failure, ischemic heart disease, chronic kidney disease, renal failure, cerebral apoplexy, and peripheral circulatory disorders), trauma (e.g., diabetic ulcers, burns, pressure ulcers, acute mucosal injuries (including Stevens-Johnson syndrome; and mucosal injuries associated with alkylating agents, inhibitors of DNA synthesis, inhibitors of DNA gyrase, antimetabolites and other anticancer chemotherapeutic agents, mucosal injuries associated with cellular humoral immunotherapy, mucosal injuries associated with graft-versus-host disease, such as mucositis or stomatitis)), autoimmune diseases (e.g., multiple sclerosis or rheumatoid arthritis), graft-versus-host disease (GVHD), hair growth disorder, osteoporosis, ear diseases (e.g., hearing loss, tinnitus, vertigo, and dysequilibrium), eye diseases (e.g., glaucoma and dry eye), diabetes mellitus, underactive bladder, neutropenia, neurological diseases caused by transplantation of stem cells, bone marrows or organs (e.g., psychoneurosis, neuropathies, neurotoxic diseases, neuropathic pain, and neurodegenerative diseases), and muscle regenerative diseases (e.g., muscular dystrophy, myodystrophy, and muscle injuries); in addition, 15-PGDH inhibitors may also be used to promote cervical ripening.

Compounds and pharmaceutically acceptable salts thereof provided in the present application further meet the requirement of small molecules for inhibiting the activity of 15-PGDH.

### SUMMARY OF THE INVENTION

The present application provides a compound represented by formula (I), a stereoisomer, tautomer or mixture form thereof, or a pharmaceutically acceptable salt thereof, or a solvate (such as a hydrate) thereof, or a prodrug thereof:

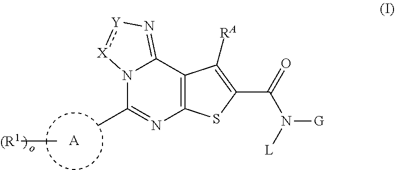

ring A is selected from an aromatic ring, an aromatic heterocycle, and an unsaturated aliphatic heterocycle; L and G are each independently selected from a C₁-C₁₀ chain hydrocarbon group, or L, G, and an N atom connected thereto together form a cyclic group, and the cyclic group is a 3- to 12-membered saturated aliphatic heterocycle or a ring formed by a 3- to 12-membered saturated aliphatic heterocycle fused with a benzene ring; o is selected from 0, 1, 2, and 3;
R¹ is each independently selected from deuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₂-C₆ alkenyl, C₁-C₆ alkoxy, and a 3- to 8-membered saturated aliphatic heterocycle, or when o is selected from 2 and 3, any two R¹ and atoms of ring A connected thereto together form a 3- to 8-membered alicyclic group or a 3- to 8-membered aliphatic heterocyclic group; [symbol] is a single bond or a double bond, and when [symbol] is a double bond, X and Y are each independently selected from CR^{B} and N; when [symbol] is a single bond, X and Y are CR^{C}R^{D};
R^{A}, R^{B}, R^{C}, and R^{D} are each independently selected from hydrogen, hydroxyl, halogen, an amine group, cyano, C₃-C₈ cycloalkyl, C₁-C₆ alkyl, C₁-C₆ alkoxy, and C₁-C₆ haloalkyl;
wherein the aromatic heterocycle, the saturated aliphatic heterocycle, the unsaturated aliphatic heterocycle, and the aliphatic heterocyclic group each independently include 1-3 heteroatoms which are independently selected from N, O, and S, and the saturated aliphatic heterocycle includes at least 1 nitrogen atom;
the L, G, and R¹ are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6- to 10-membered aryl, and 5- to 10-membered heteroaryl.

Further, in certain embodiments of the present application, the L and G are each independently selected from C₁-C₁₀ alkyl, and the L and G are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6- to 10-membered aryl, and 5- to 10-membered heteroaryl;
preferably, the L and G are each independently selected from methyl, ethyl, n-propyl, isopropyl, n-butyl, isobutyl, tert-butyl, hydroxymethyl, hydroxyethyl, and hydroxypropyl.

In some embodiments, ring A is selected from an aromatic ring, an aromatic heterocycle, and an unsaturated aliphatic heterocycle;
L and G are each independently selected from a C₁-C₁₀ chain hydrocarbon group, or L, G, and an N atom connected thereto together form a cyclic group, and the cyclic group is a 3- to 12-membered saturated aliphatic heterocycle or a ring formed by a 3- to 12-membered saturated aliphatic heterocycle fused with a benzene ring;
is selected from 0, 1, 2, and 3;
R¹ is each independently selected from deuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₁-C₆ alkoxy, and a 3- to 8-membered saturated aliphatic heterocycle, or when o is selected from 2 and 3, any two R¹ and atoms of ring A connected thereto together form a 3- to 8-membered alicyclic group or a 3- to 8-membered aliphatic heterocyclic group; [symbol] is a single bond or a double bond, and when [symbol] is a double bond, X and Y are each independently selected from CR^{B} and N; when [symbol] is a single bond, X and Y are CR^{C}R^{D},
R^{A}, R^{B}, R^{C}, and R^{D} are each independently selected from hydrogen, hydroxyl, halogen, an amine group, cyano, C₃-C₈ cycloalkyl, C₁-C₆ alkyl, C₁-C₆ alkoxy, and C₁-C₆ haloalkyl;
wherein the aromatic heterocycle, the saturated aliphatic heterocycle, the unsaturated aliphatic heterocycle, and the aliphatic heterocyclic group each independently include 1-3 heteroatoms which are independently selected from N, O, and S, and the saturated aliphatic heterocycle includes at least 1 nitrogen atom;
the L, G, and R¹ are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6- to 10-membered aryl, and 5- to 10-membered heteroaryl.

In certain embodiments of the present application, the [symbol] is a single bond, the X and Y are CR^{C}R^{D}, and the R^{C} and the R^{D} are selected from hydrogen, hydroxyl, cyano, halogen, methyl, ethyl, n-propyl, isopropyl, methoxy, ethoxy, trifluoromethyl, trifluoroethyl, trichloromethyl, trichloroethyl, cyclopropyl, cyclobutyl, and cyclopentyl.

In certain embodiments of the present application, the [symbol] is a double bond, at least one of the X and Y is selected from CR^{B}, and the R^{B} is selected from hydrogen, hydroxyl, cyano, halogen, C₃-C₈ cycloalkyl, C₁-C₆ alkyl, C₁-C₆ alkoxy, and C₁-C₆ haloalkyl; preferably, the R^{B} is selected from hydrogen, hydroxyl, cyano, halogen, methyl, ethyl, n-propyl, isopropyl, methoxy, ethoxy, trifluoromethyl, trifluoroethyl, trichloromethyl, trichloroethyl, cyclopropyl, cyclobutyl, and cyclopentyl.

In certain embodiments of the present application, the R^{A} is selected from hydrogen, hydroxyl, cyano, fluoro, chloro, bromo, —NH₂, methyl, ethyl, isopropyl, methoxy, ethoxy, trifluoromethyl, trifluoroethyl, trichloromethyl, trichloroethyl, cyclopropyl, cyclobutyl, and cyclopentyl.

In certain embodiments of the present application, the ring A is selected from a 6- to 10-membered aromatic ring, a 5- to 10-membered aromatic heterocycle, and a 6- to 8-membered unsaturated aliphatic heterocycle; preferably, the aromatic ring and the aromatic heterocycle are a monocycle or a fused ring, the unsaturated aliphatic heterocycle is a monocycle, the aromatic heterocycle and the unsaturated aliphatic heterocycle each independently include 1-3 heteroatoms, and the heteroatom is independently selected from N, O, and S.

In certain embodiments of the present application, the ring A is selected from a 6- to 10-membered aromatic ring, and a 5- to 10-membered aromatic heterocycle containing 1-3 heteroatoms (preferably 1-2 heteroatoms), and the heteroatom is independently selected from N, O, and S. In some preferred embodiments, the ring A is a benzene ring, or a 5- to 10-membered aromatic heterocycle containing 1-2 heteroatoms, and the heteroatom is independently N or S.

In certain embodiments of the present application, o is 0 or 2, and when o is 2, any two R¹ and atoms of ring A connected thereto together form a 3- to 8-membered (such as 5- to 7-membered or 5- to 6-membered) aliphatic heterocyclic group. In some preferred embodiments, o is 0 or 2, when o is 2, any two R¹ and atoms of ring A connected thereto together form a 5- to 7-membered (preferably 5- to 6-membered) aliphatic heterocyclic group containing 1-3 heteroatoms (preferably 1-2 heteroatoms, or 1 heteroatom), and the heteroatom is independently N, O, or S (preferably N). In some preferred embodiments, the R¹ is optionally substituted by one or two independently selected from ═O, or C₁-C₆ alkyl (preferably C₁-C₅ alkyl, C₁-C₄ alkyl, or C₁-C₃ alkyl). In some preferred embodiments, o is 0 or 2, when o is 2, any two R¹ and atoms of ring A connected thereto together form saturated or unsaturated 5- to 6-membered cycloamido, and the cycloamido is unsubstituted or further substituted by C₁-C₆ alkyl (preferably C₁-C₅ alkyl, C₁-C₄ alkyl, or C₁-C₃ alkyl). In some preferred embodiments, o is 0 or 2, when o is 2, any two R¹ and atoms of ring A connected thereto together form saturated or unsaturated 5- to 6-membered cycloamido, and the cycloamido is optionally substituted by C₁-C₆ alkyl (preferably C₁-C₅ alkyl, C₁-C₄ alkyl, or C₁-C₃ alkyl) at the N atom therein.

In certain embodiments of the present application, the L and G are each independently selected from C₁-C₆ alkyl (preferably C₁-C₅ alkyl, C₁-C₄ alkyl, or C₁-C₃ alkyl), or the L, G, and an N atom connected thereto together form a 5- to 7-membered (preferably 5- to 6-membered) saturated aliphatic heterocycle containing 1-3 heteroatoms (preferably 1-2 heteroatoms), and the heteroatom is independently N, O, or S (preferably N). In some preferred embodiments, the L and G are each independently selected from C₁-C₅ alkyl (preferably C₁-C₄ alkyl, or C₁-C₃ alkyl), or the L, G, and an N atom connected thereto together form a 5- to 6-membered saturated aliphatic heterocycle containing 1-2 heteroatoms (such as 1 heteroatom), the heteroatom is N, and the saturated aliphatic heterocycle is optionally substituted by halogen (such as fluorine, chlorine, bromine, or iodine) or C₁-C₆ alkyl (preferably, the saturated aliphatic heterocycle is substituted by fluorine, chlorine, bromine, or iodine).

In certain embodiments of the present application, the L and G are optionally substituted by halogen (such as fluorine, chlorine, bromine, or iodine) or C₁-C₆ alkyl.

In certain embodiments of the present application, [symbol] is a single bond or a double bond, and when [symbol] is a double bond, X and Y are each independently selected from CR^{B} or N; and when [symbol] is a single bond, X and Y are CR^{C}R^{D}, and R^{B}, R^{C}, and R^{D} are each independently selected from hydrogen, hydroxyl, halogen, cyano, C₃-C₈ cycloalkyl, C₁-C₆ alkyl, and C₁-C₆ haloalkyl. In some preferred embodiments, [symbol] is a single bond, X and Y are CR^{C}R^{D}, and the R^{C} and R^{D} are each independently selected from hydrogen, hydroxyl, and C₁-C₆ alkyl; preferably, the R^{C} and R^{D} are hydrogen. In some preferred embodiments, is a double bond, X and Y are each independently selected from CR^{B} and N, at least one of X and Y is CR^{B}, and the R^{B} is selected from hydrogen, C₃-C₈ cycloalkyl, C₁-C₆ alkyl, and C₁-C₆ haloalkyl; preferably, the R^{B} is selected from hydrogen, C₁-C₆ alkyl (preferably C₁-C₅ alkyl, C₁-C₄ alkyl, or C₁-C₃ alkyl), and C₁-C₆ haloalkyl (preferably C₁-C₅ haloalkyl, C₁-C₄ haloalkyl, or C₁-C₃ haloalkyl).

In certain embodiments of the present application, R^{A} is selected from hydrogen, hydroxyl, halo (such as fluoro, chloro, bromo, or iodo), an amine group, cyano, C₃-C₈ cycloalkyl (such as C₃-C₇ cycloalkyl, C₃-C₆ cycloalkyl, C₃-C₅ cycloalkyl, or C₃-C₄ cycloalkyl), C₁-C₆ alkyl (preferably C₁-C₅ alkyl, C₁-C₄ alkyl, or C₁-C₃ alkyl), C₁-C₆ alkoxy (such as C₁-C₅ alkoxy, C₁-C₄ alkoxy, or C₁-C₃ alkoxy), or C₁-C₆ haloalkyl (preferably C₁-C₅ haloalkyl, C₁-C₄ haloalkyl, or C₁-C₃ haloalkyl). In some preferred embodiments, R^{A} is selected from hydrogen, halo (such as fluoro, chloro, bromo, or iodo), an amine group, cyano, C₃-C₆ cycloalkyl (such as C₃-C₅ cycloalkyl, or C₃-C₄ cycloalkyl), C₁-C₆ alkyl (preferably C₁-C₅ alkyl, C₁-C₄ alkyl, or C₁-C₃ alkyl), C₁-C₆ alkoxy (such as C₁-C₅ alkoxy, C₁-C₄ alkoxy, or C₁-C₃ alkoxy), and C₁-C₆ haloalkyl (preferably C₁-C₅ haloalkyl, C₁-C₄ haloalkyl, or C₁-C₃ haloalkyl).

The present application further provides a compound represented by formula (II), a stereoisomer, tautomer or mixture form thereof, or a pharmaceutically acceptable salt thereof, or a solvate thereof, or a prodrug thereof:

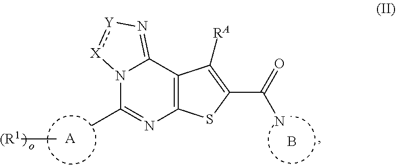

wherein R¹ and o are the same as defined above in the present application, and ring A is selected from an aromatic ring, an aromatic heterocycle, and an unsaturated aliphatic heterocycle;
ring B is a 3- to 12-membered saturated aliphatic heterocycle or a ring formed by a 3- to 12-membered saturated aliphatic heterocycle fused with a benzene ring;
[symbol] is a single bond or a double bond and when [symbol] is a double bond, X and Y are each independently selected from CR^{B} or N; when [symbol] is a single bond, X and Y are CR^{C}R^{D},
R^{A}, R^{B}, R^{C}, and R^{D} are each independently selected from hydrogen, hydroxyl, halogen, an amine group, cyano, C₃-C₈ cycloalkyl, C₁-C₆ alkyl, C₁-C₆ alkoxy, and C₁-C₆ haloalkyl;
the aromatic heterocycle, the saturated aliphatic heterocycle, the unsaturated aliphatic heterocycle, and the aliphatic heterocycle each independently include 1-3 heteroatoms, the heteroatom is independently selected from N, O, and S, and ring B includes at least 1 nitrogen atom;
the ring B is optionally substituted by one or two or more independently selected from deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6- to 10-membered aryl, and 5- to 10-membered heteroaryl.

In certain embodiments of the present application, o is 0 or 2, and when o is 2, any two R¹ and atoms of ring A connected thereto together form a 3- to 8-membered (such as 5- to 7-membered or 5- to 6-membered) aliphatic heterocyclic group. In some preferred embodiments, o is 0 or 2, when o is 2, any two R¹ and atoms of ring A connected thereto together form a 5- to 7-membered (preferably 5- to 6-membered) aliphatic heterocyclic group containing 1-3 heteroatoms (preferably 1-2 heteroatoms, or 1 heteroatom), and the heteroatom is independently N, O, or S (preferably N). In some preferred embodiments, the R¹ is optionally substituted by one or two independently selected from ═O, or C₁-C₆ alkyl (preferably C₁-C₅ alkyl, C₁-C₄ alkyl, or C₁-C₃ alkyl). In some preferred embodiments, o is 0 or 2, when o is 2, any two R¹ and atoms of ring A connected thereto together form saturated or unsaturated 5- to 6-membered cycloamido, and the cycloamido is unsubstituted or further substituted by C₁-C₆ alkyl (preferably C₁-C₅ alkyl, C₁-C₄ alkyl, or C₁-C₃ alkyl). In some preferred embodiments, o is 0 or 2, when o is 2, any two R¹ and atoms of ring A connected thereto together form saturated or unsaturated 5- to 6-membered cycloamido, and the cycloamido is optionally substituted by C₁-C₆ alkyl (preferably C₁-C₅ alkyl, C₁-C₄ alkyl, or C₁-C₃ alkyl) at an N atom therein.

Further, the ring B is preferably a monocycle, a fused ring, or a spirocycle. Preferably, the ring B is a 5- to 7-membered (preferably 5- to 6-membered) saturated aliphatic heterocycle containing 1-3 heteroatoms (preferably 1-2 heteroatoms), and the heteroatom is independently N, O, or S (preferably N). Further preferably, the ring B is a 5- to 6-membered saturated aliphatic heterocycle containing 1-2 heteroatoms (such as 1 heteroatom), the heteroatom is N, and the ring B is optionally substituted by halogen (such as fluorine, chlorine, bromine, or iodine) or C₁-C₆ alkyl (preferably, the ring B is optionally substituted by fluorine, chlorine, bromine, or iodine).

In certain embodiments of the present application, the above mentioned ring B is selected from

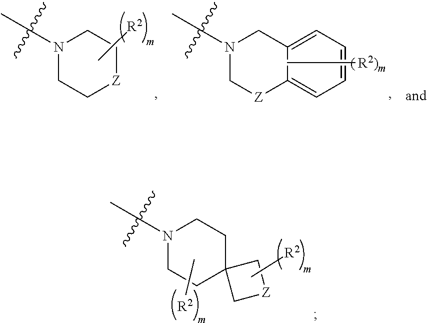

wherein Z is selected from a covalent bond, O, S, NH, (CH₂)ₙ, and SO₂;
m is selected from 0, 1, 2, and 3; R² is each independently selected from deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6- to 10-membered aryl, and 5- to 10-membered heteroaryl; the n is selected from 1, 2, and 3.

Further, in certain specific embodiments, the ring B is selected from

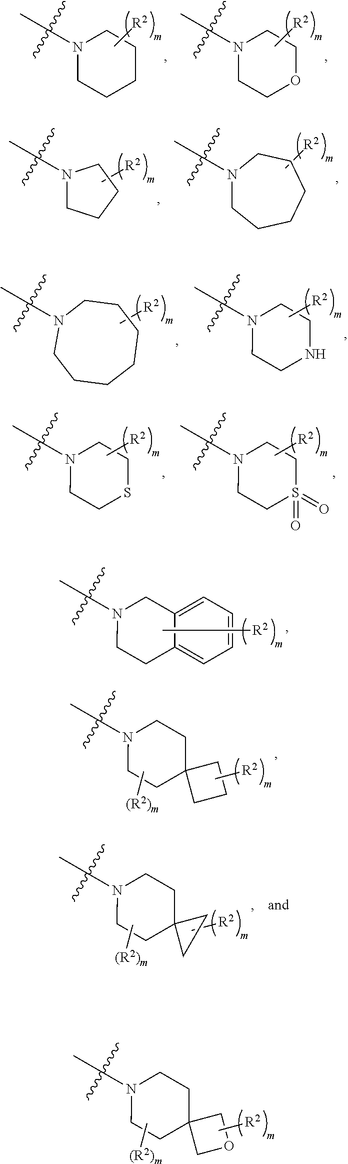

(such as

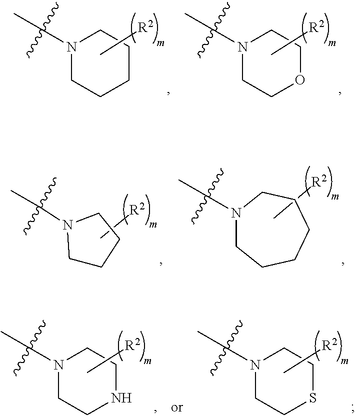

preferably

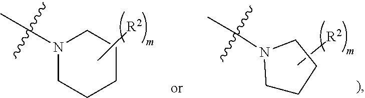

wherein m and R2 are the same as defined above in the present application.

Further, in certain specific embodiments of the present application, the R² is each independently selected from deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl. Preferably, the R² is each independently selected from fluoro, chloro, bromo, methyl, ethyl, n-propyl, and isopropyl, preferably fluoro, chloro, and bromo.

In certain embodiments of the present application, the [symbol] is a single bond, the X and Y are CR^{C}R^{D}, and the R^{C} and the R^{D} are selected from hydrogen, hydroxyl, cyano, halogen, methyl, ethyl, isopropyl, methoxy, ethoxy, trifluoromethyl, trifluoroethyl, trichloromethyl, trichloroethyl, cyclopropyl, cyclobutyl, and cyclopentyl. Alternatively preferably, the R^{C} and R^{D} are each independently selected from hydrogen, hydroxyl, or C₁-C₆ alkyl; for example, the R^{C} and R^{D} are hydrogen.

In certain embodiments of the present application, the [symbol] is a double bond, and at least one of the X and Y is selected from CR^{B}; in certain preferred embodiments of the present application, the [symbol] is a double bond, and the Y is selected from N, the X is selected from CR^{B}; in certain preferred embodiments of the present application, the [symbol] is a double bond, and the X is selected from N, the Y is selected from CR^{B}; in certain preferred embodiments of the present application, the [symbol] is a double bond, and both the X and Y are selected from CR^{B}.
wherein the R^{B} is selected from hydrogen, hydroxyl, cyano, halogen, C₃-C₈ cycloalkyl, C₁-C₆ alkyl, C₁-C₆ alkoxy, and C₁-C₆ haloalkyl; preferably, the R^{B} is selected from hydrogen, hydroxyl, cyano, halogen, methyl, ethyl, isopropyl, methoxy, ethoxy, trifluoromethyl, trifluoroethyl, trichloromethyl, trichloroethyl, cyclopropyl, cyclobutyl, and cyclopentyl. Alternatively preferably, the R^{B} is selected from hydrogen, C₃-C₈ cycloalkyl, C₁-C₆ alkyl, and C₁-C₆ haloalkyl; more preferably, the R^{B} is selected from hydrogen, C₁-C₆ alkyl (preferably C₁-C₅ alkyl, C₁-C₄ alkyl, or C₁-C₃ alkyl), and C₁-C₆ haloalkyl (preferably C₁-C₅ haloalkyl, C₁-C₄ haloalkyl, or C₁-C₃ haloalkyl).

In certain embodiments of the present application, R^{A} is selected from hydrogen, hydroxyl, halo (such as fluoro, chloro, bromo, or iodo), an amine group, cyano, C₃-C₈ cycloalkyl (such as C₃-C₇ cycloalkyl, C₃-C₆ cycloalkyl, C₃-C₅ cycloalkyl, or C₃-C₄ cycloalkyl), C₁-C₆ alkyl (preferably C₁-C₅ alkyl, C₁-C₄ alkyl, or C₁-C₃ alkyl), C₁-C₆ alkoxy (such as C₁-C₅ alkoxy, C₁-C₄ alkoxy, or C₁-C₃ alkoxy), or C₁-C₆ haloalkyl (preferably C₁-C₅ haloalkyl, C₁-C₄ haloalkyl, or C₁-C₃ haloalkyl). Preferably, R^{A} is selected from hydrogen, halo (such as fluoro, chloro, bromo, or iodo), an amine group, cyano, C₃-C₆ cycloalkyl (such as C₃-C₅ cycloalkyl, or C₃-C₄ cycloalkyl), C₁-C₆ alkyl (preferably C₁-C₅ alkyl, C₁-C₄ alkyl, or C₁-C₃ alkyl), C₁-C₆ alkoxy (such as C₁-C₅ alkoxy, C₁-C₄ alkoxy, or C₁-C₃ alkoxy), or C₁-C₆ haloalkyl (preferably C₁-C₅ haloalkyl, C₁-C₄ haloalkyl, or C₁-C₃ haloalkyl). Alternatively, in certain embodiments of the present application, the R^{A} is selected from hydrogen, hydroxyl, cyano, fluoro, chloro, bromo, iodo, —NH₂, methyl, ethyl, n-propyl, isopropyl, methoxy, ethoxy, trifluoromethyl, trifluoroethyl, trichloromethyl, trichloroethyl, cyclopropyl, cyclobutyl, or cyclopentyl.

In certain embodiments of the present application, the ring A is selected from a 6- to 10-membered aromatic ring, a 5- to 10-membered aromatic heterocycle, and a 6- to 8-membered unsaturated aliphatic heterocycle; preferably, the aromatic ring and the aromatic heterocycle are a monocycle or a fused ring, the unsaturated aliphatic heterocycle is a monocycle, the aromatic heterocycle and the unsaturated aliphatic heterocycle each independently include 1-3 heteroatoms, and the heteroatom is independently selected from N, O, and S. Preferably, the ring A is selected from a 6- to 10-membered aromatic ring, and a 5- to 10-membered aromatic heterocycle containing 1-3 heteroatoms (preferably 1-2 heteroatoms), and the heteroatom is independently selected from N, O, and S. More preferably, the ring A is a benzene ring, or a 5- to 10-membered aromatic heterocycle containing 1-2 heteroatoms, and the heteroatom is independently N or S.

In embodiments of the present application, the present application further provides a compound represented by formula (III), a stereoisomer, tautomer or mixture form thereof, or a pharmaceutically acceptable salt thereof, or a solvate thereof, or a prodrug thereof:

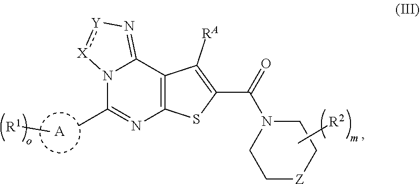

wherein R¹ and o are the same as defined above in the present application, ring A is selected from an aromatic ring, an aromatic heterocycle, and an unsaturated aliphatic heterocycle; Z is selected from a covalent bond, S, NH, CH₂, (CH₂)₂, and (CH₂)₃; m is selected from 0, 1, and 2; R² is each independently selected from deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6- to 10-membered aryl, and 5- to 10-membered heteroaryl; [symbol] is a single bond or a double bond, and when [symbol] is a double bond, X and Y are each independently selected from CR^{B} and N; when [symbol] is a single bond, X and Y are CR^{C}R^{D}; R^{A}, R^{B}, R^{C}, and R^{D} are each independently selected from hydrogen, hydroxyl, halogen, an amine group, cyano, C₃-C₈ cycloalkyl, C1-C₆ alkyl, C₁-C₆ alkoxy, and C₁-C₆ haloalkyl;
preferably, the R² is each independently selected from deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl. Preferably, the R² is each independently selected from fluoro, chloro, bromo, methyl, ethyl, n-propyl, and isopropyl, preferably fluoro, chloro, or bromo.

Further, in embodiments of the present application, the m may be 0 or 1.

further, in embodiments of the present application, the Z may be a covalent bond or CH₂; and in certain embodiments of the present application, the Z is CH₂.

In certain embodiments of the present application, the [symbol] is a single bond, the X and Y are CR^{C}R^{D}, and the R^{C} and R^{D} are selected from hydrogen, hydroxyl, cyano, halogen, methyl, ethyl, isopropyl, methoxy, ethoxy, trifluoromethyl, trifluoroethyl, trichloromethyl, trichloroethyl, cyclopropyl, cyclobutyl, and cyclopentyl. Alternatively preferably, the R^{C} and R^{D} are each independently selected from hydrogen, hydroxyl, and C₁-C₆ alkyl; and for example, the R^{C} and R^{D} are hydrogen.

In certain embodiments of the present application, the [symbol] is a double bond, and at least one of the X and Y is selected from CR^{B}; in certain preferred embodiments of the present application, the [symbol] is a double bond, and the Y is selected from N, while the X is selected from CR^{B}; in certain preferred embodiments of the present application, the [symbol] is a double bond, and the X is selected from N, while the Y is selected from CR^{B}; in certain preferred embodiments of the present application, the [symbol] is a double bond, and both the X and Y are selected from CR^{B};
wherein the R^{B} is selected from hydrogen, hydroxyl, cyano, halogen, C₃-C₈ cycloalkyl, C₁-C₆ alkyl, C₁-C₆ alkoxy, and C₁-C₆ haloalkyl; preferably, the R^{B} is selected from hydrogen, hydroxyl, cyano, fluoro, chloro, bromo, methyl, ethyl, isopropyl, methoxy, ethoxy, trifluoromethyl, trifluoroethyl, trichloromethyl, trichloroethyl, cyclopropyl, cyclobutyl, and cyclopentyl. Alternatively preferably, the R^{B} is selected from hydrogen, C₃-C₈ cycloalkyl, C₁-C₆ alkyl, and C₁-C₆ haloalkyl; more preferably, the R^{B} is selected from hydrogen, C₁-C₆ alkyl (preferably C₁-C₅ alkyl, C₁-C₄ alkyl, or C₁-C₃ alkyl), and C₁-C₆ haloalkyl (preferably C₁-C₅ haloalkyl, C₁-C₄ haloalkyl, or C₁-C₃ haloalkyl).

In certain embodiments of the present application, R^{A} is selected from hydrogen, hydroxyl, halo (such as fluoro, chloro, bromo, or iodo), an amine group, cyano, C₃-C₈ cycloalkyl (such as C₃-C₇ cycloalkyl, C₃-C₆ cycloalkyl, C₃-C₅ cycloalkyl, or C₃-C₄ cycloalkyl), C₁-C₆ alkyl (preferably C₁-C₅ alkyl, C₁-C₄ alkyl, or C₁-C₃ alkyl), C₁-C₆ alkoxy (such as C₁-C₅ alkoxy, C₁-C₄ alkoxy, or C₁-C₃ alkoxy), and C₁-C₆ haloalkyl (preferably C₁-C₅ haloalkyl, C₁-C₄ haloalkyl, or C₁-C₃ haloalkyl). Preferably, R^{A} is selected from hydrogen, halo (such as fluoro, chloro, bromo, or iodo), an amine group, cyano, C₃-C₆ cycloalkyl (such as C₃-C₅ cycloalkyl, or C₃-C₄ cycloalkyl), C₁-C₆ alkyl (preferably C₁-C₅ alkyl, C₁-C₄ alkyl, or C₁-C₃ alkyl), C₁-C₆ alkoxy (such as C₁-C₅ alkoxy, C₁-C₄ alkoxy, or C₁-C₃ alkoxy), and C₁-C₆ haloalkyl (preferably C₁-C₅ haloalkyl, C₁-C₄ haloalkyl, or C₁-C₃ haloalkyl). Alternatively, in embodiments of the present application, the R^{A} is selected from hydrogen, hydroxyl, cyano, fluoro, chloro, bromo, iodo, —NH₂, methyl, ethyl, n-propyl, isopropyl, methoxy, ethoxy, trifluoromethyl, trifluoroethyl, trichloromethyl, trichloroethyl, cyclopropyl, cyclobutyl, and cyclopentyl.

In embodiments of the present application, the ring A according to the present application is selected from a 6- to 10-membered aromatic ring, a 5- to 10-membered aromatic heterocycle, and a 6- to 8-membered unsaturated aliphatic heterocycle. Further, the aromatic ring and the aromatic heterocycle are preferably a monocycle or a fused ring, the unsaturated aliphatic heterocycle is preferably a monocycle, the aromatic heterocycle and the unsaturated aliphatic heterocycle each independently include 1-3 heteroatoms, and the heteroatom is independently selected from N, O, and S. Preferably, the ring A is selected from a 6- to 10-membered aromatic ring, and a 5- to 10-membered aromatic heterocycle containing 1-3 heteroatoms (preferably 1-2 heteroatoms), and the heteroatom is independently selected from N, O, and S. More preferably, the ring A is a benzene ring, or a 5- to 10-membered aromatic heterocycle containing 1-2 heteroatoms, and the heteroatom is independently N or S.

In certain embodiments of the present application, the ring A is selected from

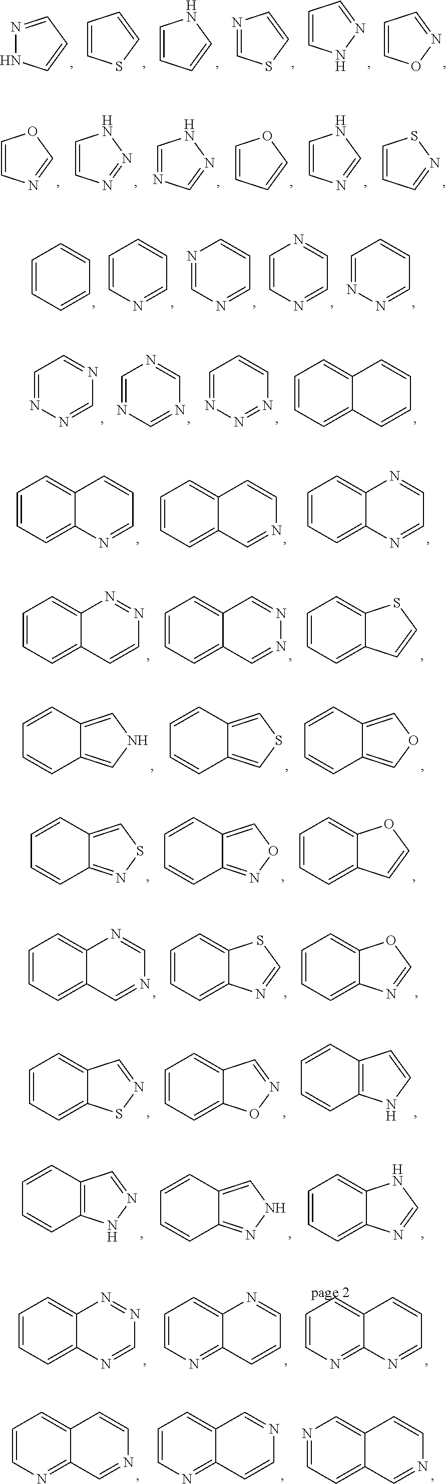

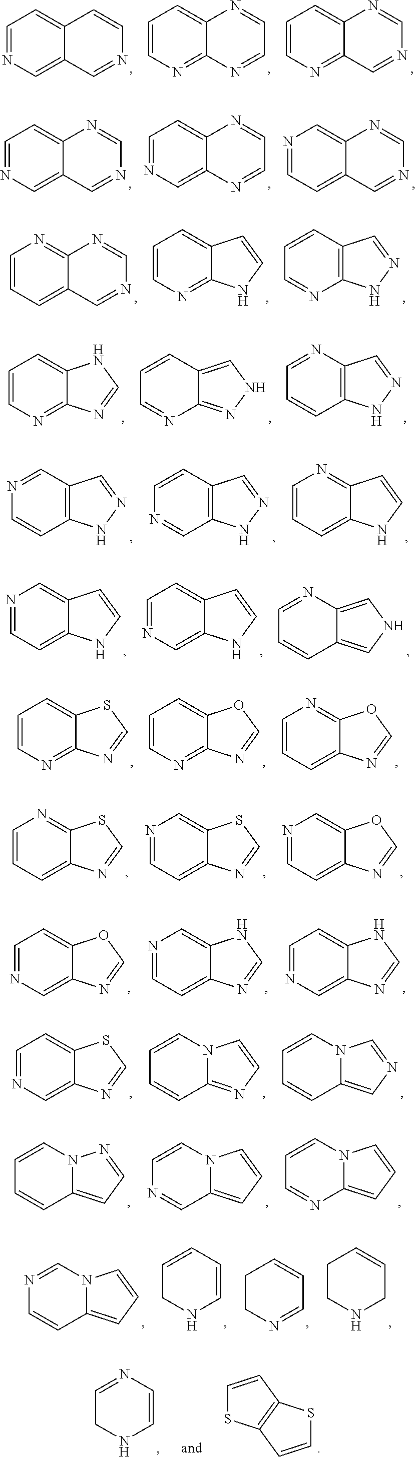

In certain embodiments of the present application, the ring A is selected from

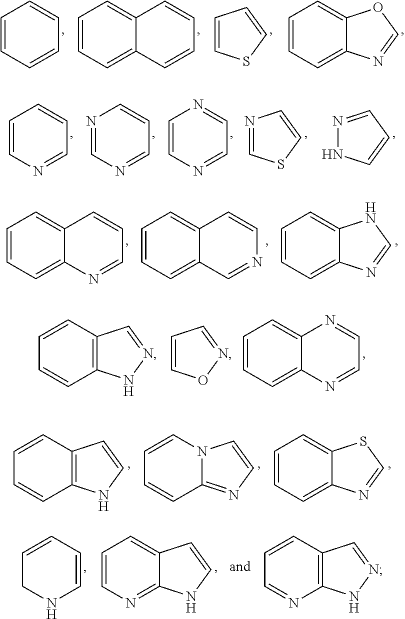

the ring A is more preferably selected from

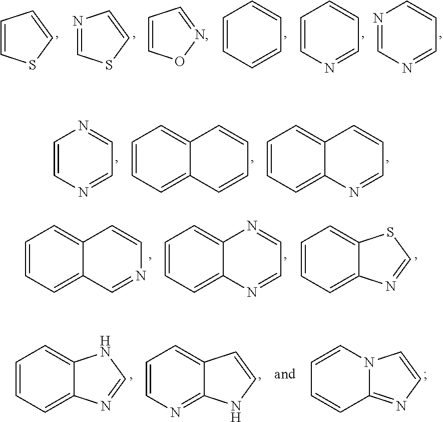

and the ring A is further selected from

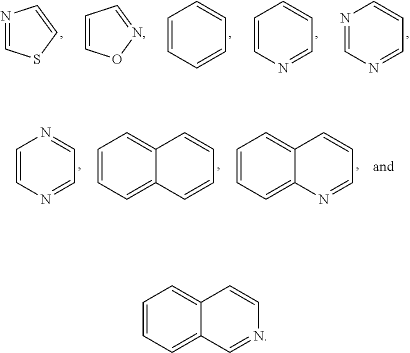

In certain embodiments of the present application, the R¹ according to the resent application is each independently selected from deuterium, tritium, nitro, hydroxyl, mercapto, halogen, cyano, ═O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, tert-butyl, n-pentyl, isopentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, tert-butoxy, n-pentyloxy, isopentyloxy, tert-pentyloxy, and n-hexyloxy; or any two R¹ and atoms of ring A connected thereto together form dioxanyl, dioxolanyl, dioxenyl, dioxolyl, dihydropyridyl, or pyrrolinyl, wherein the R¹ is optionally substituted by one or more independently selected from deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6- to 10-membered aryl, and 5- to 10-membered heteroaryl.

In certain embodiments of the present application, the R¹ according to the present application is each independently selected from deuterium, tritium, nitro, hydroxyl, mercapto, halogen, cyano, ═O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, tert-butyl, n-pentyl, isopentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, tert-butoxy, n-pentyloxy, isopentyloxy, tert-pentyloxy, and n-hexyloxy; or any two R¹ and atoms of ring A connected thereto together form dioxanyl or dioxolanyl, wherein the R¹ is optionally substituted by one or more independently selected from deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6- to 10-membered aryl, and 5- to 10-membered heteroaryl.

In certain specific embodiments of the present application, the R¹ according to the present application is each independently and preferably selected from deuterium, tritium, nitro, hydroxyl, mercapto, cyano, ═O, ═NH, —NH₂, —N(CH₃)₂, —NHCH₃, ═NCH₃, an ester group, an aldehyde group, carboxyl, amido, cyclopropyl, cyclobutyl, cyclohexyl, cyclopentyl, methyl, ethyl, isopropyl, trifluoromethyl, trifluoroethyl, trichloromethyl, trichloroethyl, morpholinyl, piperidinyl, N-methylpiperazinyl, p-methylpiperidinyl, piperazinyl, methoxy, ethoxy, isopropoxy, and halogen; or any two R¹ and atoms of ring A connected thereto together form 1,4-dioxanyl, 1,3-dioxanyl, 1,3-dioxolanyl, 1,4-dioxenyl, 1,3-dioxenyl, 1,3-dioxolyl, N-methyl-2-pyridinonyl, or N-methyl-3-pyrrolin-2-onyl; preferably, the R¹ according to the present application is each independently and preferably selected from deuterium, tritium, nitro, hydroxyl, mercapto, cyano, ═O, ═NH, —NH₂, —N(CH₃)₂, —NHCH₃, ═NCH₃, an ester group, an aldehyde group, carboxyl, amido, cyclopropyl, cyclobutyl, cyclohexyl, cyclopentyl, methyl, ethyl, isopropyl, trifluoromethyl, trifluoroethyl, trichloromethyl, trichloroethyl, morpholinyl, piperidinyl, N-methylpiperazinyl, p-methylpiperidinyl, piperazinyl, methoxy, ethoxy, isopropoxy, and halogen; or any two R¹ and atoms of ring A connected thereto together form 1,4-dioxanyl, 1,3-dioxanyl, or 1,3-dioxolanyl.

In certain embodiments of the present application, o is 0 or 2, and when o is 2, any two R¹ and atoms of ring A connected thereto together form a 3- to 8-membered (such as 5- to 7-membered or 5- to 6-membered) aliphatic heterocyclic group. In some preferred embodiments, o is 0 or 2, when o is 2, any two R¹ and atoms of ring A connected thereto together form a 5- to 7-membered (preferably 5- to 6-membered) aliphatic heterocyclic group containing 1-3 heteroatoms (preferably 1-2 heteroatoms, or 1 heteroatom), and the heteroatom is independently N, O, or S (preferably N). In some preferred embodiments, the R¹ is optionally substituted by one or two independently selected from ═O, and C₁-C₆ alkyl (preferably C₁-C₅ alkyl, C₁-C₄ alkyl, or C₁-C₃ alkyl). In some preferred embodiments, o is 0 or 2, when o is 2, any two R¹ and atoms of ring A connected thereto together form saturated or unsaturated 5- to 6-membered cycloamido, and the cycloamido is unsubstituted or further substituted by C₁-C₆ alkyl (preferably C₁-C₅ alkyl, C₁-C₄ alkyl, or C₁-C₃ alkyl). In some preferred embodiments, o is 0 or 2, when o is 2, any two R¹ and atoms of ring A connected thereto together form saturated or unsaturated 5- to 6-membered cycloamido, and the cycloamido is optionally substituted by C₁-C₆ alkyl (preferably C₁-C₅ alkyl, C₁-C₄ alkyl, or C₁-C₃ alkyl) at the N atom therein.

In some preferred embodiments, any two R¹ and atoms of ring A connected thereto together form N-methyl-2-pyridinonyl, N-methyl-5,6-dihydro-pyridin-2-onyl

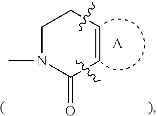

N-methyl-3,4-dihydro-pyridin-2-onyl

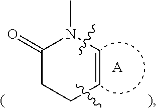

pyridin-2-onyl

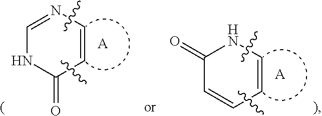

N-methyl-3-pyrrolin-2-onyl, pyrrol-2-onyl

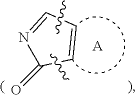

or 3-pyrrolin-2-onyl

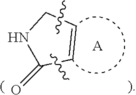

In certain preferred embodiments, the structures of groups formed by the two R¹ and atoms of ring A connected thereto are as follows: 1,4-dioxanyl having a structure of

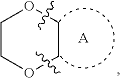

1,3-dioxolanyl having a structure of

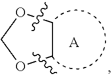

1,4-dioxenyl being able to have a structure of

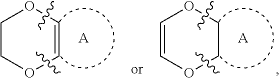

1 ,3-dioxenyl having a structure of

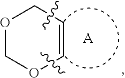

1,3-dioxolyl having a structure of

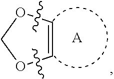

N-methyl-2-pyridinonyl being able to have a structure of

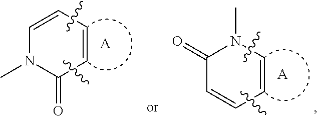

and N-methyl-3-pyrrolin-2-onyl having a structure of

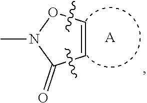

In some embodiments of the present application, in the compound represented by formula (III), the stereoisomer, tautomer or mixture form thereof, or the pharmaceutically acceptable salt thereof, or the solvate thereof (such as a hydrate), or the prodrug thereof, the ring A is a benzene ring, a naphthalene ring, or a 5- to 10-membered aromatic heterocycle containing 1-2 heteroatoms, and the heteroatom is independently N or S (for example, the ring A is selected from

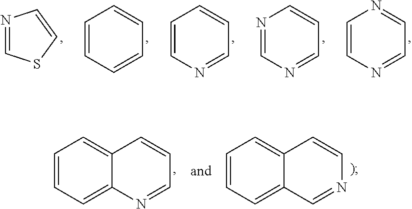

o is 0 or 2, when o is 2, any two R¹ and atoms of ring A connected thereto together form saturated or unsaturated 5- to 6-membered cycloamido, and the cycloamido is optionally substituted by C₁-C₆ alkyl (preferably C₁-C₅ alkyl, C₁-C₄ alkyl, or C₁-C₃ alkyl) at the N atom therein (for example, any two R¹ and atoms of ring A connected thereto together form N-methyl-2-pyridinonyl, N-methyl-5,6-dihydro-pyridin-2-onyl, N-methyl-3,4-dihydro-pyridin-2-onyl, pyridin-2-onyl, N-methyl-3-pyrrolin-2-onyl, pyrrol-2-onyl, or 3-pyrrolin-2-onyl); the [symbol] is a single bond, the X and Y are CR^{C}R^{D}, and the R^{C} and R^{D} are each independently selected from hydrogen, hydroxyl, and C₁-C₆ alkyl (for example, the R^{C} and R^{D} are hydrogen); or the [symbol] is a double bond, at least one of the X and Y is selected from CR^{B}, and the R^{B} is selected from hydrogen, C₃-C₈ cycloalkyl, C₁-C₆ alkyl, and C₁-C₆ haloalkyl (for example, the R^{B} is selected from hydrogen, C₁-C₆ alkyl (preferably C₁-C₅ alkyl, C₁-C₄ alkyl, or C₁-C₃ alkyl), and C₁-C₆ haloalkyl (preferably C₁-C₅ haloalkyl, C₁-C₄ haloalkyl, or C₁-C₃ haloalkyl)); R^{A} is selected from hydrogen, halo (such as fluoro, chloro, bromo, or iodo), an amine group, cyano, C₃-C₆ cycloalkyl (such as C₃-C₅ cycloalkyl, or C₃-C₄ cycloalkyl), C₁-C₆ alkyl (preferably C₁-C₅ alkyl, C₁-C₄ alkyl, or C₁-C₃ alkyl), C₁-C₆ alkoxy (such as C₁-C₅ alkoxy, C₁-C₄ alkoxy, or C₁-C₃ alkoxy), and C₁-C₆ haloalkyl (preferably C₁-C₅ haloalkyl, C₁-C₄ haloalkyl, or C₁-C₃ haloalkyl), for example, R^{A} is selected from hydrogen, fluoro, chloro, bromo, iodo, —NH₂, cyano, methyl, ethyl, n-propyl, isopropyl, methoxy, ethoxy, trifluoromethyl, trifluoroethyl, trichloromethyl, trichloroethyl, cyclopropyl, cyclobutyl, and cyclopentyl; the Z is a covalent bond or CH₂ (preferably, the Z is CH₂); the m is 0, 1, or 2; and the R² is each independently selected from fluoro, chloro, bromo, methyl, ethyl, n-propyl, and isopropyl (preferably, the R² is each independently fluoro, chloro, or bromo).

In some specific embodiments of the present application, the present application provides compounds shown below, stereoisomers, tautomers or mixture forms thereof, or pharmaceutically acceptable salts thereof, or solvates thereof, or prodrugs thereof:

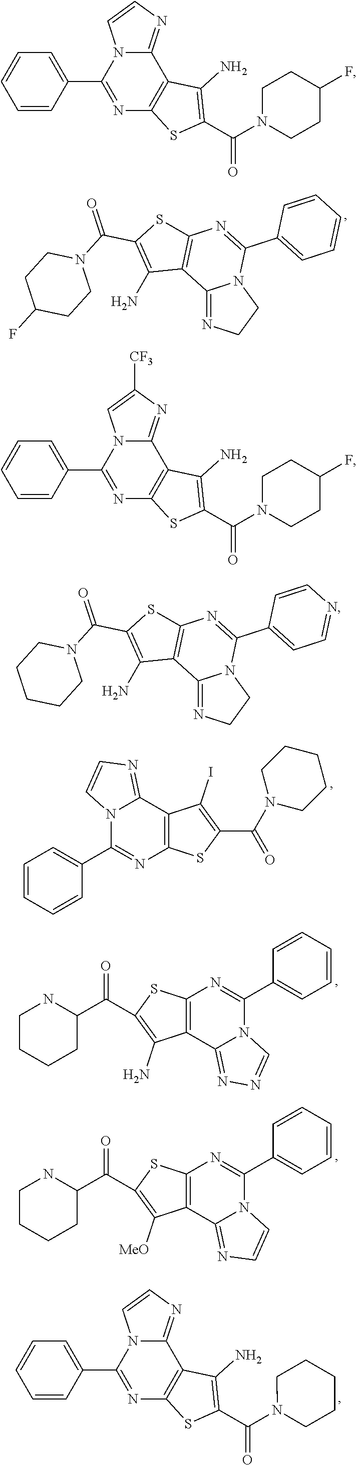

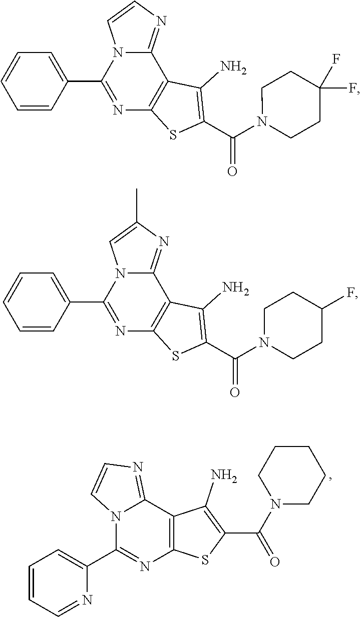 [SMILES: Nc1c(C(=O)N2CCC(F)(F)CC2)sc2nc(-c3ccccc3)n3ccnc3c12 ; Cc1cn2c(-c3ccccc3)nc3sc(C(=O)N4CCC(F)CC4)c(N)c3c2n1 ; Nc1c(C(=O)N2CCCCC2)sc2nc(-c3ccccn3)n3ccnc3c12]

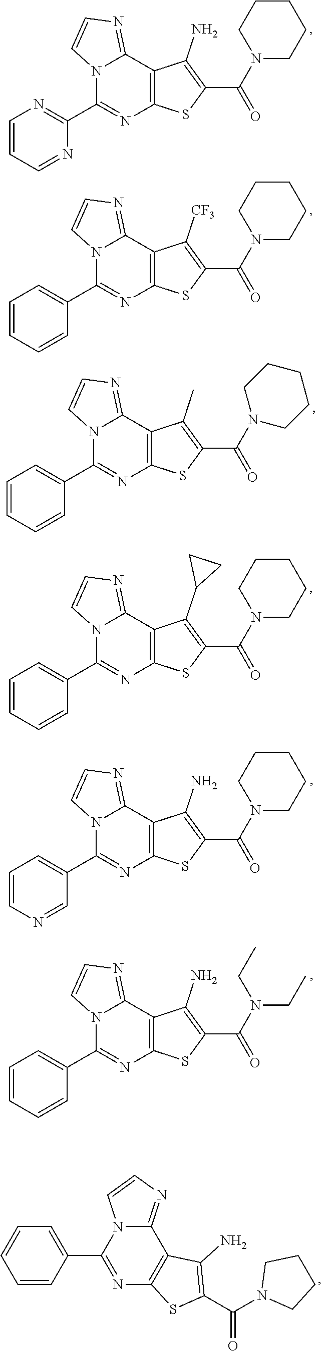

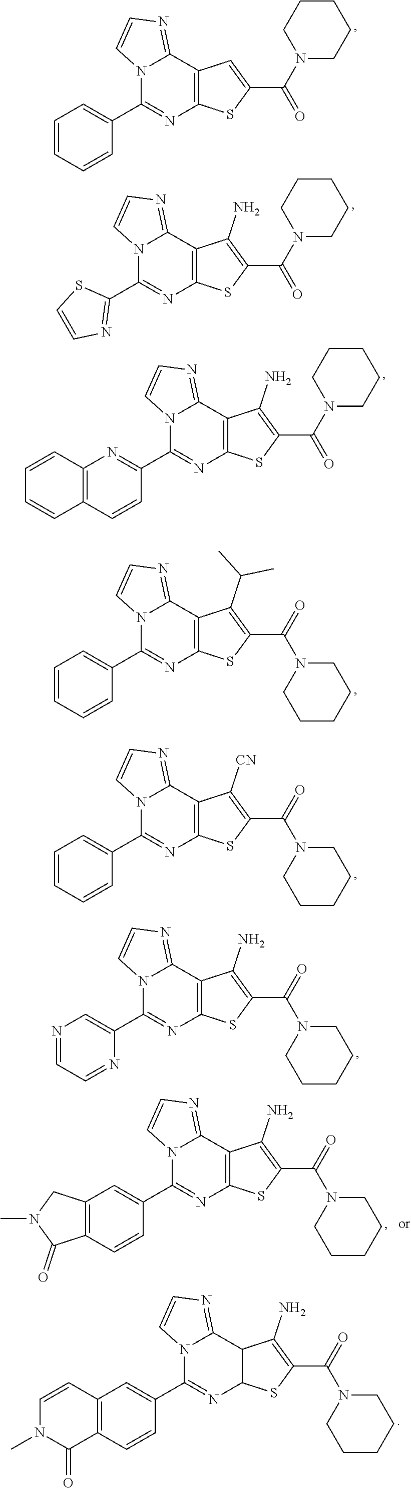

Another aspect of the present application provides a pharmaceutical composition, comprising at least one of the above-mentioned compounds, the stereoisomers, tautomers or mixture forms thereof, or the pharmaceutically acceptable salts thereof, or the solvates thereof, or the prodrugs thereof, and at least one pharmaceutically acceptable excipient.

Another aspect of the present application provides use of the above-mentioned compounds, or the stereoisomers, tautomers or mixture forms thereof, or the pharmaceutically acceptable salts thereof, or the solvates thereof, or the prodrugs thereof, or the pharmaceutical composition in the preparation of a medicament. Wherein the medicament is a 15-PGDH inhibitor which can be used for treating a disease associated with an undesirably increased activity level of 15-PGDH. Alternatively, the present application provides the above-mentioned compounds, or the stereoisomers, tautomers or mixture forms thereof, or the pharmaceutically acceptable salts thereof, or the solvates thereof, or the prodrugs thereof, or the pharmaceutical composition used as a medicament. Alternatively, the present application provides a method of treating or preventing a disease associated with 15-PGDH, comprising administering to a subject in need thereof the above-mentioned compounds, or the stereoisomers, tautomers or mixture forms thereof, or the pharmaceutically acceptable salts thereof, or the solvates thereof, or the prodrugs thereof, or the pharmaceutical composition. The disease associated with 15-PGDH herein refers to a disease or complication thereof for which a clinically beneficial effect, such as remission, amelioration, cessation of progression, alleviation, or no further deterioration, is achieved by inhibiting the activity of 15-PGDH.

In certain specific embodiments, the medicament or the method is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, and underactive bladder, or for promoting hair growth, pigmentation, tissue repair, tissue regeneration, implant in stem cell transplantation or bone marrow transplantation or organ transplantation, neurogenesis and neuronal cell death, muscle regeneration, and cervical ripening, or for enhancing resistance to the toxicity of chemotherapy and the toxicity of immunosuppressant.

### Definition

Unless otherwise stated, the following terms used in the specification and claims have the following meanings. A particular term or phrase shall not be considered uncertain or unclear in the absence of a specific definition, but should be understood according to its ordinary meaning.

“Chain hydrocarbon group” refers to an aliphatic group containing only carbon and hydrogen atoms connected in a chain. The hydrocarbon group may be a saturated hydrocarbon group or an unsaturated hydrocarbon group; and the chain may be a linear chain or a branched chain. The C₁-C₁₀ chain hydrocarbon group used in the present application refers to a linear hydrocarbon group or a branched hydrocarbon group consisted of 1-10 (such as 1, 2, 3, 4, 5, 6, 7, 8, 9, or 10, or a range value composed of any two of the preceding numerical values) carbon atoms, and the hydrocarbon group may be a saturated hydrocarbon group or an unsaturated hydrocarbon group.

“Alkyl” refers to a saturated aliphatic chain hydrocarbon group. The alkyl moiety may be a linear or branched alkyl; C₁-C₆ alkyl used herein refers to a linear or branched alkyl comprising 1 to 6 (e.g., 1, 2, 3, 4, 5, 6, or a range value composed of any two of the preceding numerical values) carbon atoms. Typical alkyl includes, but is not limited to, methyl, ethyl, n-propyl, isopropyl, n-butyl, isobutyl, t-butyl, n-pentyl, isopentyl, tert-amyl, n-hexyl, etc.

“Alkoxy” refers to —O-alkyl; C₁-C₆ alkoxy used herein refers to a linear or branched alkoxy group comprising 1 to 6 (e.g., 1, 2, 3, 4, 5, 6, or a range value composed of any two of the preceding numerical values) carbon atoms. Typical alkoxy includes, but is not limited to, methoxy, ethoxy, n-propoxy, isopropoxy, n-butoxy, isobutoxy, t-butoxy, n-pentyloxy, isopentyloxy, tert-pentyloxy, n-hexyloxy, etc.

“Ring” refers to any cyclic covalently closed structure, including, for example, a carbocycle (e.g., an aromatic or alicyclic ring), or a heterocycle (e.g., an aromatic or aliphatic heterocycle). The carbocycle refers to a ring only consisted of carbon atoms, and the heterocycle refers to a closed structure formed by covalently bonding carbon atoms and heteroatoms. The ring may be monocyclic, bicyclic, tricyclic or polycyclic. When the ring is a bicyclic, tricyclic or polycyclic ring, the relationship between individual rings may include a fused ring, a spirocycle, or a bridged cycle. For example, a bicyclic ring may include a spirocycle, a fused ring, and a bridged cycle, and a tricyclic ring may include a trispirocycle, a fused tricycle, a spirocycle fused monocycle, etc.

“Fused” in the present application refers to a structure formed by sharing two neighboring ring atoms between rings, for example, a fused ring refers to a cyclic structure formed by sharing two neighboring ring atoms between two monocycles.

“Heteroatom” refers to any atom, other than a carbon atom, that can be covalently bonded to a carbon atom. Common heteroatoms include, but are not limited to, O, S, N, P, Si, etc.

The “membered” refers to the number of skeleton atoms constituting a ring. A typical 5-membered ring may include, but is not limited to, cyclopentane, pyrrole, imidazole, thiazole, furan, thiophene, and the like, and a typical 6-membered ring includes, but is not limited to, cyclohexane, pyridine, pyran, pyrazine, thiapyran, pyridazine, pyrimidines, benzene, etc.

“Alicyclic ring” or “alicyclic group” refers to a saturated or partially unsaturated carbocycle; the saturated carbocycle may be referred to as, e.g., a saturated alicyclic ring; the partially unsaturated carbocycle may be referred to as, e.g., an unsaturated alicyclic ring; an alicyclic ring may be consisted of 3-10 atoms, and may be a monocycle or a polycycle; for example, the C₃-C₈ alicyclic group used in the present application refers to an alicyclic group consisted of 3-8 skeleton atoms. A typical alicyclic structure includes, but is not limited to:

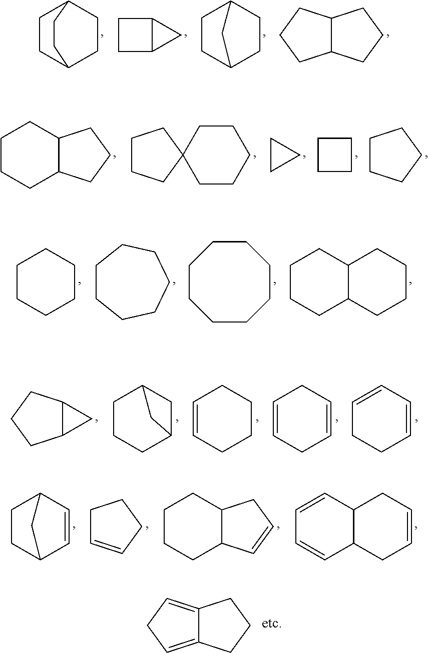

“Aliphatic heterocycle” or “aliphatic heterocyclic group” refers to a nonaromatic cyclic group formed by replacing carbon atom(s) in an alicyclic ring with one or more heteroatoms. The aliphatic heterocycle or aliphatic heterocyclic group may include a saturated aliphatic heterocycle and an unsaturated aliphatic heterocycle. For example, a 3- to 8-membered aliphatic heterocyclic group used in the present application refers to a nonaromatic cyclic group comprising one or more heteroatoms consisted of 3-8 skeleton atoms, and may be a saturated aliphatic heterocyclic group and an unsaturated aliphatic heterocyclic group.

“Saturated aliphatic heterocycle” or “saturated aliphatic heterocyclic group” means that the carbon atoms in the aliphatic heterocycle that form the ring skeleton are all saturated. For example, a 3- to 12-membered saturated aliphatic heterocycle used in the present application refers to a nonaromatic cyclic group formed by 3-12 atoms constituting the ring skeleton, wherein the atoms constituting the ring skeleton comprise saturated carbon atoms and heteroatoms. A typical saturated aliphatic heterocycle includes, but is not limited to:

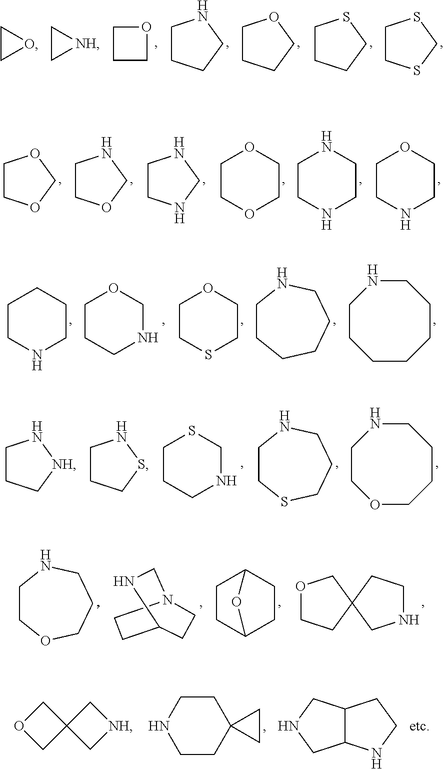

As used in the application, “a ring formed by a 3- to 12-member saturated aliphatic heterocycle fused with a benzene ring” refers to a cyclic structure consisted of a saturated aliphatic heterocycle of 3-12 atoms and a benzene ring in a fused means, for example,

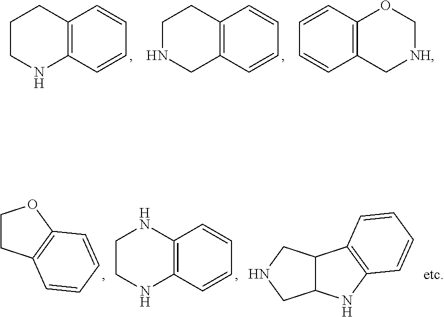

The “unsaturated aliphatic heterocycle” in the present application means that the skeleton of the aliphatic heterocycle contains unsaturated carbon atoms. A 6- to 8-membered unsaturated aliphatic heterocycle used in the present application refers to a nonaromatic cyclic group formed by 6-8 skeleton atoms, wherein the atoms constituting the ring skeleton include saturated carbon atoms, unsaturated carbon atoms, and heteroatoms, and a typical unsaturated aliphatic heterocycle includes, but is not limited to:

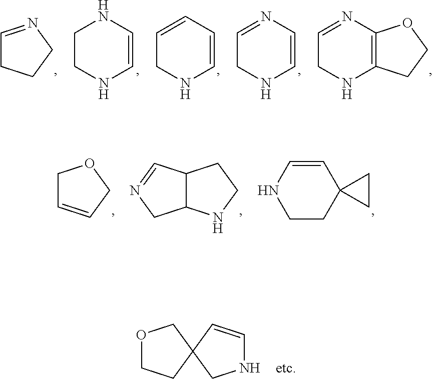

“Cycloalkyl” refers to a saturated aliphatic carbocyclic group, and may also be referred to as, for example, a saturated alicyclic ring. The cycloalkyl group may be a monocycle, a spirocycle, a fused ring or a bridged cycle. A C₃-C₈ cycloalkyl used in the application refers to a cyclic alkyl comprising 3-8 carbon atoms. A typical cycloalkyl includes, but is not limited to, cyclopropyl, cyclobutyl, cyclopentyl, cyclohexyl, bicyclo[2,1,1]hexyl, cycloheptyl, and the like.

“Aromatic ring” or “aryl” refers to a completely unsaturated carbocycle with a planar ring having a delocalized π-electron system and containing 4n+2 π electrons, where n is an integer. The aromatic ring may consist of six, eight, ten, or more than ten carbon atoms, and may be monocyclic or polycyclic. Common aromatic ring includes, but is not limited to, benzene ring, naphthalene ring, phenanthrene ring, anthracene ring, tetrabenzene, pyrene ring, pentabenzene, and the like. As used in the present application, a 6- to 10-membered aromatic ring or a 6- to 10-membered aryl group refers to an aromatic ring group consisting of 6-10 skeleton carbon atoms.

“Aromatic heterocycle” or “heteroaryl” refers to an aromatic cyclic structure formed by replacing carbon atoms in the aromatic ring with one or more heteroatoms, and a typical aromatic heterocycle or heteroaryl includes, but is not limited to:

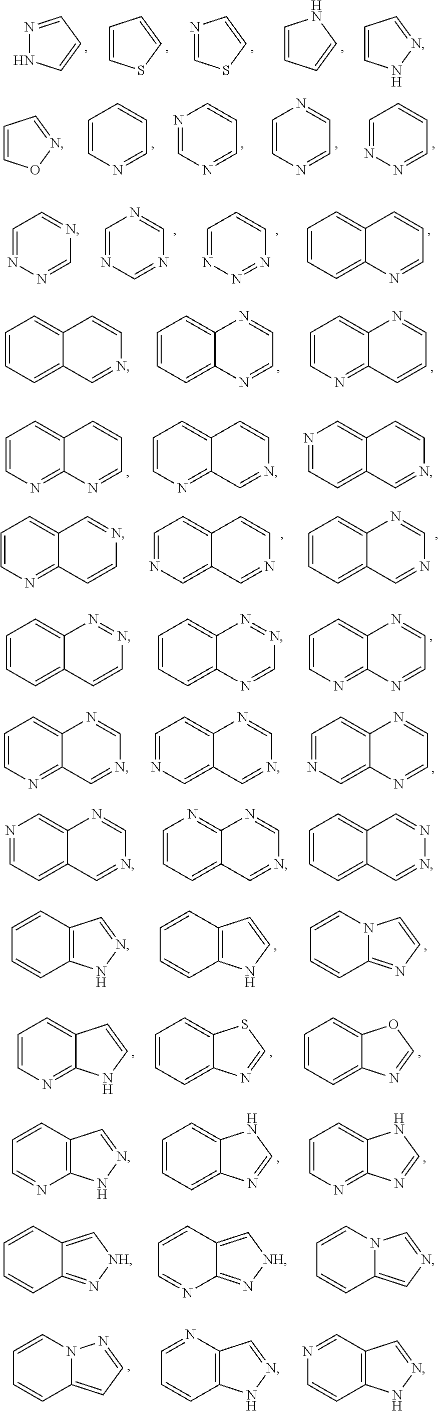

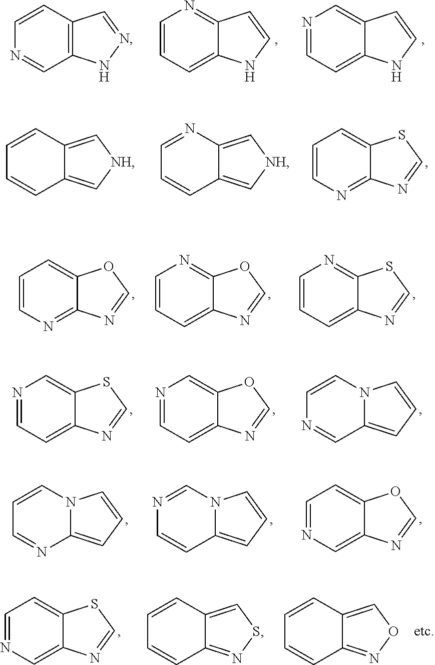

As used in the present application, a 5- to 10-membered aromatic heterocycle or a 5- to 10-membered heteroaryl refers to an aromatic ring group comprising heteroatom(s) consisted of 5-10 skeleton carbon atoms.

The “halogen” or “halo” refers to fluorine, chlorine, bromine or iodine.

“Haloalkyl” means that at least one hydrogen in an alkyl group is replaced by a halogen atom, and a C₁-C₆ haloalkyl, as used in this application, means a linear or branched alkyl consisting of 1-6 carbon atoms and at least one hydrogen in the alkyl is arbitrarily replaced by a halogen atom.

“Amine group” or “amine” means having a chemical structure of —NR^{U}R^{V}, wherein R^{U}, R^{V} are each independently selected from hydrogen, deuterium, tritium, alkyl, cycloalkyl.

“Imino” or “imine” means having a chemical structure of ═NR^{W}, wherein R^{W} is selected from hydrogen, deuterium, tritium, alkyl, cycloalkyl.

“Amide” or “amido” means having a chemical structure of —C(O)NR^{X}R^{Y} or —NR^{X}C(O)R^{Y}, wherein R^{X}, R^{Y} are each independently selected from hydrogen, deuterium, tritium, alkyl, cycloalkyl, and common amido includes, but is not limited to —CONH₂, —CONHCH₃, —CON(CH₃)₂, —NHCOH, —NHCOCH₃, —N(CH₃)COCH₃.

“Ester group” means having a chemical structure of a formula of —COOR⁰, wherein R⁰ is selected from alkyl, cycloalkyl, heterocycloalkyl, aryl, heteroaryl.

“Substituted” means that one or more hydrogen atoms in a group are substituted independently by a corresponding number of substituents. It goes without saying that, the substituents are only in their possible chemical positions, and those of skills in the art are able to determine (either experimentally or theoretically) possible or impossible substitutions without undue effort. For example, an amino or hydroxyl with a free hydrogen may be unstable when binds to a carbon atom with an unsaturated (e.g. olefinic) bond. Each is independently selected from alkyl, cycloalkyl, aryl, heteroaryl, heterocycloalkyl, hydroxy, alkoxy, alkylthio, aryloxy, nitro, acyl, halogen, haloalkyl, amino, and the like. When two or more “substitutions” occur, the substituents may form a cyclic group together with the substituted atoms. For example, in the present application, two R¹ and atoms of ring A connected thereto together form 1,4-dioxanyl having a structure of

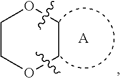

1,3-dioxolanyl having a structure of

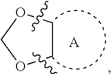

1,4-dioxenyl being able to have a structure of

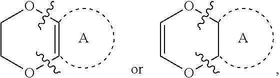

1,3-dioxenyl having a structure of

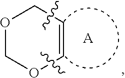

1,3-dioxolyl having a structure of

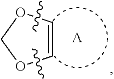

N-methyl-2-pyridinonyl being able to have a structure of

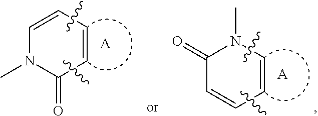

and N-methyl-3-pyrrolin-2-onyl having a structure of

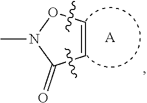

“Inhibitor” refers to a substance reducing the activity of an enzyme.

“Optional” or “optionally” means that the event or circumstance subsequently described may, but not necessarily, occur, and the description includes a situation when the event or circumstance does or does not occur. For example, “optionally substituted” includes substituted or unsubstituted, e.g., “a heterocyclic group optionally substituted by an alkyl” means that the alkyl may, but not necessarily, be present, and the description includes a situation in which the heterocyclic group is substituted by the alkyl and a situation in which the heterocyclic group is not substituted by the alkyl.

“Pharmaceutical composition” indicates a mixture comprising one or more of the compounds described herein, or a physiologically/pharmaceutically acceptable salt or prodrug thereof, with other chemical components, as well as other components such as physiologically/pharmaceutically acceptable carriers and vehicles. The pharmaceutical composition is intended to facilitate administration to an organism and facilitate absorption of an active ingredient to exert the biological activity.

“Pharmaceutically acceptable” refers to those compounds, materials, compositions and/or dosage forms that are suitable for use in contact with human and animal tissues within a range of sound medical judgment, without undue toxicity, irritation, allergic response or other problems or complications, commensurate with a reasonable benefit/risk ratio.

As pharmaceutically acceptable salts, mention may be made of, e.g., metal salts, ammonium salts, salts formed with organic bases, salts formed with inorganic acids, salts formed with organic acids, salts formed with basic or acidic amino acids, and the like.

“Tautomer” or “tautomeric form” refers to structural isomers of different energies that can be interconverted through low-energy barriers. For example, proton tautomer (also known as proton transfer tautomer) includes tautomerism via proton migration, such as keto-enol and imine-enamine isomerization. Specific example of proton tautomer is imidazole moiety, wherein the proton can migrate between the two ring nitrogens. Valence tautomers include interconversion by recombination of some bonding electrons. Non-limiting examples of tautomers include, but are not limited to

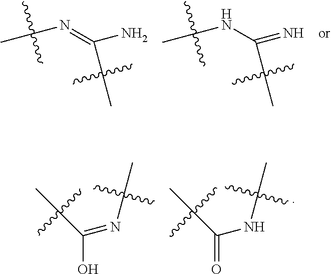 [SMILES: */N=C(\*)N ; *NC(*)=N ; */N=C(\*)O ; *NC(*)=O]

“Stereoisomer” refers to an isomer resulting from a different spatial arrangement of atoms in a molecule.

“Enantiomer” refers to isomerism caused by different spatial configurations of the atoms of compounds having the same molecular formula and functional groups, and said compounds form stereoisomers that are mirror images of each other and cannot overlap.

“Diastereoisomer” refers to isomerism caused by different spatial configurations of the atoms of compounds having the same molecular formula and functional groups, and said compounds are stereoisomers that do not exhibit a physical or mirror image relationship with each other.

Unless otherwise indicated, the terms “comprise, comprises and comprising” or their equivalents (contain, contains, containing, include, includes, including) used herein are open-ended mode expressions, and mean that other unspecified elements, components and steps may also be covered, in addition to the elements, components and steps listed.

Unless otherwise indicated, all numbers used herein to denote amounts of ingredients, measurements, or reaction conditions should be understood to be modified in all cases by the term “about”. When associated with a percentage, the term “about” may indicate, for example, ±1%, preferably ±0.5%, more preferably ±0.1%.

Unless otherwise specified clearly in the context, singular terms herein cover plural referents, and vice versa. Similarly, unless otherwise specified clearly in the context, the word “or” herein is intended to include “and”.

Apparently, according to the above contents of the application, in accordance with the ordinary technical knowledge and means in the field, under the premise of not departing from the above basic technical concepts of the application, a variety of other forms of modifications, substitutions or changes can also be made.

The abbreviations in the application have the meanings indicated below:

| | |
|---|---|
| K₂CO₃ represents potassium carbonate; | THF represents tetrahydrofuran; |
| POCl₃ represents phosphorus oxychloride; | NaH represents sodium hydride; |
| CS₂ represents carbon disulfide; | DMA or DMAc represents N,N-dimethylacetamide; |
| MeI represents methyl iodide; | ACN represents acetonitrile; |
| HATU represents 2-(7-azabenzotriazol-1-yl)- | Pd(dppf)Cl₂ represents [1,1′- |
| N,N,N′,N′-tetramethyluronium hexafluorophosphate; | bis(diphenylphosphino)ferrocene]dichloropalladium; |
| TEA represents triethylamine; | TsCl represents p-toluenesulfony1 chloride; |
| DMAP represents 4-dimethylaminopyridine; | DDQ represents 2,3-dichloro-5,6- |
| | dicyanobenzoquinone; |
| NH₃ represents ammonia or a solution of ammonia; | DMF represents N,N-dimethylformamide; |
| NaOH represents sodium hydroxide; | EtOH/H₂O represents an ethanol-water solution; |
| NH₂NH₂ represents hydrazine hydrate; | MW represents a microwave reaction; |
| rt represents a room temperature reaction; | mCPBA represents m-chloroperoxybenzoic acid; |
| DCM represents dichloromethane; | Pd-C represents palladium on carbon; |
| LiOH represents lithium hydroxide; | Cs₂CO₃ represents cesium carbonate; |
| NaHS represents sodium hydrosulfide; | LiHMDS represents lithium hexamethyldisilazide; |
| v/v represents a volume ratio. | |

### EMBODIMENTS OF THE INVENTION

The methods of synthesizing the compounds and intermediates of the present application are described below by way of example. The following examples are only intended to serve as examples of the present application, and should not be taken as a limitation to the scope of the present application. Unless otherwise indicated, the raw materials and reagents involved in the present application are all available commercially, and the specific source does not affect the implementation of the technical solution of the present application.

### Preparation Example 1: Preparation of Sodium 2-oxo-2-(piperidin-1-yl)ethane-1-thiolate

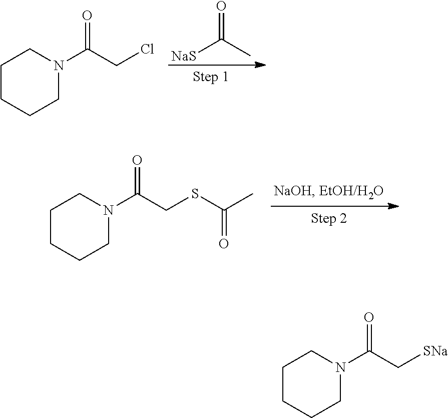

### Step 1: Preparation of S-(2-oxo-2-(piperidin-1-yl)ethyl)thioacetate

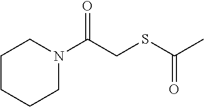

2-chloro-1-piperidin-1-ylethanone (10.0 g) was dissolved in acetonitrile (100 mL), into which sodium thioacetate (12.1 g) was then added, and stirred overnight at room temperature. When a reaction monitored by LCMS was completed, water was added to quench the reaction, concentration was performed under reduced pressure to remove acetonitrile, and extracted with ethyl acetate (100 mL) for three times. Organic phases were combined, dried over anhydrous sodium sulfate, filtered, and concentrated under reduced pressure, and a residue was purified by silica gel column chromatography to obtain 10.5 g of the title compound. MS (ESI) m/z (M+H)⁺=202.1.

### Step 2: Preparation of sodium 2-oxo-2-(piperidin-1-yl)ethane-1-thiolate

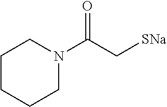

The S-(2-oxo-2-(piperidin-1-yl)ethyl)thioacetate (10.5 g) was dissolved in a mixed solution of ethanol and water (EtOH:H₂O=2:1 (v/v)), into which sodium hydroxide (6.3 g) was then added, and stirred at room temperature for 2 hours. When a reaction monitored by LCMS was completed, the reaction was stopped to obtain an ethanol-water solution of the target compound with a concentration of about 1 mmoL/mL, which was ready for use without further purification. MS (ESI) m/z (M+H)⁺=160.1.

### Preparation Example 2: Preparation of ethyl 5-iodo-4-(methylthio)-2-phenylthieno[2,3-d]pyrimidine-6-carboxylate

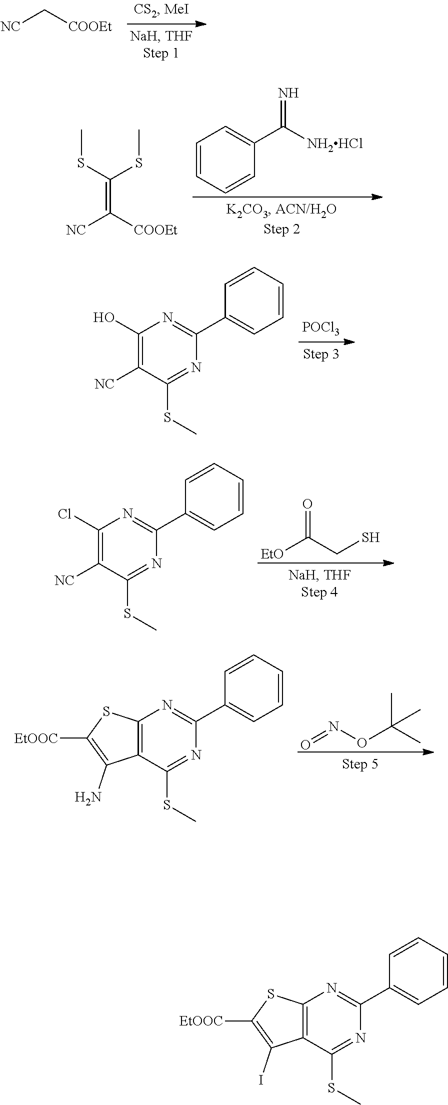

### Step 1: Preparation of ethyl 2-cyano-3,3-bis(methylthio)acrylate

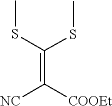

NaH (5.3 g) was dissolved in tetrahydrofuran (100 mL), into which ethyl cyanoacetate (10.0 g) was added under the protection of nitrogen, and then carbon disulfide (5.3 mL) was slowly added thereto dropwise under a dry ice bath for cooling to −78° C., and after the dropping was completed, stirring was performed continuously at −78° C. to react for 1 hour. When the system became light yellow, iodomethane (13.7 mL) was slowly added dropwise, after the dropping was completed, stirring was performed continuously at −78° C. to react for 1 hour, and then it was slowly raised to 0° C. to react for 1 hour. When the reaction monitored by LCMS was completed, water was added to quench the reaction, and concentration was performed under reduced pressure to remove tetrahydrofuran. A residue was poured into ice water and stirred continuously to precipitate a yellow solid, which was subjected to suction filtration and washed with water, and a product was dried to obtain 13 g of the title compound. MS (ESI) m/z (M+H)⁺=218.1.

### Step 2: Preparation of 4-hydroxy-6-(methylthio)-2-phenylpyrimidine-5-carbonitrile

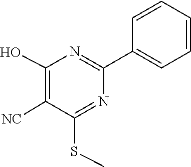 [SMILES: CSc1nc(-c2ccccc2)nc(O)c1C#N]

The ethyl 2-cyano-3,3-bis(methylthio)acrylate (13 g), benzamidine hydrochloride (7.9 g), and potassium carbonate (24.8 g) were dissolved in a mixed solution of acetonitrile and water (ACN:H₂O=4:1 (v/v)) and stirred at room temperature to react for 1 hour. When the reaction monitored by LCMS was completed, concentration was performed under reduced pressure to remove the organic solvents and precipitate a solid, which was filtrated, and a filter cake was washed with water and subjected to vacuum drying to obtain 16 g of the title compound. MS (ESI) m/z (M+H)⁺=244.1.

### Step 3: Preparation of 4-chloro-6-(methylthio)-2-phenylpyrimidine-5-carbonitrile

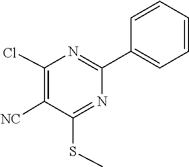 [SMILES: CSc1nc(-c2ccccc2)nc(Cl)c1C#N]

The crude 4-hydroxy-6-(methylthio)-2-phenylpyrimidine-5-carbonitrile (16 g) was dissolved in phosphorus oxychloride (100 mL), heated to 100° C., and stirred to react for 2 hours. When the reaction monitored by LCMS was completed, concentration was performed under reduced pressure to remove phosphorus oxychloride. A residue was poured into ice water, adjusted to weakly alkaline with a saturated NaHCO₃ solution, and extracted with ethyl acetate (100 mL) for three times. Organic phases were combined, dried over anhydrous sodium sulfate, filtered, and concentrated under reduced pressure, and a residue was purified by silica gel column chromatography to obtain 8 g of the title compound. MS (ESI) m/z (M+H)⁺=262.1.

### Step 4: Preparation of ethyl 5-amino-4-(methylthio)-2-phenylthieno[2,3-d]pyrimidine-6-carboxylate

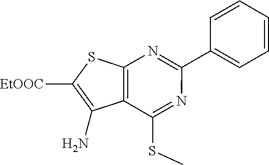 [SMILES: CCOC(=O)c1sc2nc(-c3ccccc3)nc(SC)c2c1N]

Sodium hydride (2.4 g) was dissolved in tetrahydrofuran (50 mL), into which ethyl thioglycolate (1.7 mL) was slowly added dropwise in an ice salt bath under the protection of nitrogen, and after the dropping was completed, a reaction was carried out for 0.5 hours. Then, the 4-chloro-6-(methylthio)-2-phenylpyrimidine-5-carbonitrile (2 g) was slowly added and continuously stirred under the ice salt bath to react for 1 hour. When the reaction monitored by LCMS was completed, a saturated ammonium chloride solution was added to quench the reaction, concentration was performed under reduced pressure to remove tetrahydrofuran, and a residue was extracted with ethyl acetate (50 mL) for three times. Organic phases were combined, dried over anhydrous sodium sulfate, filtered, and concentrated under reduced pressure, and a residue was purified by silica gel column chromatography to obtain 2.3 g of the title compound. MS (ESI) m/z (M+H)⁺=346.1.

### Step 5: Preparation of ethyl 5-iodo-4-(methylthio)-2-phenylthieno[2,3-d]pyrimidine-6-carboxylate

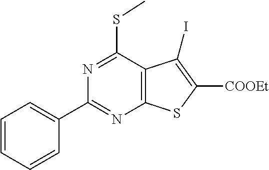 [SMILES: CCOC(=O)c1sc2nc(-c3ccccc3)nc(SC)c2c1I]

The ethyl 5-amino-4-(methylthio)-2-phenylthieno[2,3-d]pyrimidine-6-carboxylate (2.0 g) was added into acetonitrile (50 mL), into which cuprous iodide (2.2 g) was added, it was raised to 60° C., tert-butyl nitrite (1.2 g) was added thereto dropwise, and after the dropping was completed, stirring was performed continuously at 60° C. to react for 2 hours. When raw materials monitored by LCMS depleted, the reaction was stopped. The system was concentrated under reduced pressure, and a residue was purified by silica gel column chromatography to obtain 1.0 g of the title compound. MS (ESI) m/z (M+H)⁺=457.1.

### Preparation Example 3: Preparation of ethyl 4-(methylthio)-2-phenyl-5-(trifluoromethyl)thieno[2,3-d]pyrimidine-6-carboxylate

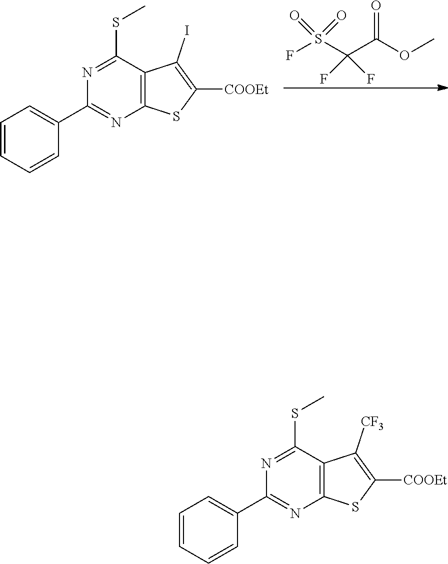 [SMILES: CCOC(=O)c1sc2nc(-c3ccccc3)nc(SC)c2c1I ; COC(=O)C(F)(F)S(=O)(=O)F ; CCOC(=O)c1sc2nc(-c3ccccc3)nc(SC)c2c1C(F)(F)F]

Ethyl 5-iodo-4-(methylthio)-2-phenylthieno[2,3-d]pyrimidine-6-carboxylate (0.3 g) was added into N,N-dimethylformamide (5 mL), into which cuprous iodide (30 mg) and methyl fluorosulfonyldifluoroacetate (0.4 g) were added, after nitrogen replacement for three times, it was raised to 90° C., and stirred to react for 2 hours. The reaction monitored by LCMS was completed. The system was concentrated under reduced pressure, and a residue was purified by silica gel column chromatography to obtain 120 mg of the title compound. MS (ESI) m/z (M+H)⁺=399.1.

### Preparation Example 4: Preparation of ethyl 5-cyclopropyl-4-(methylthio)-2-phenylthieno[2,3-d]pyrimidine-6-carboxylate

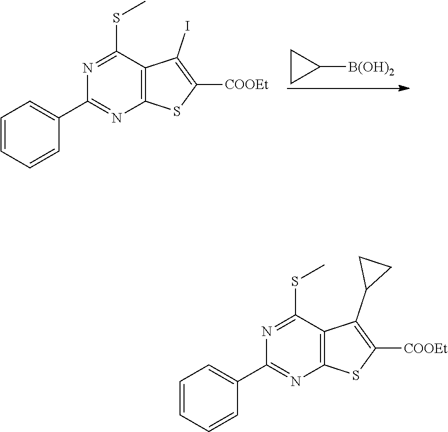 [SMILES: CCOC(=O)c1sc2nc(-c3ccccc3)nc(SC)c2c1I ; OB(O)C1CC1 ; CCOC(=O)c1sc2nc(-c3ccccc3)nc(SC)c2c1C1CC1]

Ethyl 5-iodo-4-(methylthio)-2-phenylthieno[2,3-d]pyrimidine-6-carboxylate (300 mg) was dissolved in a mixed solution of dioxane and water (dioxane:H₂O=4:1 (v/v)), and then potassium carbonate (182 mg), [1,1′-bis(diphenylphosphino)ferrocene]dichloropalladium (48 mg), and cyclopropyl boronic acid (100 mg) were added thereto, after nitrogen replacement for three times, it was raised to 90° C., and stirred to react for 2 hours. When the reaction monitored by LCMS was completed, the system was concentrated under reduced pressure, and a residue was purified by silica gel column chromatography to obtain 160 mg of the title compound. MS (ESI) m/z (M+H)⁺=371.1.

### Preparation Example 5: Preparation of ethyl 4-(methylthio)-2-phenylthieno[2,3-d]pyrimidine-6-carboxylate

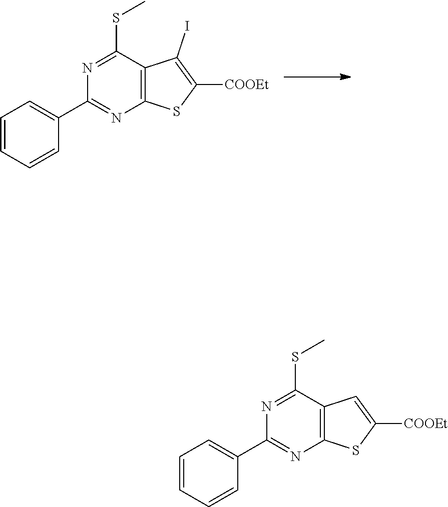 [SMILES: CCOC(=O)c1sc2nc(-c3ccccc3)nc(SC)c2c1I ; CCOC(=O)c1cc2c(SC)nc(-c3ccccc3)nc2s1]

Ethyl 5-iodo-4-(methylthio)-2-phenylthieno[2,3-d]pyrimidine-6-carboxylate (0.3 g) was added into N,N-dimethylformamide (5 mL), into which cuprous iodide (30 mg) was added, after nitrogen replacement for three times, it was raised to 90° C., and stirred to react for 2 hours. The reaction monitored by LCMS was completed. The system was concentrated under reduced pressure, and a residue was purified by silica gel column chromatography to obtain 100 mg of the title compound. MS (ESI) m/z (M+H)⁺=331.1.

### Preparation Example 6: Preparation of ethyl 5-methyl-4-(methylthio)-2-phenylthieno[2,3-d]pyrimidine-6-carboxylate

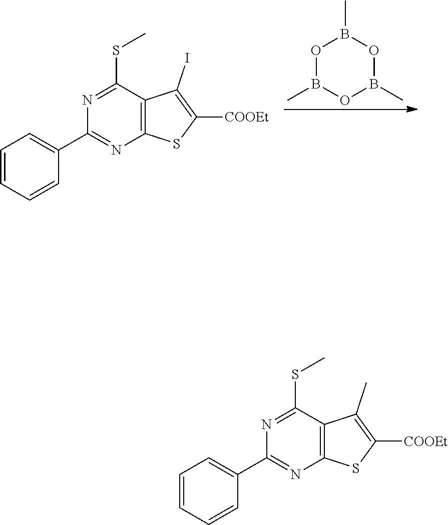

Ethyl 5-iodo-4-(methylthio)-2-phenylthieno[2,3-d]pyrimidine-6-carboxylate (0.3 g) was dissolved in dioxane (8 mL), and then cesium carbonate (40 mg), a [1,1′-bis(diphenylphosphino)ferrocene]dichloropalladium dichloromethane complex (5 mg), and trimethylboroxine (15 mg) were added thereto, it was raised to 100° C. under the protection of nitrogen, and stirred to react for 3 hours. When the reaction monitored by LCMS was completed, the system was concentrated under reduced pressure, and a residue was purified by silica gel column chromatography to obtain 100 mg of the title compound. MS (ESI) m/z (M+H)⁺=345.1.

### Example 1: Preparation of (9-amino-5-phenylimidazo[1,2-c]thieno[3,2-e]pyrimidin-8-yl)(4-fluoropiperidin-1-yl)methanone

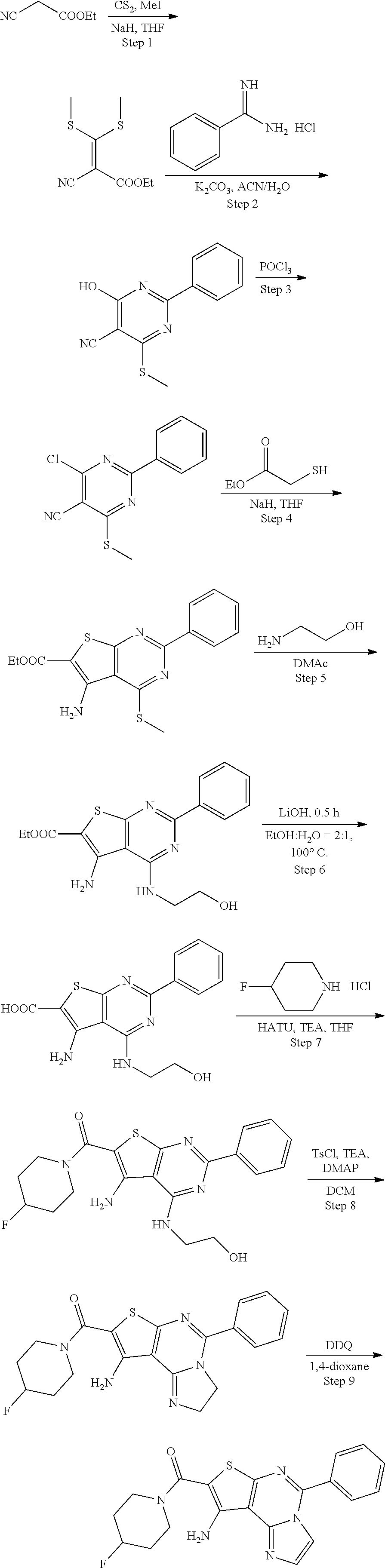

### Step 1: Preparation of ethyl 2-cyano-3,3-bis(methylthio)acrylate

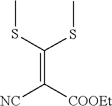

Sodium hydride (5.3 g) was dissolved in tetrahydrofuran (100 mL), into which ethyl cyanoacetate (10.0 g, 88.41 mmol) was added under the protection of nitrogen, then carbon disulfide (5.3 mL) was slowly added dropwise under a dry ice bath for cooling to −78° C., and after the dropping was completed, stirring was performed continuously at −78° C. to react for 1 hour. When the system became light yellow, iodomethane (13.7 mL) was slowly added dropwise, after the dropping was completed, stirring was still performed continuously at −78° C. to react for 1 hour, and then it was slowly raised to 0° C. to react for 1 hour. When the reaction monitored by LCMS was completed, water was added to quench the reaction, and concentration was performed under reduced pressure to remove tetrahydrofuran. A residue was poured into ice water and stirred continuously to precipitate a yellow solid, which was subjected to suction filtration and washed with water, and a product was dried to obtain 13 g of the title compound. MS (ESI) m/z (M+H)⁺=218.1.

### Step 2: Preparation of 4-hydroxy-6-(methylthio)-2-phenylpyrimidine-5-carbonitrile

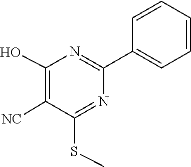 [SMILES: CSc1nc(-c2ccccc2)nc(O)c1C#N]

The ethyl 2-cyano-3,3-bis(methylthio)acrylate (13 g), benzamidine hydrochloride (7.9 g), and potassium carbonate (24.8 g) were dissolved in a mixed solution of acetonitrile and water (ACN:H₂O=4:1 (v/v)) and stirred at room temperature to react for 1 hour. When the reaction monitored by LCMS was completed, concentration was performed under reduced pressure to remove the organic solvents and precipitate a solid, which was filtrated, and a filter cake was washed with water and subjected to vacuum drying to obtain 16 g of the title compound. MS (ESI) m/z (M+H)⁺=244.1.

### Step 3: Preparation of 4-chloro-6-(methylthio)-2-phenylpyrimidine-5-carbonitrile

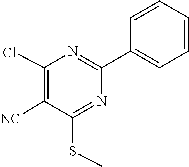 [SMILES: CSc1nc(-c2ccccc2)nc(Cl)c1C#N]

The crude 4-hydroxy-6-(methylthio)-2-phenylpyrimidine-5-carbonitrile (16 g) was dissolved in phosphorus oxychloride (100 mL), heated to 100° C., and stirred to react for 2 hours. When raw materials monitored by LCMS depleted, concentration was performed under reduced pressure to remove phosphorus oxychloride. A residue was poured into ice water, adjusted to weakly alkaline with a saturated NaHCO₃ solution, and extracted with ethyl acetate (100 mL) for three times. Organic phases were combined, dried over anhydrous sodium sulfate, filtered, and concentrated under reduced pressure, and a residue was purified by silica gel column chromatography to obtain 8 g of the title compound. MS (ESI) m/z (M+H)⁺=262.1.

### Step 4: Preparation of ethyl 5-amino-4-(methylthio)-2-phenylthieno[2,3-d]pyrimidine-6-carboxylate

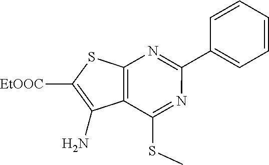 [SMILES: CCOC(=O)c1sc2nc(-c3ccccc3)nc(SC)c2c1N]

Sodium hydride (2.4 g) was dissolved in tetrahydrofuran (50 mL), into which ethyl thioglycolate (1.7 mL) was slowly added dropwise in an ice salt bath under the protection of nitrogen, and after the dropping was completed, a reaction was carried out for 0.5 hours. Then, the 4-chloro-6-(methylthio)-2-phenylpyrimidine-5-carbonitrile (2 g) was slowly added and continuously stirred under the ice salt bath to react for 1 hour. When the reaction monitored by LCMS was completed, a saturated ammonium chloride solution was added to quench the reaction, concentration was performed under reduced pressure to remove tetrahydrofuran, and a residue was extracted with ethyl acetate (50 mL) for three times. Organic phases were combined, dried over anhydrous sodium sulfate, filtered, and concentrated under reduced pressure, and a residue was purified by silica gel column chromatography to obtain 2.3 g of the title compound. MS (ESI) m/z (M+H)⁺=346.1.

### Step 5: Preparation of ethyl 5-amino-4-((2-hydroxyethyl)amino)-2-phenylthieno[2,3-d]pyrimidine-6-carboxylate

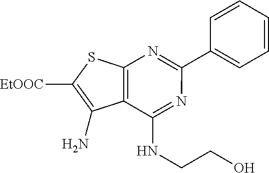 [SMILES: CCOC(=O)c1sc2nc(-c3ccccc3)nc(NCCO)c2c1N]

The ethyl 5-amino-4-(methylthio)-2-phenylthieno[2,3-d]pyrimidine-6-carboxylate (0.6 g) and 2-aminoethanol (0.4 g) were dissolved in N,N-dimethylacetamide (5 mL), and heated to 160° C. via microwave to react for 2 hours. When the reaction monitored by LCMS was completed, 20 mL of water was added, and extraction was performed with ethyl acetate (20 mL) for three times. Organic phases were combined, dried over anhydrous sodium sulfate, filtered, and concentrated under reduced pressure, and a residue was purified by silica gel column chromatography to obtain 0.5 g of the title compound. MS (ESI) m/z (M+H)⁺=359.1.

### Step 6: Preparation of 5-amino-4-((2-hydroxyethyl)amino)-2-phenylthieno[2,3-d]pyrimidine-6-carboxylic acid

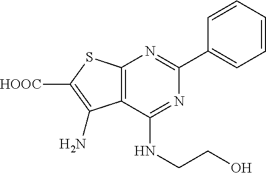 [SMILES: Nc1c(C(=O)O)sc2nc(-c3ccccc3)nc(NCCO)c12]

The ethyl 5-amino-4-((2-hydroxyethyl)amino)-2-phenylthieno[2,3-d]pyrimidine-6-carboxylate (100 mg) was dissolved in a mixed solution of ethanol and water (EtOH:H₂O=1:1 (v/v)), then lithium hydroxide monohydrate (67 mg) was added thereto, and it was raised to 100° C. to react for 0.5 hour. When the reaction monitored by LCMS was completed, concentration was performed under reduced pressure to obtain 150 mg of a crude product of the target compound, which was directly used in a next reaction without purification. MS (ESI) m/z (M+H)⁺=331.1.

### Step 7: Preparation of (5-amino-4-((2-hydroxyethyl)amino)-2-phenylthieno[2,3-d]pyrimidin-6-yl)(4-fluoropiperidin-1-yl)methanone

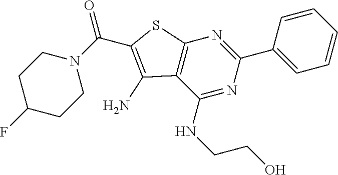 [SMILES: Nc1c(C(=O)N2CCC(F)CC2)sc2nc(-c3ccccc3)nc(NCCO)c12]

5-amino-4-((2-hydroxyethyl)amino)-2-phenylthieno[2,3-d]pyrimidine-6-carboxylic acid (100 mg), 4-fluoropiperidine hydrochloride (84 mg), and 2-(7-azabenzotriazol-1-yl)-N,N,N′,N′-tetramethyluronium hexafluorophosphate (170 mg) were dissolved in tetrahydrofuran (5 mL), into which triethylamine (0.5 mL) was added, and stirring was performed at room temperature to react for 2 hours. When the reaction monitored by LCMS was completed, 20 mL of water was added, and extraction was performed with dichloromethane (20 mL) for three times. Organic phases were combined, dried over anhydrous sodium sulfate, filtered, and concentrated under reduced pressure, and a residue was purified by silica gel column chromatography to obtain 110 mg of the title compound. MS (ESI) m/z (M+H)⁺=416.1.

### Step 8: Preparation of (9-amino-5-phenyl-2,3-dihydroimidazo[1,2-c]thieno[3,2-e]pyrimidin-8-yl)(4-fluoropiperidin-1-yl)methanone (compound 8)

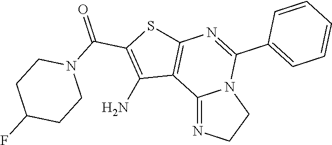 [SMILES: Nc1c(C(=O)N2CCC(F)CC2)sc2c1C1=NCCN1C(c1ccccc1)=N2]

(5-amino-4-((2-hydroxyethyl)amino)-2-phenylthieno[2,3-d]pyrimidin-6-yl)(4-fluoropiperidin-1-yl)methanone (100 mg), p-toluenesulfonyl chloride (92 mg), 4-dimethylaminopyridine (15 mg), and triethylamine (120 mg) were dissolved in dichloromethane (5 mL) and stirred at room temperature to react for 3 hours. When the reaction monitored by LCMS was completed, water (10 mL) was added to stop the reaction, liquid separation was performed, and an aqueous phase was extracted with ethyl acetate (10 mL) for three times. Organic phases were combined, dried over anhydrous sodium sulfate, filtered, and concentrated under reduced pressure, and a residue was purified by silica gel column chromatography to obtain 50 mg of the title compound. MS (ESI) m/z (M+H)⁺=398.1.

¹H NMR (400 MHz, DMSO-d₆) δ 7.75-7.73 (m, 2H), 7.65-7.46 (m, 3H), 6.41 (s, 2H), 4.99-4.83 (m, 1H), 4.04-3.99 (m, 2H), 3.94-3.89 (m, 2H), 3.68-3.62 (m, 2H), 3.57-3.51 (m, 2H), 1.99-1.87 (m, 2H), 1.80-1.71 (m, 2H).

### Step 9: Preparation of (9-amino-5-phenylimidazo[1,2-c]thieno[3,2-e]pyrimidin-8-yl)(4-fluoropiperidin-1-yl)methanone

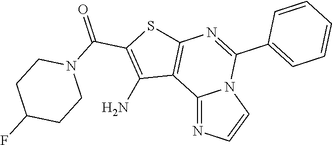 [SMILES: Nc1c(C(=O)N2CCC(F)CC2)sc2nc(-c3ccccc3)n3ccnc3c12]

(9-amino-5-phenyl-2,3-dihydroimidazo[1,2-c]thieno[3,2-e]pyrimidin-8-yl)(4-fluoropiperidin-1-yl)methanone (50 mg) and 2,3-dichloro-5,6-dicyanobenzoquinone (75 mg) were dissolved in 1,4-dioxane (5 mL), heated to 70° C., and stirred to react for 1 hour. When the reaction monitored by LCMS was completed, water (10 mL) was added to stop the reaction, and extraction was performed with ethyl acetate (10 mL) for three times. Organic phases were combined, dried over anhydrous sodium sulfate, filtered, and concentrated under reduced pressure, and a residue was purified by Prep-HPLC to obtain the title compound. MS (ESI) m/z (M+H)⁺=396.1.

¹H NMR (400 MHz, DMSO-d₆) δ 8.02 (d, J=1.6 Hz, 1H), 8.01-7.90 (m, 2H), 7.77-7.58 (m, 4H), 6.54 (s, 2H), 5.01-4.88 (m, 1H), 3.76-3.70 (m, 2H), 3.67-3.61 (m, 2H), 2.03-1.92 (m, 2H), 1.83-1.76 (m, 2H).

### Example 2: Preparation of (9-amino-5-phenylimidazo[1,2-c]thieno[3,2-e]pyrimidin-8-yl)(4-fluoropiperidin-1-yl)methanone

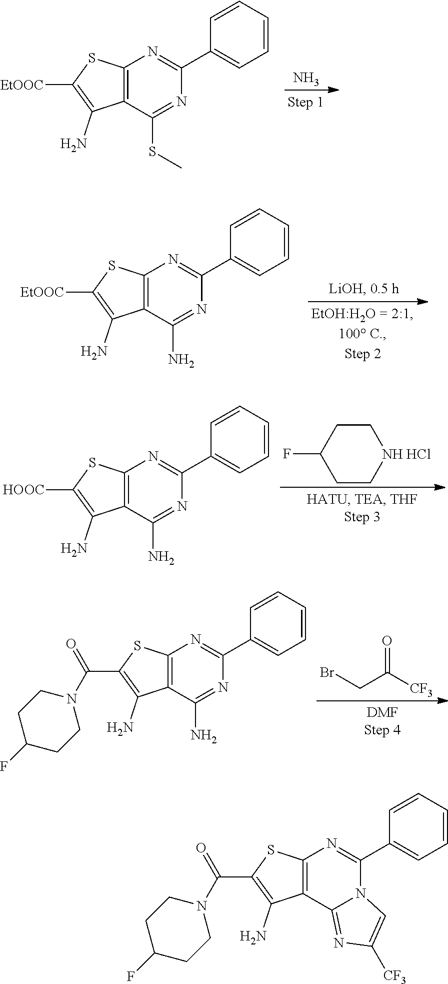

### Step 1: Preparation of ethyl 4,5-diamino-2-phenylthieno[2,3-d]pyrimidine-6-carboxylate

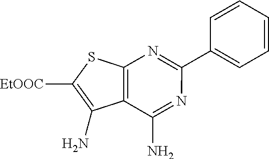 [SMILES: CCOC(=O)c1sc2nc(-c3ccccc3)nc(N)c2c1N]

Ethyl 5-amino-4-(methylthio)-2-phenylthieno[2,3-d]pyrimidine-6-carboxylate (400 mg) was added into a 7 M methanol solution of ammonia (5 mL), and heated to 100° C. via microwave to react for 6 hours. When the reaction monitored by LCMS was completed, the system was concentrated under reduced pressure, and a residue was purified by silica gel column chromatography to obtain 280 mg of the title compound. MS (ESI) m/z (M+H)⁺=315.2.

### Step 2: Preparation of 4,5-diamino-2-phenylthieno[2,3-d]pyrimidine-6-carboxylic acid

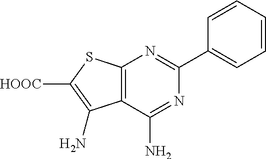 [SMILES: Nc1nc(-c2ccccc2)nc2sc(C(=O)O)c(N)c12]

Ethyl 4,5-diamino-2-phenylthieno[2,3-d]pyrimidine-6-carboxylate (280 mg) was dissolved in a mixed solution of ethanol and water (EtOH:H₂O=1:1 (v/v), 20 mL), then lithium hydroxide monohydrate (330 mg) was added thereto, and it was raised to 100° C. to react for 0.5 hours. When the reaction monitored by LCMS was completed, concentration was performed under reduced pressure to obtain 650 mg of a crude product of the target compound, which was directly used in a next reaction without purification. MS (ESI) m/z (M+H)⁺=287.2.

### Step 3: Preparation of (4,5-diamino-2-phenylthieno[2,3-d]pyrimidin-6-yl)(4-fluoropiperidin-1-yl)methanone

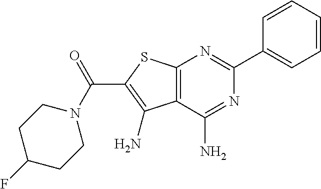 [SMILES: Nc1nc(-c2ccccc2)nc2sc(C(=O)N3CCC(F)CC3)c(N)c12]

The crude product of 4,5-diamino-2-phenylthieno[2,3-d]pyrimidine-6-carboxylic acid (650 mg) was dissolved in tetrahydrofuran (30 mL), then 2-(7-azabenzotriazol-1-yl)-N,N,N′,N′-tetramethyluronium hexafluorophosphate (800 mg), N,N-diisopropylethylamine (410 mg), and 4-fluoropiperidine hydrochloride (220 mg) were added thereto, and stirred at room temperature to react for 1 hour. When the reaction monitored by LCMS was completed, 20 mL of water was added, and extraction was performed with dichloromethane (20 mL) for three times. Organic phases were combined, dried over anhydrous sodium sulfate, filtered, and concentrated under reduced pressure, and a residue was purified by silica gel column chromatography to obtain 150 mg of the title compound. MS (ESI) m/z (M+H)⁺=372.2.

### Step 4: Preparation of (9-amino-5-phenyl-2-(trifluoromethyl)imidazo[1,2-c]thieno[3,2-e]pyrimidin-8-yl)(4-fluoropiperidin-1-yl)methanone

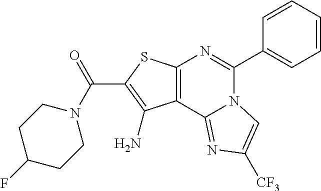 [SMILES: Nc1c(C(=O)N2CCC(F)CC2)sc2nc(-c3ccccc3)n3cc(C(F)(F)F)nc3c12]

(4,5-diamino-2-phenylthieno[2,3-d]pyrimidin-6-yl)(4-fluoropiperidin-1-yl)methanone (100 mg) was dissolved in N,N-dimethylformamide (8 mL), 3-bromo-1,1,1-trifluoroacetone (100 mg) was added thereto, and it was raised to 100° C. via microwave and stirred to react for 30 minutes. When the reaction monitored by LCMS was completed, the system was concentrated under reduced pressure, and a residue was purified by Prep-HPLC to obtain the title compound. MS (ESI) m/z (M+H)⁺=464.2.

¹H NMR (400 MHz, DMSO-d₆) δ 8.53 (s, 1H), 7.98-7.96 (m, 2H), 7.72-7.64 (m, 3H), 6.41 (s, 2H), 5.03-4.86 (m, 1H), 3.75-3.70 (m, 2H), 3.67-3.61 (m, 2H), 2.04-2.00 (m, 2H), 1.83-1.76 (m, 2H).

### Example 3: Preparation of (9-amino-5-(pyridin-4-yl)imidazo[1,2-c]thieno[3,2-e]pyrimidin-8-yl)(piperidin-1-yl)methanone

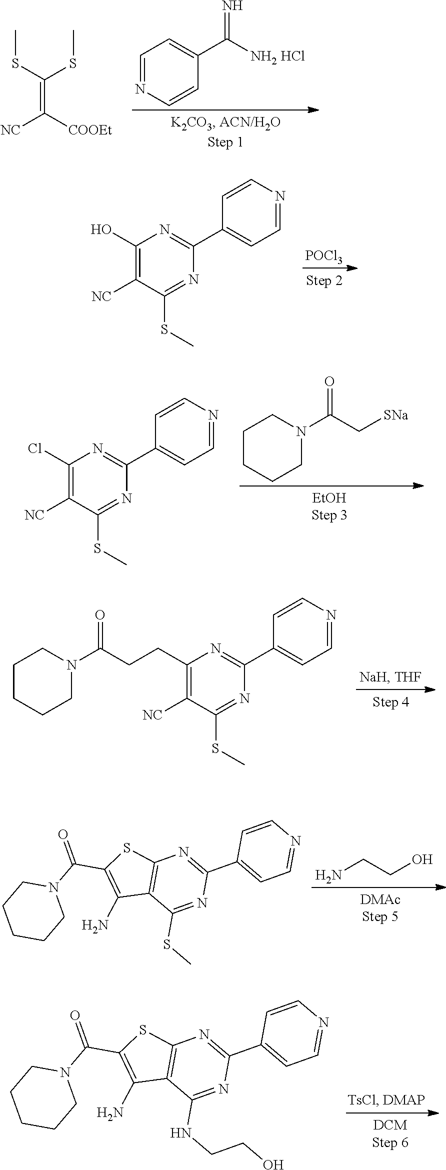

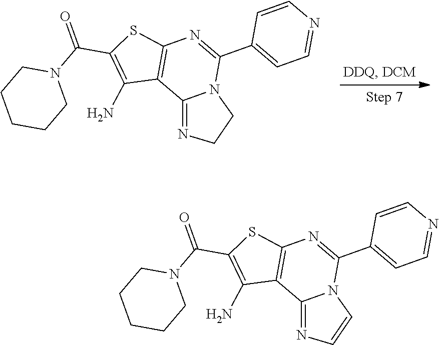 [SMILES: Nc1c(C(=O)N2CCCCC2)sc2c1C1=NCCN1C(c1ccncc1)=N2 ; Nc1c(C(=O)N2CCCCC2)sc2nc(-c3ccncc3)n3ccnc3c12]

### Step 1: Preparation of 4-hydroxy-6-(methylthio)-2-(pyridin-4-yl)pyrimidine-5-carbonitrile

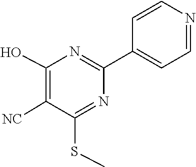 [SMILES: CSc1nc(-c2ccncc2)nc(O)c1C#N]

Ethyl 2-cyano-3,3-bis(methylthio)acrylate (600 mg), benzamidine hydrochloride (400 mg), and potassium carbonate (760 mg) were dissolved in a mixed solution of acetonitrile and water (ACN:H₂O=4:1 (v/v), 20 mL) and stirred at room temperature to react for 1 hour. When raw materials monitored by LCMS depleted, concentration was performed under reduced pressure to remove the organic solvents and precipitate a solid, filtration was performed, and a filter cake was washed with water and subjected to vacuum drying to obtain 500 mg of the title compound. MS (ESI) m/z (M+H)⁺=245.1.

### Step 2: Preparation of 4-chloro-6-(methylthio)-2-(pyridin-4-yl)pyrimidine-5-carbonitrile

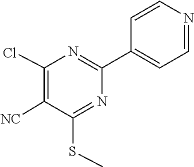 [SMILES: CSc1nc(-c2ccncc2)nc(Cl)c1C#N]

A crude 4-hydroxy-6-(methylthio)-2-(pyridin-4-yl)pyrimidine-5-carbonitrile (500 mg) was dissolved in phosphorus oxychloride (10 mL), heated to 120° C., and stirred to react for 2 hours. When the reaction monitored by LCMS was completed, concentration was performed under reduced pressure to remove phosphorus oxychloride. A residue was poured into ice water, adjusted to weakly alkaline with a saturated NaHCO₃ solution, and extracted with ethyl acetate (20 mL) for three times. Organic phases were combined, dried over anhydrous sodium sulfate, filtered, and concentrated under reduced pressure, and a residue was purified by silica gel column chromatography to obtain 400 mg of the title compound. MS (ESI) m/z (M+H)⁺=263.1.

### Step 3: Preparation of 4-(methylthio)-6-((2-oxo-2-(piperidin-1-yl)ethyl)thio)-2-(pyridin-4-yl)pyrimidine-5-carbonitrile

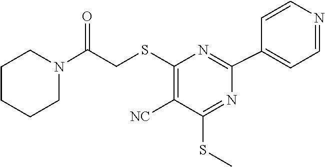 [SMILES: CSc1nc(-c2ccncc2)nc(SCC(=O)N2CCCCC2)c1C#N]

4-chloro-6-(methylthio)-2-(pyridin-4-yl)pyrimidine-5-carbonitrile (400 mg) was dissolved in ethanol (20 mL), then an ethanol-water solution of sodium 2-oxo-2-(piperidin-1-yl)ethane-1-thiolate (2.3 mL, about 1 mmoL/mL) was added thereto, and it was stirred at room temperature to react for 2 hours. When the reaction monitored by LCMS was completed, a saturated ammonium chloride solution was added to quench the reaction, concentration was performed under reduced pressure to remove ethanol, and a residue was extracted with ethyl acetate (30 mL) for three times. Organic phases were combined, dried over anhydrous sodium sulfate, filtered, and concentrated under reduced pressure, and a residue was purified by silica gel column chromatography to obtain 350 mg of the title compound. MS (ESI) m/z (M+H)⁺=386.1.

### Step 4: Preparation of (5-amino-4-(methylthio)-2-(pyridin-4-yl)thieno[2,3-d]pyrimidin-6-yl)(piperidin-1-yl)methanone

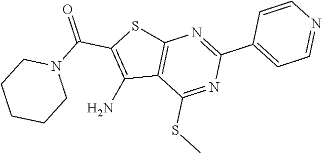 [SMILES: CSc1nc(-c2ccncc2)nc2sc(C(=O)N3CCCCC3)c(N)c12]

4-(methylthio)-6-((2-oxo-2-(piperidin-1-yl)ethyl)thio)-2-(pyridin-4-yl)pyrimidine-5-carbonitrile (350 mg) was dissolved in tetrahydrofuran (10 mL), sodium hydride (70 mg) was added thereto under the protection of nitrogen, and it was stirred at room temperature to react for 1 hour. When the reaction monitored by LCMS was completed, a saturated ammonium chloride solution was added to quench the reaction, concentration was performed under reduced pressure to remove tetrahydrofuran, and a residue was extracted with ethyl acetate (20 mL) for three times. Organic phases were combined, dried over anhydrous sodium sulfate, filtered, and concentrated under reduced pressure, and a residue was purified by silica gel column chromatography to obtain 300 mg of the title compound. MS (ESI) m/z (M+H)⁺=386.1.

### Step 5: Preparation of (5-amino-4-((2-hydroxyethyl)amino)-2-(pyridin-4-yl)thieno[2,3-d]pyrimidin-6-yl)(piperidin-1-yl)methanone

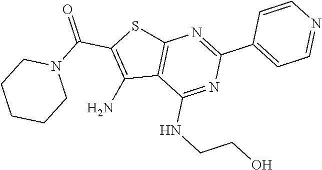 [SMILES: Nc1c(C(=O)N2CCCCC2)sc2nc(-c3ccncc3)nc(NCCO)c12]

(5-amino-4-(methylthio)-2-(pyridin-4-yl)thieno[2,3-d]pyrimidin-6-yl)(piperidin-1-yl)methanone (300 mg) and 2-aminoethanol (200 mg) were dissolved in N,N-dimethylacetamide (5 mL), and heated to 140° C. via microwave to react for 3 hours. The reaction monitored by LCMS was completed. The reaction system was directly purified by reversed phase column chromatography to obtain 200 mg of the title compound. MS (ESI) m/z (M+H)⁺=399.1.

### Step 6: Preparation of (9-amino-5-(pyridin-4-yl)-2,3-dihydroimidazo[1,2-c]thieno[3,2-e]pyrimidin-8-yl)(piperidin-1-yl)methanone

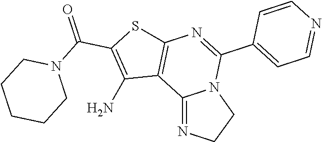 [SMILES: Nc1c(C(=O)N2CCCCC2)sc2c1C1=NCCN1C(c1ccncc1)=N2]

(5-amino-4-((2-hydroxyethyl)amino)-2-(pyridin-4-yl)thieno[2,3-d]pyrimidin-6-yl)(piperidin-1-yl)methanone (200 mg) was dissolved in dichloromethane (10 mL), then p-toluenesulfonyl chloride (170 mg) and 4-dimethylaminopyridine (200 mg) were added thereto, and it was stirred at room temperature to react overnight. When the reaction monitored by LCMS was completed, concentration was performed under reduced pressure, and a residue was purified by reversed phase column chromatography to obtain 100 mg of the title compound. MS (ESI) m/z (M+H)⁺=381.1.

### Step 7: Preparation of (9-amino-5-(pyridin-4-yl)imidazo[1,2-c]thieno[3,2-e]pyrimidin-8-yl)(piperidin-1-yl)methanone

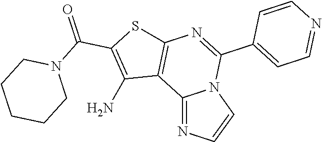 [SMILES: Nc1c(C(=O)N2CCCCC2)sc2nc(-c3ccncc3)n3ccnc3c12]

(9-amino-5-(pyridin-4-yl)-2,3-dihydroimidazo[1,2-c]thieno[3,2-e]pyrimidin-8-yl)(piperidin-1-yl)methanone (100 mg) and 2,3-dichloro-5,6-dicyanobenzoquinone (180 mg) were dissolved in dichloromethane (5 mL), and stirred at room temperature to react for 2 hours. When the reaction monitored by LCMS was completed, water (10 mL) was added to stop the reaction, and extraction was performed with dichloromethane (10 mL) for three times. Organic phases were combined, dried over anhydrous sodium sulfate, filtered, and concentrated under reduced pressure, and a residue was purified by Prep-HPLC to obtain 30 mg of the target compound. MS (ESI) m/z (M+H)⁺=379.1.

¹H NMR (400 MHz, DMSO-d₆) δ 8.91 (d, J=5.2 Hz, 2H), 8.11 (s, 1H), 7.99 (d, J=4.8 Hz, 2H), 7.73 (s, 1H), 6.46 (brs, 2H), 3.64-3.61 (m, 4H), 1.66-1.55 (m, 6H).

### Example 4: Preparation of (9-iodo-5-phenylimidazo[1,2-c]thieno[3,2-e]pyrimidin-8-yl)(piperidin-1-yl)methanone

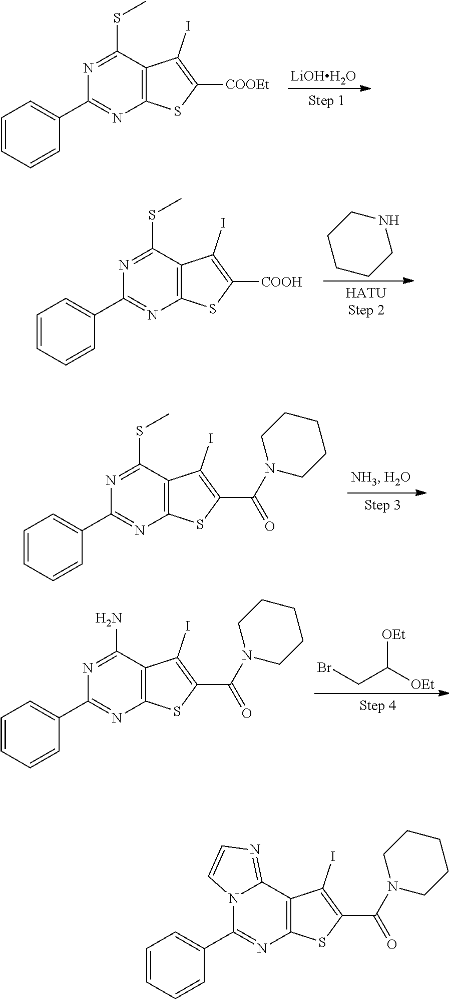

### Step 1: Preparation of 5-iodo-4-(methylthio)-2-phenylthieno[2,3-d]pyrimidine-6-carboxylic acid

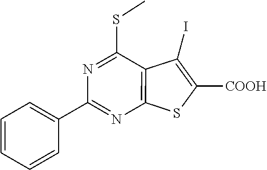 [SMILES: CSc1nc(-c2ccccc2)nc2sc(C(=O)O)c(I)c12]

Ethyl 5-iodo-4-(methylthio)-2-phenylthieno[2,3-d]pyrimidine-6-carboxylate (1.0 g, 2.2 mmol) was added into a mixed solution of ethanol and water (EtOH:H₂O=1:1 (v/v), 20 mL), then lithium hydroxide monohydrate (460 mg) was added thereto, and it was heated to 100° C. and stirred to react for 1 hour. When the reaction monitored by LCMS was completed, the reaction was stopped. The system was directly concentrated under reduced pressure, and a residue was purified by reversed phase column chromatography to obtain 700 mg of the title compound. MS (ESI) m/z (M+H)⁺=429.1.

### Step 2: Preparation of (5-iodo-4-(methylthio)-2-phenylthieno[2,3-d]pyrimidin-6-yl)(piperidin-1-yl)methanone

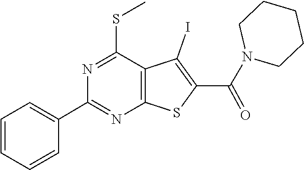 [SMILES: CSc1nc(-c2ccccc2)nc2sc(C(=O)N3CCCCC3)c(I)c12]

A crude 5-iodo-4-(methylthio)-2-phenylthieno[2,3-d]pyrimidine-6-carboxylic acid (450 mg) was dissolved in a mixed solution of tetrahydrofuran and N,N-dimethylformamide (THF:DMF=4:1 (v/v), 10 mL), then 2-(7-azabenzotriazol-1-yl)-N,N,N′,N′-tetramethyluronium hexafluorophosphate (800 mg), N,N-diisopropylethylamine (410 mg), and piperidine (180 mg) were added thereto, and it was stirred at room temperature to react for 2 hours. When LC-MS showed that the reaction of raw materials was completed, the system was concentrated under reduced pressure, and a residue was purified by silica gel column chromatography to obtain 430 mg of the title compound. MS (ESI) m/z (M+H)⁺=496.2.

### Step 3: Preparation of (4-amino-5-iodo-2-phenylthieno[2,3-d]pyrimidin-6-yl)(piperidin-1-yl)methanone

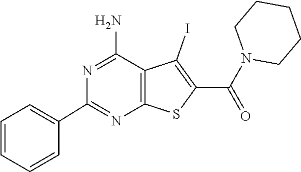 [SMILES: Nc1nc(-c2ccccc2)nc2sc(C(=O)N3CCCCC3)c(I)c12]

(5-iodo-4-(methylthio)-2-phenylthieno[2,3-d]pyrimidin-6-yl)(piperidin-1-yl)methanone (420 mg) was dissolved in ethanol (2 mL), 25% aqueous ammonia (5 mL) was added thereto, and it was stirred under microwave at 120° C. to react for 4 hours. LCMS showed that the reaction was completed. The system was concentrated under reduced pressure, and a residue was purified by silica gel column chromatography to obtain 150 mg of a crude product of the title compound. MS (ESI) m/z (M+H)⁺=465.2.

### Step 4: Preparation of (9-iodo-5-phenylimidazo[1,2-c]thieno[3,2-e]pyrimidin-8-yl)(piperidin-1-yl)methanone

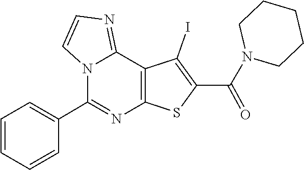 [SMILES: O=C(c1sc2nc(-c3ccccc3)n3ccnc3c2c1I)N1CCCCC1]

(4-amino-5-iodo-2-phenylthieno[2,3-d]pyrimidin-6-yl)(piperidin-1-yl)methanone (150 mg) was dissolved in acetonitrile (8 mL), 2-bromo-1,1-diethoxyethane (252 mg) was added thereto, and then it was raised to 120° C. via microwave and stirred to react for 3 hours. LCMS showed that the reaction was completed. The system was concentrated under reduced pressure, and a residue was purified by Prep-HPLC to obtain 30 mg of the target compound. MS (ESI) m/z (M+H)⁺=489.2.

¹H NMR (400 MHz, DMSO-d₆) δ 8.03 (d, J=1.6 Hz, 1H), 7.98-7.94 (m, 2H), 7.73 (d, J=1.6 Hz, 1H), 7.70-7.63 (m, 3H), 3.66 (brs, 4H), 1.73-1.51 (m, 6H).

### Example 5: Preparation of (9-amino-5-phenylthieno[3,2-e][1,2,4]triazolo[4,3-c]pyrimidin-8-yl)(piperidin-1-yl)methanone

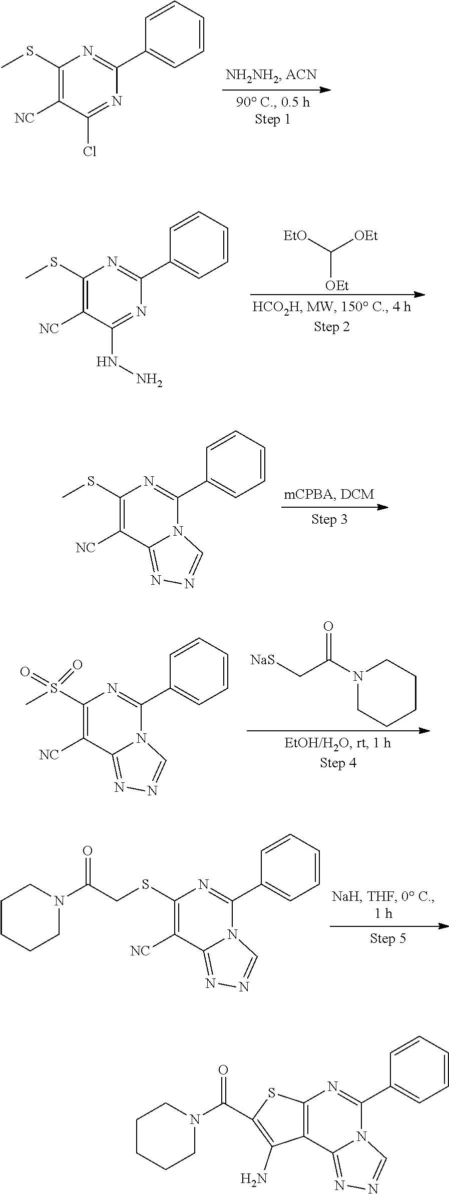

### Step 1: Preparation of 4-hydrazinyl-6-(methylthio)-2-phenylpyrimidine-5-carbonitrile

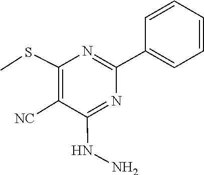 [SMILES: CSc1nc(-c2ccccc2)nc(NN)c1C#N]

4-chloro-6-(methylthio)-2-phenylpyrimidine-5-carbonitrile (0.5 g) was dissolved in acetonitrile (30 mL), hydrazine hydrate (0.93 mL) was added thereto, and it was raised to 90° C. to react for 0.5 hour. When the reaction monitored by LCMS was completed, concentration was performed under reduced pressure to remove acetonitrile, water was added to quench the reaction, and extraction was performed with ethyl acetate (8 mL) for three times. Organic phases were combined, dried over anhydrous sodium sulfate, filtered, and concentrated under reduced pressure, and a residue was purified by silica gel column chromatography to obtain 0.26 g of the title compound. MS (ESI) m/z (M+H)⁺=258.1.

### Step 2: Preparation of 7-(methylthio)-5-phenyl-[1,2,4]triazolo[4,3-c]pyrimidine-8-carbonitrile

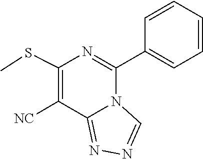 [SMILES: CSc1nc(-c2ccccc2)n2cnnc2c1C#N]

4-hydrazinyl-6-(methylthio)-2-phenylpyrimidine-5-carbonitrile (0.26 g) was dissolved in a microwave tube with triethyl orthoformate (8 mL), formic acid (0.1 mL) was added thereto, and it was raised to 150° C. via microwave and stirred to react for 4 hours. When the reaction monitored by LCMS was completed, a saturated ammonium chloride solution was added to quench the reaction, and extraction was performed with ethyl acetate (10 mL) for three times. Organic phases were combined, dried over anhydrous sodium sulfate, filtered, and concentrated under reduced pressure, and a residue was purified by silica gel column chromatography to obtain 0.16 g of the title compound. MS (ESI) m/z (M+H)⁺=268.1.

### Step 3: Preparation of 7-(methylsulfonyl)-5-phenyl-[1,2,4]triazolo[4,3-c]pyrimidine-8-carbonitrile

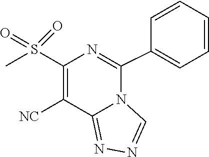 [SMILES: CS(=O)(=O)c1nc(-c2ccccc2)n2cnnc2c1C#N]

7-(methylthio)-5-phenyl-[1,2,4]triazolo[4,3-c]pyrimidine-8-carbonitrile (0.16 g) was dissolved in dichloromethane (6 mL), m-chloroperoxybenzoic acid (0.2 g) was added thereto, and it was stirred at room temperature to react for 1 hour. When the reaction monitored by LCMS was completed, a saturated ammonium chloride solution was added to quench the reaction, and extraction was performed with ethyl acetate (10 mL) for three times. Organic phases were combined, dried over anhydrous sodium sulfate, filtered, and concentrated under reduced pressure, and a residue was purified by silica gel column chromatography to obtain 0.1 g of the title compound. MS (ESI) m/z (M+H)⁺=300.1.

### Step 4: Preparation of 7-((2-oxo-2-(piperidin-1-yl)ethyl)thio)-5-phenyl-[1,2,4]triazolo[4,3-c]pyrimidine-8-carbonitrile

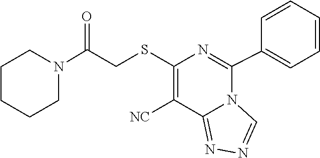 [SMILES: N#Cc1c(SCC(=O)N2CCCCC2)nc(-c2ccccc2)n2cnnc12]

7-(methylsulfonyl)-5-phenyl-[1,2,4]triazolo[4,3-c]pyrimidine-8-carbonitrile (0.1 g) was dissolved in ethanol (2 mL), then an ethanol-water solution of sodium 2-oxo-2-(piperidin-1-yl)ethane-1-thiolate (1.3 mL, about 1 mmoL/mL) was added thereto, and it was stirred at room temperature to react for 2 hours. When the reaction monitored by LCMS was completed, a saturated ammonium chloride solution was added to quench the reaction, concentration was performed under reduced pressure to remove ethanol, and a residue was extracted with ethyl acetate (30 mL) for three times. Organic phases were combined, dried over anhydrous sodium sulfate, filtered, and concentrated under reduced pressure, and a residue was purified by silica gel column chromatography to obtain 50 mg of the title compound. MS (ESI) m/z (M+H)⁺=379.1.

### Step 5: Preparation of (9-amino-5-phenylthieno[3,2-e][1,2,4]triazolo[4,3-c]pyrimidin-8-yl)(piperidin-1-yl)methanone

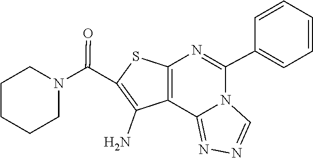 [SMILES: Nc1c(C(=O)N2CCCCC2)sc2nc(-c3ccccc3)n3cnnc3c12]

7-((2-oxo-2-(piperidin-1-yl)ethyl)thio)-5-phenyl-[1,2,4]triazolo[4,3-c]pyrimidine-8-carbonitrile (50 mg) was dissolved in tetrahydrofuran (10 mL), sodium hydride (62 mg) was added thereto under the protection of nitrogen, and it was stirred under an ice salt bath to react for 1 hour. When the reaction monitored by LCMS was completed, a saturated ammonium chloride solution was added to quench the reaction, concentration was performed under reduced pressure to remove tetrahydrofuran, and a residue was extracted with ethyl acetate (5 mL) for three times. Organic phases were combined, dried over anhydrous sodium sulfate, filtered, and concentrated under reduced pressure, and a residue was purified by Prep-HPLC to obtain 7 mg of the title compound. MS (ESI) m/z (M+H)⁺=379.1.

¹H NMR (400 MHz, DMSO-d₆) δ 8.81 (s, 1H), 8.49 (d, J=7.5 Hz, 2H), 7.69-7.62 (m, 3H), 6.33 (s, 2H), 3.61 (t, J=5.2 Hz, 4H), 1.74-1.51 (m, 6H).

### Example 6: Preparation of (9-methoxy-5-phenylimidazo[1,2-c]thieno[3,2-e]pyrimidin-8-yl)(piperidin-1-yl)methanone

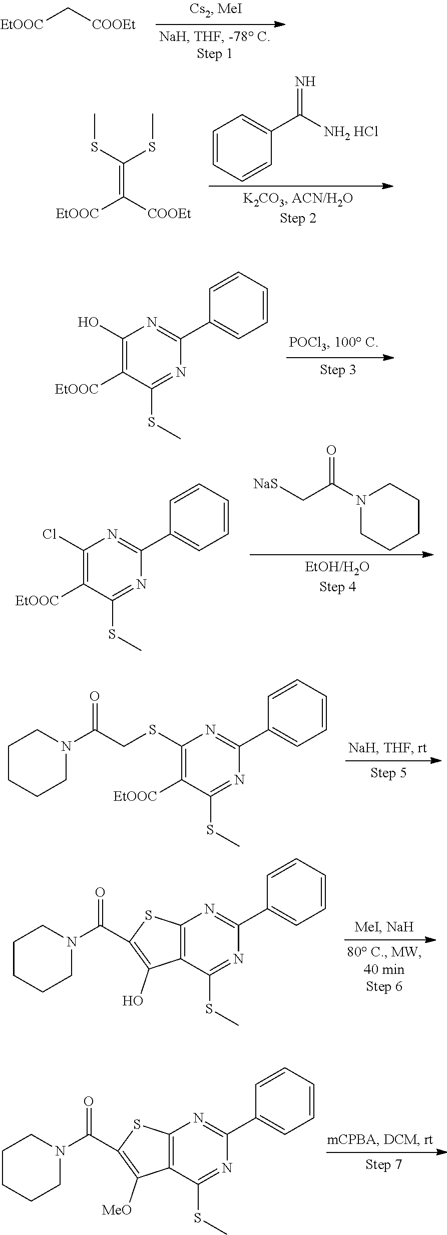

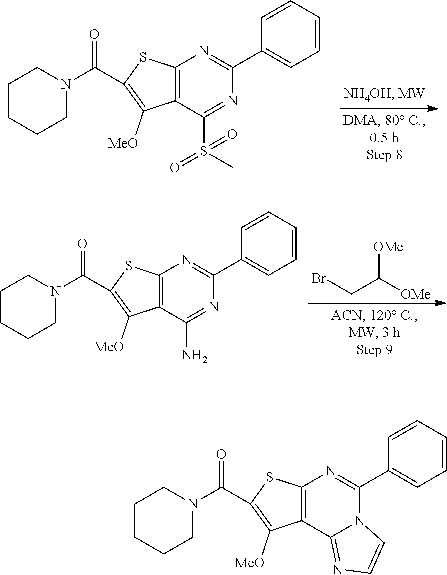

### Step 1: Preparation of diethyl 2-(bis(methylthio)methylene)malonate

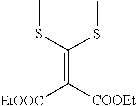 [SMILES: CCOC(=O)C(C(=O)OCC)=C(SC)SC]

60% sodium hydride (1.5 g) was added into tetrahydrofuran (30 mL), diethyl malonate (5.0 g) was added thereto under the protection of nitrogen, into which carbon disulfide (1.87 mL) was then slowly added dropwise under a dry ice bath for cooling to −78° C., and after the dropping was completed, stirring was performed continuously at −78° C. to react for 1 hour. When the system became light yellow, iodomethane (4.28 mL) was slowly added dropwise, after the dropping was completed, stirring was performed continuously at −78° C. to react for 1 hour, and then it was slowly raised to 0° C. to react for 1 hour. When the reaction monitored by LCMS was completed, water was added to quench the reaction, and concentration was performed under reduced pressure to remove tetrahydrofuran. A residue was poured into ice water and stirred continuously to precipitate a yellow solid, the solid was subjected to suction filtration and washed with water, and a product was dried to obtain 6 g of the title compound. MS (ESI) m/z (M+H)⁺=265.1.

### Step 2: Preparation of ethyl 4-hydroxy-6-(methylthio)-2-phenylpyrimidine-5-carboxylate

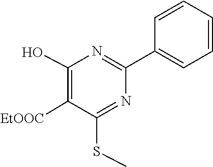 [SMILES: CCOC(=O)c1c(O)nc(-c2ccccc2)nc1SC]

Diethyl 2-(bis(methylthio)methylene)malonate (6.0 g), benzamidine hydrochloride (3.0 g), and potassium carbonate (9.4 g) were dissolved in a mixed solution of acetonitrile and water (ACN:H₂O=4:1 (v/v), 40 mL) and stirred at room temperature to react for 1 hour. When the reaction monitored by LCMS was completed, concentration was performed under reduced pressure to remove an organic solvent and precipitate a solid, filtration was performed, and a filter cake was washed with water and subjected to vacuum drying to obtain 5 g of the title compound. MS (ESI) m/z (M+H)⁺=291.1.

### Step 3: Preparation of ethyl 4-chloro-6-(methylthio)-2-phenylpyrimidine-5-carboxylate

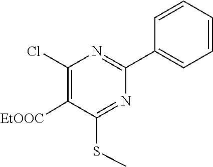 [SMILES: CCOC(=O)c1c(Cl)nc(-c2ccccc2)nc1SC]

Ethyl 4-hydroxy-6-(methylthio)-2-phenylpyrimidine-5-carboxylate (5 g) was added into phosphorus oxychloride (50 mL), heated to 100° C., and stirred to react for 2 hours. When the reaction monitored by LCMS was completed, concentration was performed under reduced pressure to remove phosphorus oxychloride. A residue was poured into ice water, adjusted to weakly alkaline with a saturated NaHCO₃ solution, and extracted with ethyl acetate (100 mL) for three times. Organic phases were combined, dried over anhydrous sodium sulfate, filtered, and concentrated under reduced pressure, and a residue was purified by silica gel column chromatography to obtain 1.6 g of the title compound. MS (ESI) m/z (M+H)⁺=309.1.

### Step 4: Preparation of ethyl 4-(methylthio)-6-((2-oxo-2-(piperidin-1-yl)ethyl)thio)-2-phenylpyrimidine-5-carboxylate

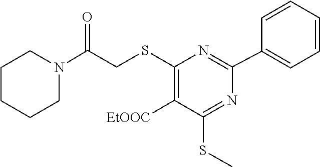 [SMILES: CCOC(=O)c1c(SC)nc(-c2ccccc2)nc1SCC(=O)N1CCCCC1]

Ethyl 4-chloro-6-(methylthio)-2-phenylpyrimidine-5-carboxylate (1.6 g) was dissolved in ethanol (40 mL), then an ethanol-water solution of sodium 2-oxo-2-(piperidin-1-yl)ethane-1-thiolate (6.5 mL, about 1 mmoL/mL) was added thereto, and it was stirred at room temperature to react for 2 hours. When the reaction monitored by LCMS was completed, a saturated ammonium chloride solution was added to quench the reaction, concentration was performed under reduced pressure to remove ethanol, and a residue was extracted with ethyl acetate (30 mL) for three times. Organic phases were combined, dried over anhydrous sodium sulfate, filtered, and concentrated under reduced pressure, and a residue was purified by silica gel column chromatography to obtain 2 g of the title compound. MS (ESI) m/z (M+H)⁺=432.1.

### Step 5: Preparation of (5-hydroxy-4-(methylthio)-2-phenylthieno[2,3-d]pyrimidin-6-yl)(piperidin-1-yl)methanone

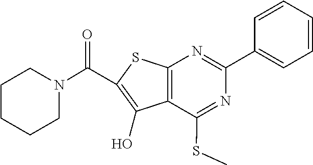 [SMILES: CSc1nc(-c2ccccc2)nc2sc(C(=O)N3CCCCC3)c(O)c12]

Ethyl 4-(methylthio)-6-((2-oxo-2-(piperidin-1-yl)ethyl)thio)-2-phenylpyrimidine-5-carboxylate (2.0 g) was dissolved in tetrahydrofuran (40 mL), 60% sodium hydride (0.33 g) was added thereto under the protection of nitrogen, and it was stirred under an ice salt bath to react 1 hour. When the reaction monitored by LCMS was completed, a saturated ammonium chloride solution was added to quench the reaction, concentration was performed under reduced pressure to remove tetrahydrofuran, and a residue was extracted with ethyl acetate (20 mL) for three times. Organic phases were combined, dried over anhydrous sodium sulfate, filtered, and concentrated under reduced pressure, and a residue was purified by silica gel column chromatography to obtain 1.8 g of the title compound. MS (ESI) m/z (M+H)⁺=386.1.

### Step 6: Preparation of (5-methoxy-4-(methylthio)-2-phenylthieno[2,3-d]pyrimidin-6-yl)(piperidin-1-yl)methanone

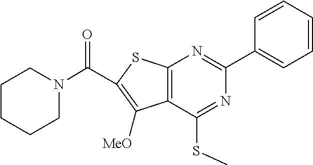 [SMILES: COc1c(C(=O)N2CCCCC2)sc2nc(-c3ccccc3)nc(SC)c12]

(5-hydroxy-4-(methylthio)-2-phenylthieno[2,3-d]pyrimidin-6-yl)(piperidin-1-yl)methanone (0.4 g) was dissolved in tetrahydrofuran (8 mL), iodomethane (0.74 g) and 60% sodium hydride (0.12 g) were added thereto, and then it was raised to 80° C. via microwave and stirred to react for 40 minutes. When the reaction monitored by LCMS was completed, a saturated ammonium chloride solution was added to quench the reaction, and extraction was performed with ethyl acetate (10 mL) for three times. Organic phases were combined, dried over anhydrous sodium sulfate, filtered, and concentrated under reduced pressure, and a residue was purified by silica gel column chromatography to obtain 0.36 g of the title compound. MS (ESI) m/z (M+H)⁺=400.1.

### Step 7: Preparation of (5-methoxy-4-(methylsulfonyl)-2-phenylthieno[2,3-d]pyrimidin-6-yl)(piperidin-1-yl)methanone

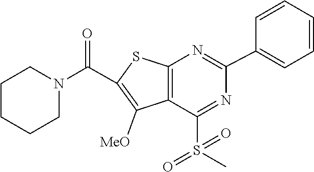 [SMILES: COc1c(C(=O)N2CCCCC2)sc2nc(-c3ccccc3)nc(S(C)(=O)=O)c12]

(5-methoxy-4-(methylthio)-2-phenylthieno[2,3-d]pyrimidin-6-yl)(piperidin-1-yl)methanone (0.36 g) and m-chloroperoxybenzoic acid (0.62 g) were dissolved in dichloromethane (8 mL) and stirred at room temperature to react for 1 hour. When the reaction monitored by LCMS was completed, a saturated ammonium chloride solution was added to quench the reaction, and extraction was performed with ethyl acetate (5 mL) for three times. Organic phases were combined, dried over anhydrous sodium sulfate, filtered, and concentrated under reduced pressure, and a residue was purified by silica gel column chromatography to obtain 0.2 g of the title compound. MS (ESI) m/z (M+H)⁺=432.1.

### Step 8: Preparation of (4-amino-5-methoxy-2-phenylthieno[2,3-d]pyrimidin-6-yl)(piperidin-1-yl)methanone

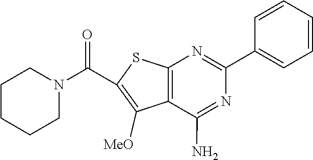 [SMILES: COc1c(C(=O)N2CCCCC2)sc2nc(-c3ccccc3)nc(N)c12]

(5-methoxy-4-(methylsulfonyl)-2-phenylthieno[2,3-d]pyrimidin-6-yl)(piperidin-1-yl)methanone (0.39 g) was dissolved in N,N-dimethylacetamide (8 mL), 25% aqueous ammonia (0.5 mL) was added thereto, and it was raised to 80° C. via microwave and stirred to react for 30 minutes. When the reaction monitored by LCMS was completed, a saturated ammonium chloride solution was added to quench the reaction, and extraction was performed with ethyl acetate (10 mL) for three times. Organic phases were combined, dried over anhydrous sodium sulfate, filtered, and concentrated under reduced pressure, and a residue was purified by silica gel column chromatography to obtain 0.25 g of the title compound. MS (ESI) m/z (M+H)⁺=369.1.

### Step 9: Preparation of (9-methoxy-5-phenylimidazo[1,2-c]thieno[3,2-e]pyrimidin-8-yl)(piperidin-1-yl)methanone

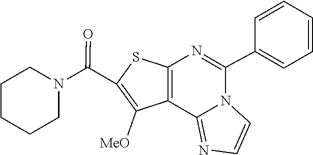 [SMILES: COc1c(C(=O)N2CCCCC2)sc2nc(-c3ccccc3)n3ccnc3c12]

(4-amino-5-methoxy-2-phenylthieno[2,3-d]pyrimidin-6-yl)(piperidin-1-yl)methanone (60 mg) was dissolved in acetonitrile (8 mL), 2-bromo-1,1-dimethoxyethane (0.27 g) was added thereto, and it was raised to 120° C. via microwave to react for 3 hours. When the reaction monitored by LCMS was completed, a saturated ammonium chloride solution was added to quench the reaction, and extraction was performed with ethyl acetate (5 mL) for three times. Organic phases were combined, dried over anhydrous sodium sulfate, filtered, and concentrated under reduced pressure, and a residue was purified by Prep-HPLC to obtain 10 mg of the title compound. MS (ESI) m/z (M+H)⁺=393.1.

¹H NMR (400 MHz, DMSO-d₆) δ 8.01 (d, J=1.6 Hz, 1H), 7.97-7.94 (m, 2H), 7.74-7.61 (m, 41H), 4.16 (s, 3H), 3.57 (brs, 4H), 1.66-1.57 (n, 6).

### Examples 7-21

A series of compounds were prepared from the corresponding commercial reagents and the products in the foregoing preparation examples and Examples as raw materials, by using the preparation methods similar to that of the foregoing Examples, and the structures and characterization data of said compounds are shown in Table 1:

TABLE 1

| | | Reference | |
|---|---|---|---|
| | | Examples of | |
| Ex- | | Preparation | |
| ample | Structure | Methods | MS(M + H)⁺ and ¹H NMR |
| 7 | 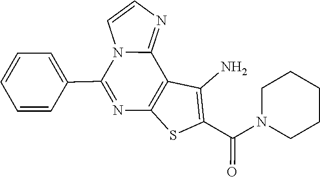 [SMILES: Nc1c(C(=O)N2CCCCC2)sc2nc(-c3ccccc3)n3ccnc3c12] | Example 1 | MS (ESI) m/z (M + H)⁺ = 378.1, ¹H NMR (400 MHz, DMSO-d₆) δ 8.01 (d, J = 1.6 Hz, 1H), 8.00-7.92 (m, 2H), 7.75-7.59 (m, 4H), 6.49 (s, 2H), 3.72-3.49 (m, 4H), 1.73-1.48 (m, 6H) |
| 8 | 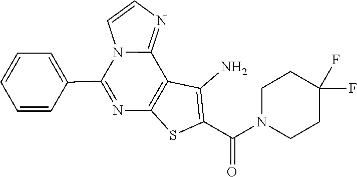 [SMILES: Nc1c(C(=O)N2CCC(F)(F)CC2)sc2nc(-c3ccccc3)n3ccnc3c12] | Example 1 | MS (ESI) m/z (M + H)⁺ = 414.1, ¹H NMR (400 MHz, DMSO-d₆) δ 8.01 (d, J = 1.6 Hz, 1H), 8.01-7.95 (m, 2H), 7.71-7.64 (m, 4H), 6.56 (s, 2H), 3.74-3.71 (m, 4H), 2.14-2.06 (m, 4H) |
| 9 | 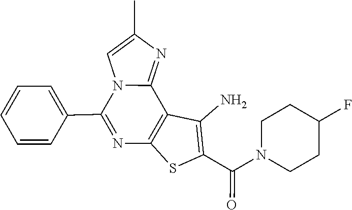 [SMILES: Cc1cn2c(-c3ccccc3)nc3sc(C(=O)N4CCC(F)CC4)c(N)c3c2n1] | Example 2 | MS (ESI) m/z (M + H)⁺ = 410.1, ¹H NMR (400 MHz, DMSO-d₆) δ 7.96-7.94 (m, 2H), 7.79 (s, 1H), 7.71-7.63 (m, 3H), 6.52 (s, 2H), 5.02-4.87 (m, 1H), 3.76-3.70 (m, 2H), 3.66-3.60 (m, 2H), 2.40 (s, 3H), 2.04-1.91 (m, 2H), 1.83- 1.77 (m, 2H) |
| 10 | 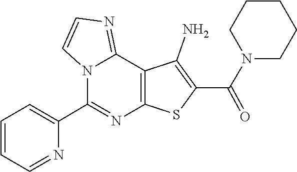 [SMILES: Nc1c(C(=O)N2CCCCC2)sc2nc(-c3ccccn3)n3ccnc3c12] | Example 3 | MS (ESI) m/z (M + H)⁺ = 379.1, ¹H NMR (400 MHz, DMSO-d₆) δ 9.26 (d, J = 1.7 Hz, 1H), 8.89 (d, J = 4.8 Hz, 1H), 8.52 (d, J = 8.1 Hz, 1H), 8.14 (td, J = 7.8, 1.9 Hz, 1H), 7.77 (d, J = 1.7 Hz, 1H), 7.71 (dd, J = 7.6, 5.0 Hz, 1H), 6.53 (s, 2H), 3.62 (t, J = 5.3 Hz, 4H), 1.73-1.52 (m, 6H). |
| 11 | 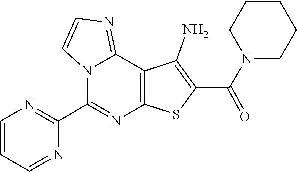 [SMILES: Nc1c(C(=O)N2CCCCC2)sc2nc(-c3ncccn3)n3ccnc3c12] | Example 3 | MS (ESI) m/z (M + H)⁺ = 379.1, ¹H NMR (400 MHz, DMSO-d₆) δ 9.17 (d, J = 4.9 Hz, 2H), 8.88 (d, J = 1.8 Hz, 1H), 7.81 (t, J = 4.9 Hz, 1H) |
| 12 | 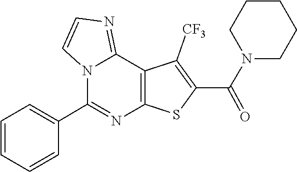 [SMILES: O=C(c1sc2nc(-c3ccccc3)n3ccnc3c2c1C(F)(F)F)N1CCCCC1] | Example 4 | MS (ESI) m/z (M + H)⁺ = 431.1, ¹H NMR (400 MHz, DMSO-d₆) δ 8.07 (s, 1H), 7.98 (d, J = 7.3 Hz, 2H), 7.75 (s, 1H), 7.72-7.67 (m, 3H), 3.66 (brs, 2H), 3.30 (t, J = 5.6 Hz, 2H), 1.68-1.45 (m, 6H) |
| 13 | 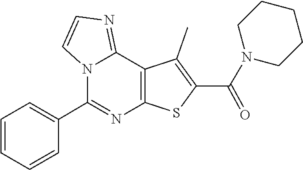 [SMILES: Cc1c(C(=O)N2CCCCC2)sc2nc(-c3ccccc3)n3ccnc3c12] | Example 4 | MS (ESI) m/z (M + H)⁺ = 377.1, ¹H NMR (400 MHz, DMSO-d₆) δ 8.02 (s, 1H), 7.96 (d, J = 6.9 Hz, 2H), 7.76-7.61 (m, 4H), 3.53 (brs, 4H), 2.71 (s, 3H), 1.64-1.54 (m, 6H) |
| 14 | 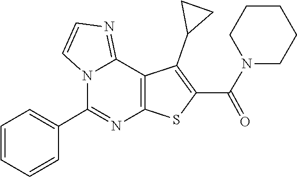 [SMILES: O=C(c1sc2nc(-c3ccccc3)n3ccnc3c2c1C1CC1)N1CCCCC1] | Example 4 | MS (ESI) m/z (M + H)⁺ = 403.1, ¹H NMR (400 MHz, DMSO-d₆) δ 8.00 (s, 1H), 7.95 (d, J = 7.0 Hz, 2H), 7.73-7.62 (m, 4H), 3.65 (brs, 2H), 3.39 (brs, 2H), 2.64-2.56 (m, 1H), 1.68- 1.48 (m, 6H), 1.09-1.00 (m, 2H), 0.90-0.86 (m, 2H) |
| 15 | 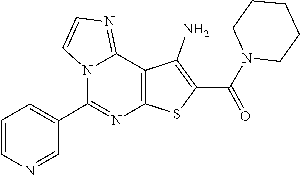 [SMILES: Nc1c(C(=O)N2CCCCC2)sc2nc(-c3cccnc3)n3ccnc3c12] | Example 3 | MS (ESI) m/z (M + H)⁺ = 379.1, ¹H NMR (400 MHz, DMSO-d₆) δ 9.12 (d, J = 2.2 Hz, 1H), 8.85 (d, J = 4.9 Hz, 1H), 8.45-8.35 (m, 1H), 8.07 (s, 1H), 7.78-7.63 (m, 2H), 6.49 (s, 2H), 3.61 (t, J = 5.2 Hz, 4H), 1.78-1.45 (m, 6H) |
| 16 | 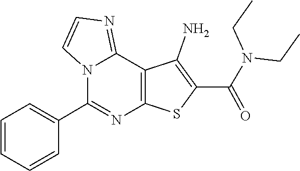 [SMILES: CCN(CC)C(=O)c1sc2nc(-c3ccccc3)n3ccnc3c2c1N] | Example 1 | MS (ESI) m/z (M + H)⁺ = 366.1, ¹H NMR (400 MHz, DMSO-d₆) δ 8.02 (d, J = 1.6 Hz, 1H), 8.01-7.95 (m, 2H), 7.70-7.66 (m, 4H), 6.84 (s, 2H), 3.56 (q, J = 7.0 Hz, 4H), 1.22 (t, J = 7.0 Hz, 6H) |
| 17 | 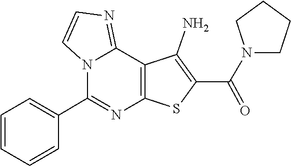 [SMILES: Nc1c(C(=O)N2CCCC2)sc2nc(-c3ccccc3)n3ccnc3c12] | Example 1 | MS (ESI) m/z (M + H)⁺ = 364.1, ¹H NMR (400 MHz, DMSO-d₆) δ 8.02 (d, J = 1.7 Hz, 1H), 7.98-795 (m, 2H), 7.74-7.62 (m, 4H), 7.16 (s, 2H), 3.67 (brs, 4H), 1.93 (brs, 4H) |
| 18 | 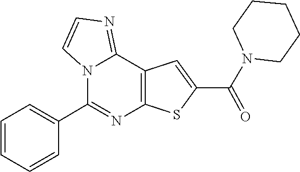 [SMILES: O=C(c1cc2c(nc(-c3ccccc3)n3ccnc23)s1)N1CCCCC1] | Example 4 | MS (ESI) m/z (M + H)⁺ = 363.1, ¹H NMR (400 MHz, DMSO-d₆) δ 8.01 (s, 1H), 7.97 (d, J = 7.3 Hz, 2H), 7.94 (s, 1H), 7.71-7.64 (m, 4H), 3.68 (t, J = 5.2 Hz, 4H), 1.68-1.59 (m, 6H) |
| 19 | 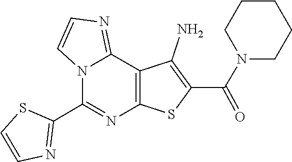 [SMILES: Nc1c(C(=O)N2CCCCC2)sc2nc(-c3nccs3)n3ccnc3c12] | Example 3 | MS (ESI) m/z (M + H)⁺ = 385.1, ¹H NMR (400 MHz, DMSO-d₆) δ 9.32 (s, 1H), 8.30 (d, J = 3.2 Hz, 1H), 8.24 (d, J = 3.4 Hz, 1H), 7.84 (s, 1H), 6.51 (s, 2H), 3.62 (t, J = 5.3 Hz, 4H), 1.66-1.58 (m, 6H) |
| 20 | 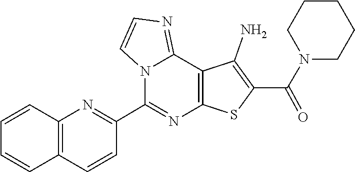 [SMILES: Nc1c(C(=O)N2CCCCC2)sc2nc(-c3ccc4ccccc4n3)n3ccnc3c12] | Example 3 | MS (ESI) m/z (M + H)⁺ = 429.1, ¹H NMR (400 MHz, DMSO-d₆) δ 9.76 (s, 1H), 8.68 (s, 2H), 8.37 (d, J = 8.4 Hz, 1H), 8.16 (d, J = 8.0 Hz, 1H), 7.95 (t, J = 8.0 Hz, 1H), 7.86 (s, 1H), 7.80 (t, J = 7.6 Hz, 1H), 6.56 (s, 2H), 3.64 (t, J = 5.2 Hz, 4H), 1.66-1.60 (m, 6H) |
| 21 | 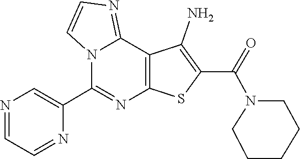 [SMILES: Nc1c(C(=O)N2CCCCC2)sc2nc(-c3cnccn3)n3ccnc3c12] | Example 3 | MS (ESI) m/z (M + H)⁺ = 380.12, ¹H NMR (400 MHz, DMSO-d₆) δ 9.66 (s, 1H), 9.13 (d, J = 1.5 Hz, 1H), 8.97-8.94 (m, 1H), 8.93 (d, J = 2.5 Hz, 1H), 7.80 (s, 1H), 6.53 (s, 2H), 3.69-3.55 (m, 4H), 1.69-1.63 (m, 2H), 1.63-1.48 (m, 4H) |

### Example 22: Preparation of (9-iodo-5-phenylimidazo[1,2-c]thieno[3,2-e]pyrimidin-8-yl)(piperidin-1-yl)methanone

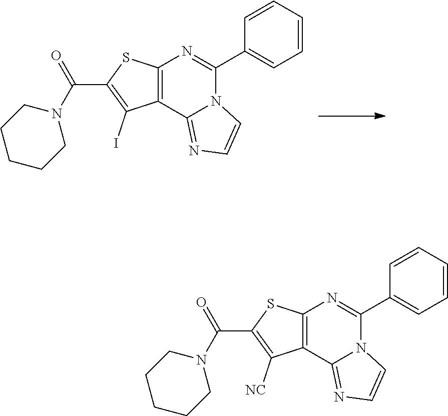 [SMILES: O=C(c1sc2nc(-c3ccccc3)n3ccnc3c2c1I)N1CCCCC1 ; N#Cc1c(C(=O)N2CCCCC2)sc2nc(-c3ccccc3)n3ccnc3c12]

(9-iodo-5-phenylimidazo[1,2-c]thieno[3,2-e]pyrimidin-8-yl)(piperidin-1-yl)methanone (15.0 mg) was dissolved in N,N-dimethylformamide (3 mL), cuprous iodide (6.0 mg) and cuprous cyanide (5.5 mg) were added thereto, after nitrogen replacement for protection, it was raised to 90° C. and stirred to react for 2 hours. When the reaction monitored by LCMS was completed, the system was concentrated under reduced pressure, and a residue was purified by Prep-HPLC to obtain 3 mg of the title compound.

MS (ESI) m/z (M+H)⁺=388.1.

¹H NMR (400 MHz, DMSO-d₆) δ 8.13 (d, J=1.5 Hz, 1H), 8.03-7.94 (m, 2H), 7.79 (s, 1H), 7.70 (dq, J=14.0, 6.9 Hz, 3H), 3.59 (s, 4H), 1.64 (d, J=20.2 Hz, 6H).

### Example 23: Preparation of (9-isopropyl-5-phenylimidazo[1,2-c]thieno[3,2-e]pyrimidin-8-yl)(piperidin-1-yl)methanone

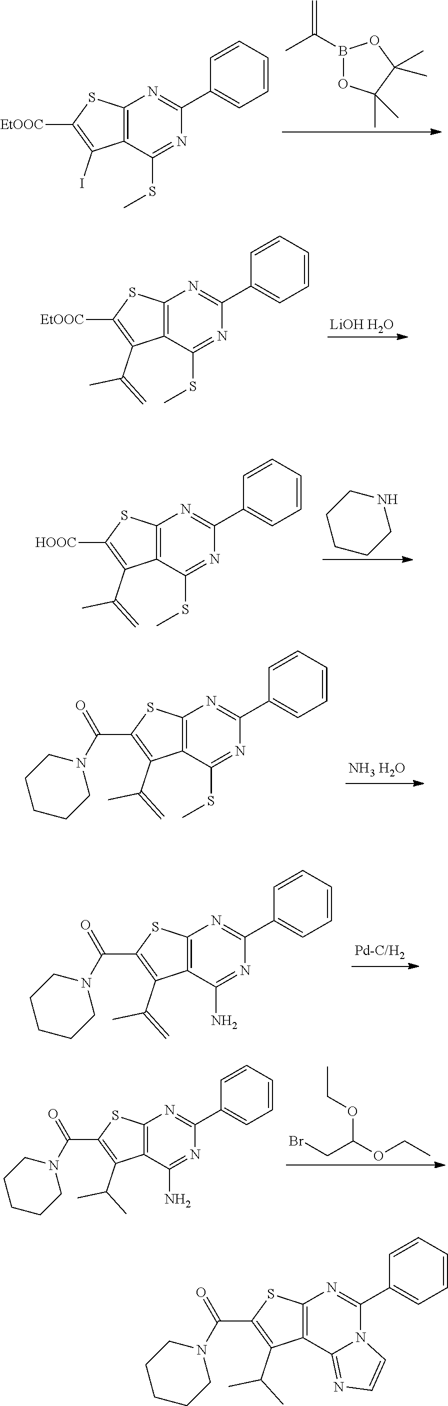

### Step 1: Preparation of ethyl 4-(methylthio)-2-phenyl-5-(propyl-1-ene-2-yl)thieno[2,3-d]pyrimidine-6-carboxylate

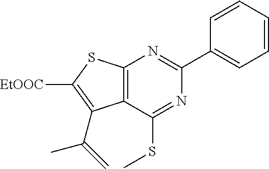 [SMILES: C=C(C)c1c(C(=O)OCC)sc2nc(-c3ccccc3)nc(SC)c12]

Ethyl 5-iodo-4-(methylthio)-2-phenylthieno[2,3-d]pyrimidine-6-carboxylate (300 mg) was dissolved in a mixed solution of dioxane and water (dioxane:H₂O=4:1), and then potassium carbonate (182 mg), [1,1′-bis(diphenylphosphino)ferrocene]dichloropalladium (48 mg), and isopropenylboronic acid pinacol ester (220.1 mg) were added thereto, after nitrogen replacement for three times, it was raised to 90° C. and stirred to react for 4 hours. When the reaction monitored by LCMS was completed, the system was concentrated under reduced pressure, and a residue was purified by silica gel column chromatography to obtain the title compound. MS (ESI) m/z (M+H)⁺=371.1.

### Step 2: Preparation of 4-(methylthio)-2-phenyl-5-(propyl-1-ene-2-yl)thieno[2,3-d]pyrimidine-6-carboxylic acid

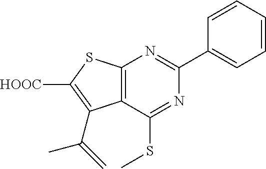 [SMILES: C=C(C)c1c(C(=O)O)sc2nc(-c3ccccc3)nc(SC)c12]

Ethyl 4-(methylthio)-2-phenyl-5-(propyl-1-ene-2-yl)thieno[2,3-d]pyrimidine-6-carboxylate (160 mg) was dissolved in a mixed solution of ethanol and water, then lithium hydroxide monohydrate (90 mg) was added thereto, and it was raised to 100° C. by heating and stirred to react for 1 hour. When the reaction monitored by LCMS was completed, the reaction was stopped. The system was directly concentrated under reduced pressure, and a residue was purified by reversed phase column chromatography to obtain the title compound. MS (ESI) m/z (M+H)⁺=343.1.

### Step 3: Preparation of (4-(methylthio)-2-phenyl-5-(propyl-1-ene-2-yl)thieno[2,3-d]pyrimidin-6-yl)(piperidin-1-yl)methanone

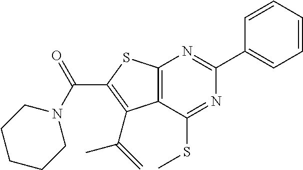 [SMILES: C=C(C)c1c(C(=O)N2CCCCC2)sc2nc(-c3ccccc3)nc(SC)c12]

4-(methylthio)-2-phenyl-5-(propyl-1-ene-2-yl)thieno[2,3-d]pyrimidine-6-carboxylic acid (200 mg) was dissolved in N,N-dimethylformamide (12 mL), then 2-(7-azabenzotriazol-1-yl)-N,N,N′,N′-tetramethyluronium hexafluorophosphate (441.1 mg), N,N-diisopropylethylamine (149.9 mg), and piperidine (98.8 mg) were added thereto, and is was stirred at room temperature to react overnight. When LC-MS showed that the reaction of raw materials was completed, the system was concentrated under reduced pressure, and a residue was purified by silica gel column chromatography to obtain the title compound. MS (ESI) m/z (M+H)⁺=410.1.

### Step 4: Preparation of (4-amino-2-phenyl-5-(propyl-1-ene-2-yl)thieno[2,3-d]pyrimidin-6-yl)(piperidin-1-yl)methanone

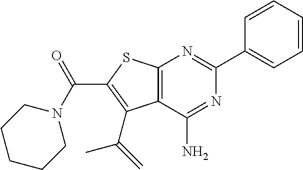 [SMILES: C=C(C)c1c(C(=O)N2CCCCC2)sc2nc(-c3ccccc3)nc(N)c12]

(4-(methylthio)-2-phenyl-5-(propyl-1-ene-2-yl)thieno[2,3-d]pyrimidin-6-yl)(piperidin-1-yl)methanone (120 mg) was dissolved in N-methylpyrrolidone (2 mL), aqueous ammonia (4 mL) was added thereto, and it was stirred under microwave at 150° C. to react for 6 hours. When LC-MS showed that most of raw materials depleted, the reaction was stopped. The system was concentrated under reduced pressure, and a residue was purified by silica gel column chromatography to obtain the title compound. MS (ESI) m/z (M+H)⁺=379.2.

### Step 5: Preparation of (4-amino-2-phenyl-5-isopropylthieno[2,3-d]pyrimidin-6-yl)(piperidin-1-yl)methanone

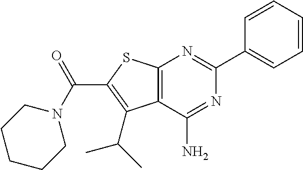 [SMILES: CC(C)c1c(C(=O)N2CCCCC2)sc2nc(-c3ccccc3)nc(N)c12]

(4-amino-2-phenyl-5-(propyl-1-ene-2-yl)thieno[2,3-d]pyrimidin-6-yl)(piperidin-1-yl)methanone (100 mg) was dissolved in tetrahydrofuran (10 mL), palladium on carbon (28.1 mg) was added thereto, and it was stirred at room temperature to react for 10 hours. When LC-MS showed that the reaction was completed, the reaction was stopped. The system was concentrated under reduced pressure, and a residue was purified by silica gel column chromatography to obtain the title compound. MS (ESI) m/z (M+H)⁺=381.2.

### Step 6: Preparation of (9-isopropyl-5-phenylimidazo[1,2-c]thieno[3,2-e]pyrimidin-8-yl)(piperidin-1-yl)methanone

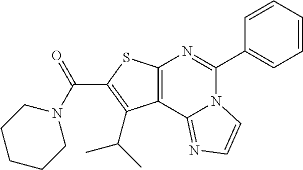 [SMILES: CC(C)c1c(C(=O)N2CCCCC2)sc2nc(-c3ccccc3)n3ccnc3c12]

(4-amino-2-phenyl-5-isopropylthieno[2,3-d]pyrimidin-6-yl)(piperidin-1-yl)methanone (30 mg) was dissolved in acetonitrile (4 mL), 2-bromo-1,1-diethoxyethane (67.1 mg) was added thereto, and then it was raised to 120° C. via microwave and stirred to react for 3 hours. When LC-MS showed that raw materials depleted, the reaction was stopped. The system was concentrated under reduced pressure, and a residue was purified by Prep-HPLC to obtain the title compound. MS (ESI) m/z (M+H)⁺=405.2.

¹H NMR (400 MHz, DMSO-d₆) δ 7.99 (d, J=1.6 Hz, 1H), 7.98-7.93 (m, 2H), 7.71 (d, J=1.6 Hz, 1H), 7.70-7.62 (m, 3H), 3.88-3.81 (m, 1H), 3.65 (s, 2H), 3.37 (s, 2H), 1.64 (d, J=5.7 Hz, 2H), 1.56 (s, 4H), 1.50 (d, J=7.0 Hz, 6H).

### Example 24: Preparation of 5-(9-amino-8-(piperidine-1-carbonyl)imidazo[1,2-c]thieno[3,2-e]pyrimidin-5-yl)-2-methylisoindol-1-one

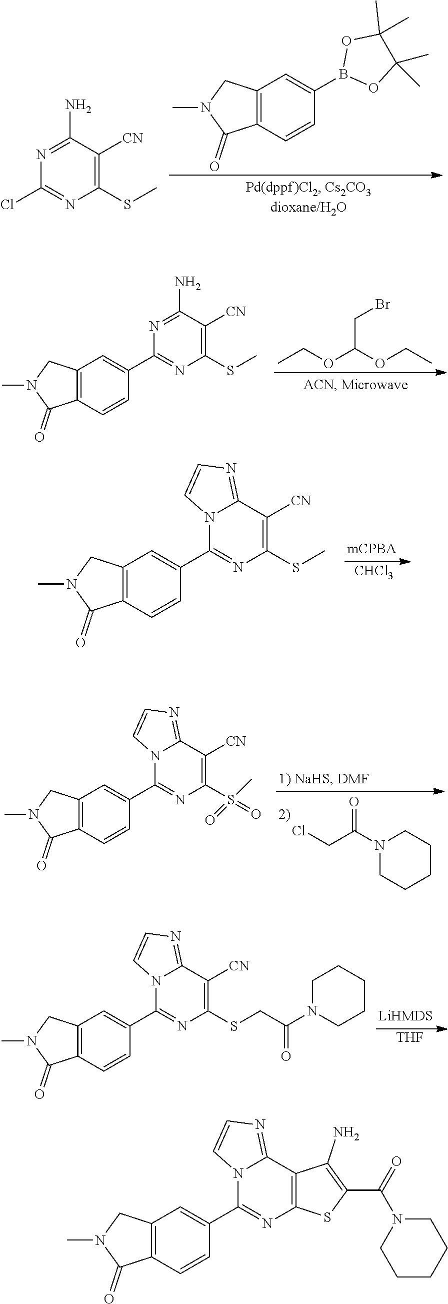

### Step 1: Preparation of 4-amino-2-(2-methyl-1-oxoisoindolin-5-yl)-6-(methylthio)pyrimidine-5-carbonitrile

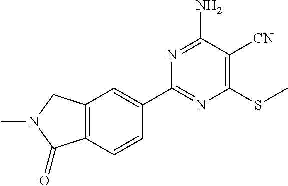

4-amino-2-chloro-6-(methylthio)pyrimidine-5-carbonitrile (1.50 g) and 2-methyl-5-(4,4,5,5-tetramethyl-1,3,2-dioxaborolan-2-yl)isoindolin-1-one (2.04 g) were added to 1,4-dioxane (20 mL) and water (2.0 mL). Cesium carbonate (3.65 g) and [1,1′-bis(diphenylphosphino)ferrocene]dichloropalladium (610 mg) were added thereto under the protection of nitrogen. A reaction was carried out under stirring at 100° C. for 12 hours. When LCMS showed that the reaction was completed, dimethyl sulfoxide was added into the reaction solution, and the reaction solution was stirred at 40° C. for 0.5 hours, then fully dissolved and filtered. 200 mL of water was added to the filtrate, and it was stirred for 0.5 hour until a solid was precipitated. A mixture was filtered, a filter cake was rinsed with water (50 mL*2), and a solid was dried to obtain a crude product. The crude product was separated and purified again by prep-HPLC to obtain the title compound. MS (ESI) m/z (M+H)⁺=312.2.

### Step 2: Preparation of 5-(2-methyl-1-oxoisoindolin-5-yl)-7-(methylthio)imidazo[1,2-c]pyrimidine-8-carbonitrile

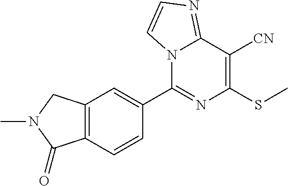

4-amino-2-(2-methyl-1-oxoisoindolin-5-yl)-6-(methylthio)pyrimidine-5-carbonitrile (0.5 g) and 2-bromo-1,1-diethoxyethane (2 mL) were added to a microwave tube. Acetonitrile (10 mL) was added to the microwave tube. A reaction was carried out under microwave irradiation for 1.5 hours. TLC and LCMS showed that the reaction was completed. A reaction solution was adjusted to weakly alkaline with a saturated sodium bicarbonate solution, and extracted with DCM and MeOH at a ratio of 10:1 (V/V) for several times. Organic phases were combined, dried over anhydrous sodium sulfate, concentrated, and purified by a silica gel column to obtain the title compound. MS (ESI) m/z (M+H)⁺=336.1.

### Step 3: Preparation of 7-methylsulfonyl-5-(2-methyl-1-oxo-2,3-dihydro-1H-isoindol-5-yl)imidazo[1,2-c]pyrimidine-8-carbonitrile

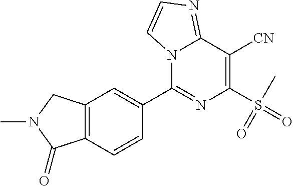 [SMILES: CN1Cc2cc(-c3nc(S(C)(=O)=O)c(C#N)c4nccn34)ccc2C1=O]

5-(2-methyl-1-oxoisoindolin-5-yl)-7-(methylthio)imidazo[1,2-c]pyrimidine-8-carbonitrile (0.15 g) was added to chloroform (5 mL). 3-chloroperoxybenzoic acid (300 mg) was added at 20° C. A reaction solution was stirred at 45° C. for 3 hours. When LCMS detected that a reaction was completed, the reaction solution was poured into a saturated sodium carbonate aqueous solution and extracted with dichloromethane, and an organic phase was dried, concentrated, and purified by a silica gel column to obtain the title compound (70 mg). MS (ESI) m/z (M+H)⁺=368.0.

### Step 4: Preparation of 5-(2-methyl-1-oxoisoindol-5-yl)-7-((2-oxo-2-(piperidin-1-yl)ethyl)thio)imidazo[1,2-c]pyrimidine-8-carbonitrile

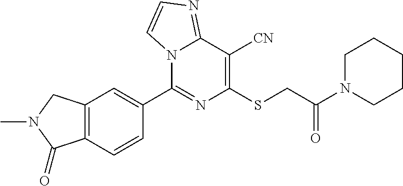 [SMILES: CN1Cc2cc(-c3nc(SCC(=O)N4CCCCC4)c(C#N)c4nccn34)ccc2C1=O]

7-methylsulfonyl-5-(2-methyl-1-oxo-2,3-dihydro-1H-isoindol-5-yl)imidazo[1,2-c]pyrimidine-8-carbonitrile (100 mg) was dissolved in DMF, sodium hydrosulfide (43.25 mg, purity: 70%) was added to carry out a reaction at 50° C. for 30 minutes, and the reaction monitored by LCMS was completed. 2-chloro-1-(piperidin-1-yl)ethane-1-one (87.28 mg) was added to carry out a reaction continuously for 1 hour, and the reaction monitored by LCMS was completed. After cooling to room temperature, water was added for dilution, and extraction was performed with ethyl acetate. Organic phases were combined, dried over anhydrous sodium sulfate, concentrated, and purified by a silica gel column to obtain the title compound. MS (ESI) m/z (M+H)⁺=447.0.

### Step 5: Preparation of 5-(9-amino-8-(piperidin-1-carbonyl)imidazo[1,2-c]thieno[3,2-e]pyrimidin-5-yl)-2-methylisoindol-1-one

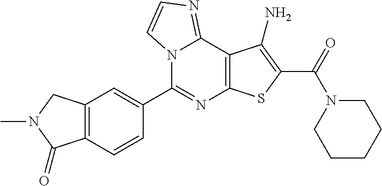 [SMILES: CN1Cc2cc(-c3nc4sc(C(=O)N5CCCCC5)c(N)c4c4nccn34)ccc2C1=O]

5-(2-methyl-1-oxoisoindol-5-yl)-7-((2-oxo-2-(piperidin-1-yl)ethyl)thio)imidazo[1,2-c]pyrimidine-8-carbonitrile (35 mg) was dissolved in dry tetrahydrofuran, lithium bis(trimethylsilyl)amide (0.2 mL, 1 M) was added under an ice water bath, a reaction was carried out at room temperature for 4 hours, and the reaction monitored by LCMS and TLC was completed. Quenching was performed, followed by extraction with ethyl acetate, drying, and separation by a reversed phase preparative column to obtain the title compound. MS(ESI) [M+H]⁺=447.1.

¹H NMR (400 MHz, DMSO-d₆) δ 8.17 (s, 1H), 8.07-8.04 (m, 2H), 7.91-7.89 (d, J=7.9 Hz, 1H), 7.70 (d, J=1.6 Hz, 1H), 6.48 (s, 2H), 4.61 (s, 2H), 3.63-3.60 (m, 4H), 3.14 (s, 3H), 1.64-1.58 (m, 6H).

### Example 25: Preparation of 6-(9-amino-8-(piperidin-1-carbonyl)imidazo[1,2-c]thieno[3,2-e]pyrimidin-5-yl)-2-methylisoquinolin-1(2H)-one

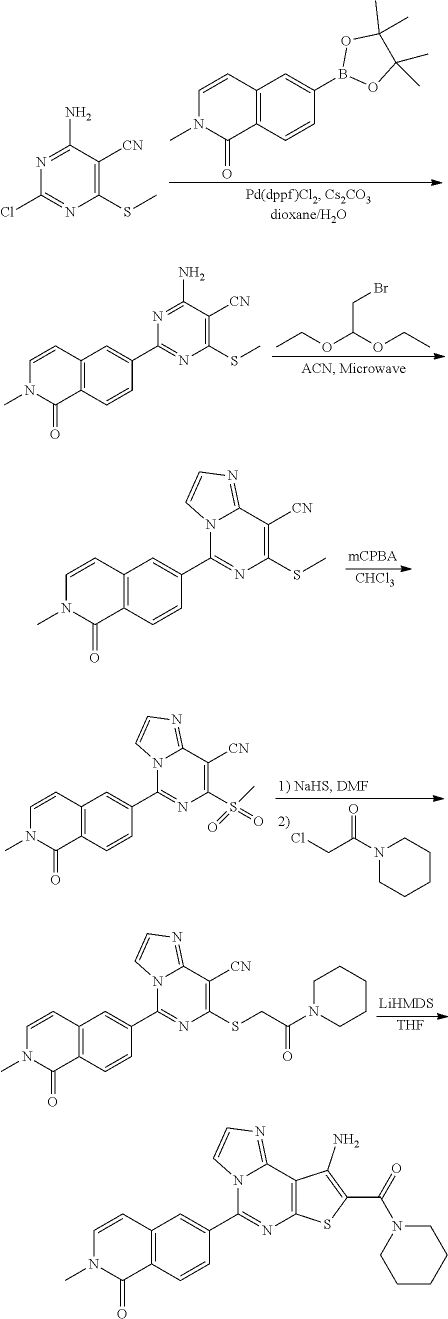

The title compound was prepared with reference to the method similar to that of Example 24.

MS(ESI) [M+H]⁺=459.1.¹H NMR (400 MHz, DMSO-d₆) δ 8.44-8.42 (d, J=8.4 Hz, 1H), 8.29 (d, J=1.7 Hz, 1H), 8.09 (d, J=1.7 Hz, 1H), 8.03-8.00 (dd, J=8.4, 1.8 Hz, 1H), 7.71 (d, J=1.6 Hz, 1H), 7.63-7.60 (d, J=7.3 Hz, 1H), 6.81 (d, J=7.4 Hz, 1H), 6.49 (s, 2H), 3.63-3.60 (m, 4H), 3.57 (s, 3H), 1.65-1.58 (m, 6H).

### Biological Tests

### Test Example 1: Detection of Activity of 15-PGDH Kinase

### 1. Experimental Materials:

| Reagents/Materials/Instruments | Manufacturer | Item No./Model No. |
|---|---|---|
| 15-PGDH | Sino Biological Inc. | 11205-H08E |
| β-NAD | Sigma-Aldrich Corporation | N6522 |
| PGF2α | MedChemExpress LLC | HY-12956A |
| DMSO | Sigma-Aldrich Corporation | D8418 |
| 384-well plate | Corning United States Corporation | 4513 |
| Tween 20 | Shanghai Macklin Biochemical | T818927 |
| | Technology Co., Ltd | |
| Tris-HCl | Shanghai Beyotime Biotech Inc. | ST774 |
| Multifunctional microplate reader | BMG LABTECH Corporation | PHERAstar ® FSX |

### 2. Experimental Method:

a. A solution of pH 7.5 containing 50 mM Tris-HCl, 0.01% Tween 20 was prepared with ultrapure water as a reaction buffer;
b. A 10 mM mother liquor of the compound to be tested was prepared with DMSO, and then the reaction buffer was used to dilute the mother liquor of the compound to be tested to obtain solution 1 of the compound to be tested at a concentration of 40,000 nM, and then the solution 1 of the compound to be tested was serially diluted into solutions 2-9 (or 2-12) of the compound to be tested at 9 (or 11) concentrations with a gradient difference of three-fold. 5 μL of each concentration of solutions of the compound to be tested was respectively taken and added into a 384-well plate as test wells;
c. 5 μL of the reaction buffer was added to the blank wells of the 384-well plate as positive control and blank control wells, respectively;
d. The reaction buffer was used to prepare a 15-PGDH protein solution at a concentration of 5 ng/μL, 5 μL of the 15-PGDH protein solution was taken and added to the test wells and positive control wells, and meanwhile 5 μL of the reaction buffer was added to the blank control wells, then the plate was centrifuged at 2000 rpm for 30 seconds;
e. The reaction buffer was used to prepare 5 mM β-NAD and 2 mM PGF2α, respectively, which were mixed at 1:1 by volume to obtain a substrate mixture, 10 μL of the substrate mixture was taken and added to the test wells, positive control wells and blank control wells to start the reaction;
f. The fluorescence signal value (Ex/Em=340/450) of each well was detected continuously by using a multifunctional microplate reader.

### 3. Data Analysis:

a) Continuous fluorescence signal values were analyzed by using the “kinetic calculations-slope calculation method” in the PHERAstar Data analysis software to obtain the slope of each test well;
b) The inhibition rate % was calculated by using the following formula:

$$inhibition rate \% = [ \text{} 1 - ( slope of test well - signal value of positive control well ) \text{} / ( signal value of blank control well - average signal value of positive control well ) ] \times 100 \% .$$

c) Calculation of IC50 and plotting of inhibition rate-dose curves: IC50 values were calculated by fitting compound concentrations and corresponding inhibition rates with a nonlinear regression (dose response-variable slope) via using GraphPad Prism 6.0. The formula was shown below:

Y=Bottom+(Top−Bottom)/(1+10\hat{ }((Log IC50−X)*HillSlope)), wherein X is a log value of a concentration of the compound, and Y is inhibition rate %.

4. Determination Results of the Compounds in the Examples of the Present Application were as Follows:

| Example No. | IC₅₀ (nM) |
|---|---|
| Example 1 | A |
| Example 2 | B |
| Example 3 | A |
| Example 4 | A |
| Example 5 | B |
| Example 6 | C |
| Example 7 | A |
| Example 8 | B |
| Compound 8 | C |
| Example 9 | B |
| Example 10 | B |
| Example 11 | B |
| Example 12 | B |
| Example 13 | B |
| Example 14 | B |
| Example 15 | A |
| Example 16 | D |
| Example 17 | D |
| Example 18 | C |
| Example 19 | C |
| Example 20 | C |
| Example 21 | A |
| Example 22 | B |
| Example 23 | B |
| Example 24 | A |
| Example 25 | A |

In the table, “A” represents that IC₅₀ of the inhibitory activity against 15-PGDH enzyme is in a range of less than 3 nM; “B” represents that IC₅₀ of the inhibitory activity against 15-PGDH enzyme is in a range of equal to or greater than 3 nM and less than 10 nM; “C” represents that IC₅₀ of the inhibitory activity against 15-PGDH enzyme is in a range of equal to or greater than 10 nM and less than 25 nM; “D” represents that IC₅₀ of the inhibitory activity against 15-PGDH enzyme is in a range of equal to or greater than 25 nM and less than 50 nM.

The compounds of the present application can exhibit inhibitory activity against 15-PGDH enzyme. The IC₅₀ value of the inhibitory activity of the compounds of the present application against 15-PGDH enzyme may be less than 100 nM, the IC₅₀ value of the inhibitory activity of some compounds in the present application against 15-PGDH enzyme is equal to or greater than 20 nM and less than 50 nM, the IC₅₀ value of the inhibitory activity of some compounds in the present application against 15-PGDH enzyme is equal to or greater than 10 nM and less than 20 nM, the IC₅₀ value of the inhibitory activity of some compounds in the present application against 15-PGDH enzyme is equal to or greater than 3 nM and less than 10 nM, the IC₅₀ value of the inhibitory activity of some compounds in the present application against 15-PGDH enzyme is equal to or greater than 1.5 nM and less than 3 nM, and the IC₅₀ value of the inhibitory activity of some compounds in the present application against 15-PGDH enzyme is less than 1.5 nM. For example, the IC₅₀ value of the inhibitory activity of the compound in Example 1 against 15-PGDH enzyme is less than 1.5 nM.

### Test Example 2: Assay of Intracellular PGE2 Up-Regulatory Activity

### 1. Experimental Materials:

| Reagents/Materials/Instruments | Manufacturer | Item No. |
|---|---|---|
| F12k Kaighn's Modification culture | Hyclone Laboratories, Inc | SH3052601/AG29722854 |
| medium | | |
| TRYPSIN | Hyclone Laboratories, Inc | J190002 |
| Fetal Bovine Serum | PAN-Biotech | ST-30-3302 |
| Penicillin-Streptomycin | Hyclone Laboratories, Inc | J190007 |
| DMSO | Sigma-Aldrich Corporation | D8418 |
| A549 cells | Cobioer Biosciences Co., LTD | CBP60084 |
| Prostaglandin E2 Kit | PerkinElmer Corporation | 62P2APEG |
| 24-well plate | Corning United States | 3337 |
| | Corporation | |
| 384-well plate | Corning United States | 3570 |
| | Corporation | |
| Multifunctional microplate reader | BMG LABTECH Corporation | PHERAstar ® FSX |
| CO₂ cell incubator | Thermo Fisher Scientific | RI-250 |
| | Corporation | |
| Microscope | Thermo Fisher Scientific | DMI1 |
| | Corporation | |

### 2. Experimental Method:

a) A549 cells were inoculated in the 24-well plate, and after cell adhesion, IL-1β was added thereto for 16 h of stimulation to induce COX2 expression and PGE2 production.
b) A solution of the compound to be tested was prepared with the culture medium and gradiently diluted to 3 concentrations of 10 nM, 300 nM and 10,000 nM or 7 concentrations of 0.64 nM, 3.2 nM, 16 nM, 80 nM, 400 nM, 2000 nM and 10,000 nM, and meanwhile the positive control group (only IL-1β was added to the cells for induction) and negative control group (only cells were added in the wells without any treatment) were set up. The cell supernatants were collected after 8 h of action, in which the positive control group was induced by IL-1β without treatment of the compounds, and the negative control group was neither stimulated by IL-1β, nor treated with the compounds.
c) The PGE2 content of the samples was determined by Prostaglandin E2 Kit, and the fluorescence signal was detected by a multifunctional microplate reader (Ex/Em=337/620, 337/665).

### 3. Data Analysis:

a) A standard curve was plotted with the PGE2 standard in the Prostaglandin E2 Kit, and the PGE2 concentration was calculated by substituting with the fluorescence signal of the sample.
b) The PGE2 up-regulation rate % was calculated by using the following formula:

$$PGE 2 = up - regulation rate \% = ( PGE 2 concentration of sample group / PGE 2 concentration of positive control group ) \times 100 \% .$$

### 4. Experimental Results

The compounds of the present application are able to achieve a PGE2 up-regulation rate of greater than 100% in A549 cells, some compounds of the present application are able to achieve a PGE2 up-regulation rate of greater than 200% in A549 cells, and some preferred compounds of the present application are able to achieve a PGE2 up-regulation rate of greater than 300% or higher in A549 cells. For example, the title compound of Example 8 has a PGE2 up-regulation rate of 483%, 520%, and 770% at the concentrations of 16 nM, 80 nM, and 400 nM in A549 cells, respectively; the title compound of Example 24 has a PGE2 up-regulation rate of 568%, 461%, and 473% at the concentrations of 3.2 nM, 16 nM, and 80 nM in A549 cells, respectively; the title compound of Example 25 has a PGE2 up-regulation rate of 513%, 782%, and 741% at the concentrations of 3.2 nM, 16 nM, and 80 nM in A549 cells, respectively; and the title compounds of Example 9 and Example 21 have a PGE2 up-regulation rate of 333% and 321% at the concentration of 400 nM in A549 cells, respectively. The compounds of the present application may have good intracellular PGE2 up-regulatory activities.

For the purpose of describing and disclosing, all patents, patent applications and other established publications are expressly incorporated herein by reference. These publications are provided solely for their disclosure prior to the filing date of this application. All statements regarding the dates of these documents or the representation of the contents of these documents are based on the information available to the applicants and do not constitute any admission as to the correctness of the dates of these documents or the contents of these documents. Moreover, any reference to these publications herein does not constitute an admission that the publications form part of the common general knowledge in the art in any country.

Those skilled in the art will recognize that the scope of the present application is not limited to the various specific embodiments and examples described above, but is capable of making various modifications, substitutions, or recombinations without departing from the spirit of the present application, and that these adjusted technical solutions fall within the protection scope of the present application.

1. A compound represented by formula (I), a stereoisomer, tautomer or mixture form thereof, or a pharmaceutically acceptable salt thereof, or a solvate thereof, or a prodrug thereof, 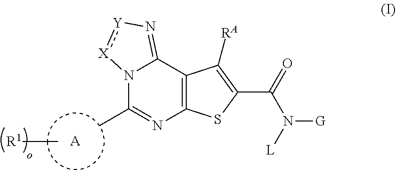
ring A is an aromatic ring, an aromatic heterocycle, or an unsaturated aliphatic heterocycle;
L and G are each independently a C₁-C₁₀ chain hydrocarbon group, or L, G, and an N atom connected thereto together form a cyclic group, and the cyclic group is a 3- to 12-membered saturated aliphatic heterocycle or a ring formed by a 3- to 12-membered saturated aliphatic heterocycle fused with a benzene ring;
o is 0, 1, 2, or 3;
R¹ is each independently selected from the group consisting of deuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₂-C₆ alkenyl, C₁-C₆ alkoxy, and a 3- to 8-membered saturated aliphatic heterocycle, or when o is 2 or 3, any two R¹ and atoms of ring A connected thereto together form a 3- to 8-membered alicyclic group or a 3- to 8-membered aliphatic heterocyclic group; [symbol]
is a single bond or a double bond, and when [symbol] is a double bond, X and Y are each independently CR^{B} or N; when [symbol] is a singe bond, X and Y are CR^{C}R^{D};
R^{A}, R^{B}, R^{C}, and R^{D} are each independently selected from the group consisting of hydrogen, hydroxyl, halogen, an amine group, cyano, C₃-C₈ cycloalkyl, C₁-C₆ alkyl, C₁-C₆ alkoxy, and C₁-C₆ haloalkyl;
wherein the aromatic heterocycle, the saturated aliphatic heterocycle, the unsaturated aliphatic heterocycle, and the aliphatic heterocyclic group each independently include 1-3 heteroatoms which are independently selected from the group consisting of N, O, and S, and the saturated aliphatic heterocycle includes at least 1 nitrogen atom;
the L, G, and R¹ are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6- to 10-membered aryl, and 5- to 10-membered heteroaryl.

2. The compound, the stereoisomer, tautomer or mixture form thereof, or the pharmaceutically acceptable salt thereof, or the solvate thereof, or the prodrug thereof according to claim 1, wherein the R¹ is each independently selected from the group consisting of deuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₁-C₆ alkoxy, and a 3- to 8-membered saturated aliphatic heterocyclic group, or when o is 2 or 3, any two R¹ and atoms of ring A connected thereto together form a 3- to 8-membered alicyclic group or a 3- to 8-membered aliphatic heterocyclic group, wherein the R¹ is optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6- to 10-membered aryl, and 5- to 10-membered heteroaryl.

3. The compound, the stereoisomer, tautomer or mixture form thereof, or the pharmaceutically acceptable salt thereof, or the solvate thereof, or the prodrug thereof according to claim 1, wherein the L and G are each independently C₁-C₁₀ alkyl, and the L and G are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6- to 10-membered aryl, and 5- to 10-membered heteroaryl; or
L and G are each independently selected from the group consisting of methyl, ethyl, n-propyl, isopropyl, n-butyl, isobutyl, tert-butyl, hydroxymethyl, hydroxyethyl, and hydroxypropyl.

4. The compound, the stereoisomer, tautomer or mixture form thereof, or the pharmaceutically acceptable salt thereof, or the solvate thereof, or the prodrug thereof according to claim 1, wherein the compound is represented by formula (II): 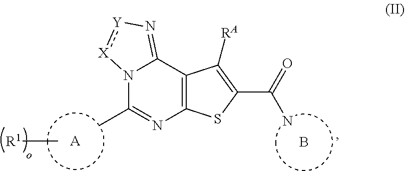
ring B is a 3- to 12-membered saturated aliphatic heterocycle or a ring formed by a 3- to 12-membered saturated aliphatic heterocycle fused with a benzene ring;
wherein the aromatic heterocycle, the saturated aliphatic heterocycle, the unsaturated aliphatic heterocycle, and the aliphatic heterocycle group each independently include 1-3 heteroatoms, the heteroatom is independently selected from the group consisting of N, O, and S, and ring B includes at least 1 nitrogen atom;
the ring B and R¹ are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6- to 10-membered aryl, and 5- to 10-membered heteroaryl;
the ring B is a monocycle, a fused ring, a bridged ring, or a spirocycle.

5. The compound, the stereoisomer, tautomer or mixture form thereof, or the pharmaceutically acceptable salt thereof, or the solvate thereof, or the prodrug thereof according to claim 4, wherein ring B is 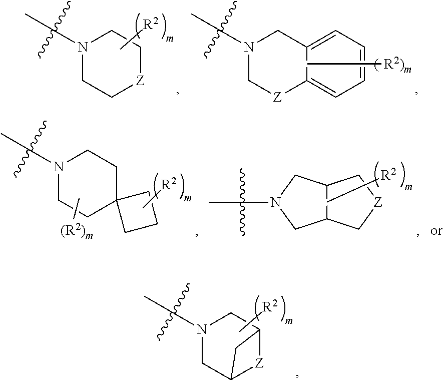
wherein Z is selected from the group consisting of a covalent bond, O, S, NH, (CH₂)ₙ, and SO₂; m is 0, 1, 2, or 3; R² is each independently selected from the group consisting of deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6- to 10-membered aryl, and 5- to 10-membered heteroaryl; the n is 1, 2, or 3; or the ring B is

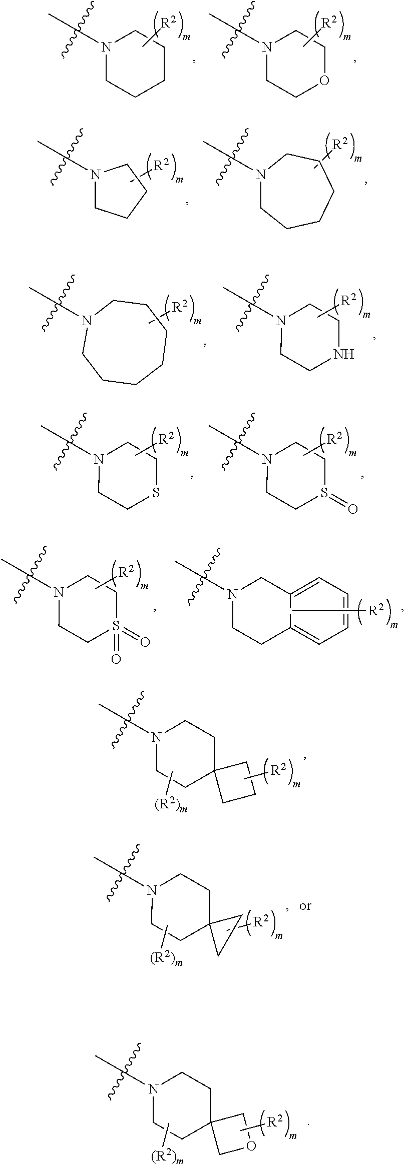

6. The compound, the stereoisomer, tautomer or mixture form thereof, or the pharmaceutically acceptable salt thereof, or the solvate thereof, or the prodrug thereof according to claim 5, wherein the R² is each independently selected from the group consisting of deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl.

7. The compound, the stereoisomer, tautomer or mixture form thereof, or the pharmaceutically acceptable salt thereof, or the solvate thereof, or the prodrug thereof according to claim 1, wherein the compound has a structure represented by formula III, 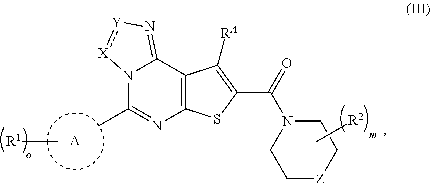
wherein Z is selected from the group consisting of a covalent bond, S, CH₂, (CH₂)₂, and (CH₂)₃; m is 0, 1, or 2; R² is each independently selected from the group consisting of deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6- to 10-membered aryl, and 5- to 10-membered heteroaryl; or
the R² is each independently selected from the group consisting of deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl.

8. The compound, the stereoisomer, tautomer or mixture form thereof, or the pharmaceutically acceptable salt thereof, or the solvate thereof, or the prodrug thereof according to claim 7, wherein the Z is a covalent bond or CH₂.

9. The compound, the stereoisomer, tautomer or mixture form thereof, or the pharmaceutically acceptable salt thereof, or the solvate thereof, or the prodrug thereof according to claim 1, wherein [symbol] is a single bond, the X and Y are CR^{C}R^{D}, and the R^{C} and R^{D} are independently selected from the group consisting of hydrogen, hydroxyl, cyano, halogen, methyl, ethyl, isopropyl, methoxy, ethoxy, trifluoromethyl, trifluoroethyl, trichloromethyl, trichloroethyl, cyclopropyl, cyclobutyl, and cyclopentyl.

10. The compound, the stereoisomer, tautomer or mixture form thereof, or the pharmaceutically acceptable salt thereof, or the solvate thereof, or the prodrug thereof according to claim 1, wherein [symbol] is a double bond, at least one of the X and Y is CR^{B}, and the R^{B} is selected from the group consisting of hydrogen, hydroxyl, cyano, halogen, C₃-C₈ cycloalkyl, C₁-C₆ alkyl, C₁-C₆ alkoxy, and C₁-C₆ haloalkyl; or both the X and Y are CR^{B}, and the R^{B} is selected from the group consisting of hydrogen, hydroxyl, cyano, halogen, C₃-C₈ cycloalkyl, C₁-C₆ alkyl, C₁-C₆ alkoxy, and C₁-C₆ haloalkyl; or the Y is N, the X is CR^{B}, and the R^{B} is selected from the group consisting of hydrogen, hydroxyl, cyano, halogen, C₃-C₈ cycloalkyl, C₁-C₆ alkyl, C₁-C₆ alkoxy, and C₁-C₆ haloalkyl; or the X is N, the Y is CR^{B}, and the R^{B} is selected from the group consisting of hydrogen, hydroxyl, cyano, halogen, C₃-C₈ cycloalkyl, C₁-C₆ alkyl, C₁-C₆ alkoxy, and C₁-C₆ haloalkyl.

11. The compound, the stereoisomer, tautomer or mixture form thereof, or the pharmaceutically acceptable salt thereof, or the solvate thereof, or the prodrug thereof according to claim 1, wherein the R^{A} is selected from the group consisting of hydrogen, hydroxyl, cyano, fluoro, chloro, bromo, iodo, —NH₂, methyl, ethyl, isopropyl, methoxy, ethoxy, trifluoromethyl, trifluoroethyl, trichloromethyl, trichloroethyl, cyclopropyl, cyclobutyl, and cyclopentyl.

12. The compound, the stereoisomer, tautomer or mixture form thereof, or the pharmaceutically acceptable salt thereof, or the solvate thereof, or the prodrug thereof according to claim 1, wherein the ring A is a 6- to 10-membered aromatic ring, a 5- to 10-membered aromatic heterocycle, or a 6- to 8-membered unsaturated aliphatic heterocycle; the aromatic ring and the aromatic heterocycle are a monocycle or a fused ring, the unsaturated aliphatic heterocycle is a monocycle, the aromatic heterocycle and the unsaturated aliphatic heterocycle each independently include 1-3 heteroatoms, and the heteroatom is independently selected from the group consisting of N, O, and S; or
the ring A is a benzene ring, a naphthalene ring, or a 5- to 10-membered aromatic heterocycle containing 1-2 heteroatoms, and the heteroatom is independently N or S; or the ring A is selected from the group consisting of

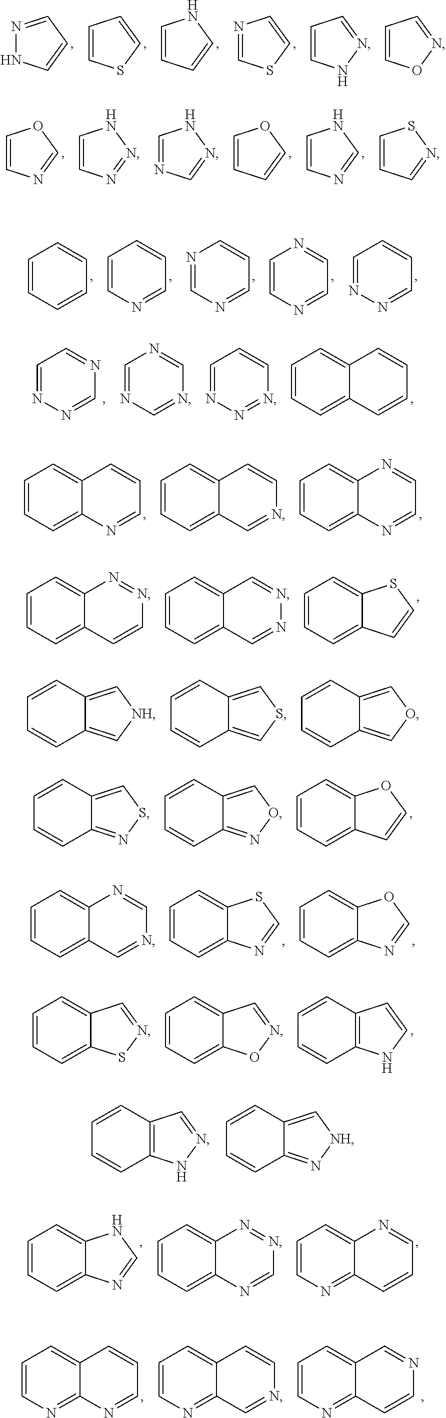

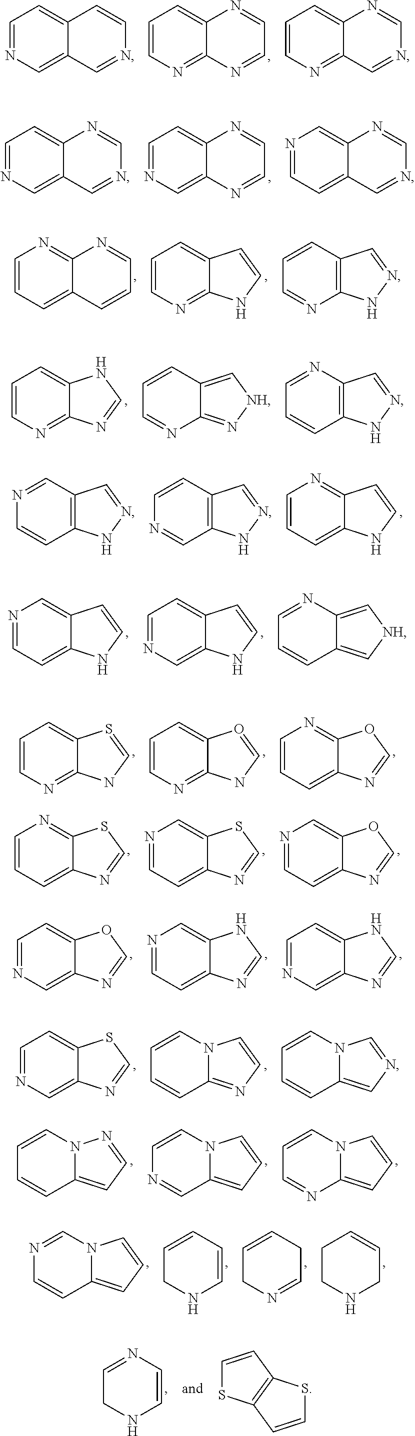

13. The compound, the stereoisomer, tautomer or mixture form thereof, or the pharmaceutically acceptable salt thereof, or the solvate thereof, or the prodrug thereof according to claim 1, wherein R¹ is each independently selected from the group consisting of deuterium, tritium, nitro, hydroxyl, mercapto, halogen, cyano, ═O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, tert-butyl, n-pentyl, isopentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, tert-butoxy, n-pentyloxy, isopentyloxy, tert-pentyloxy, and n-hexyloxy; or any two R¹ and atoms of ring A connected thereto together form dioxanyl, dioxolanyl, dioxenyl, dioxolyl, dihydropyridyl, or pyrrolinyl, wherein the R¹ is optionally substituted by one or more independently selected from the group consisting of deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6- to 10-membered aryl, and 5- to 10-membered heteroaryl.

14. The compound, the stereoisomer, tautomer or mixture form thereof, or the pharmaceutically acceptable salt thereof, or the solvate thereof, or the prodrug thereof according to claim 1, wherein R¹ is each independently selected from the group consisting of deuterium, tritium, nitro, hydroxyl, mercapto, halogen, cyano, ═O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, tert-butyl, n-pentyl, isopentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, tert-butoxy, n-pentyloxy, isopentyloxy, tert-pentyloxy, and n-hexyloxy; or any two R¹ and atoms of ring A connected thereto together form dioxanyl or dioxolanyl, wherein the R¹ is optionally substituted by one or more independently selected from the group consisting of deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6- to 10-membered aryl, and 5- to 10-membered heteroaryl.

15. The compound, the stereoisomer, tautomer or mixture form thereof, or the pharmaceutically acceptable salt thereof, or the solvate thereof, or the prodrug thereof according to claim 7, wherein the ring A is a benzene ring, a naphthalene ring, or a 5- to 10-membered aromatic heterocycle containing 1-2 heteroatoms, and the heteroatom is independently N or S;
o is 0 or 2, when o is 2, any two R¹ and atoms of ring A connected thereto together form saturated or unsaturated 5- to 6-membered cycloamido, and the cycloamido is optionally substituted by C₁-C₆ alkyl at the N atom therein;
the [symbol] is a single bond, the X and Y are CR^{C}R^{D}, and the R^{C} and R^{D} are each independently hydrogen, hydroxyl, or C₁-C₆ alkyl; or the [symbol] is a double bond, at least one of the X and Y is CR^{B}, and the R^{B} is hydrogen, C₃-C₈ cycloalkyl, C₁-C₆ alkyl, or C₁-C₆ haloalkyl;
R^{A} is selected from the group consisting of hydrogen, halogen, an amine group, cyano, C₃-C₆ cycloalkyl, C₁-C₆ alkyl, C₁-C₆ alkoxy, and C₁-C₆ haloalkyl;
the Z is a covalent bond or CH₂;
the m is 0, 1, or 2;
the R² is each independently fluoro, chloro, bromo, methyl, ethyl, n-propyl, or isopropyl.

16. The compound, the stereoisomer, tautomer or mixture form thereof, or the pharmaceutically acceptable salt thereof, or the solvate thereof, or the prodrug thereof according to claim 1, wherein the compound is:

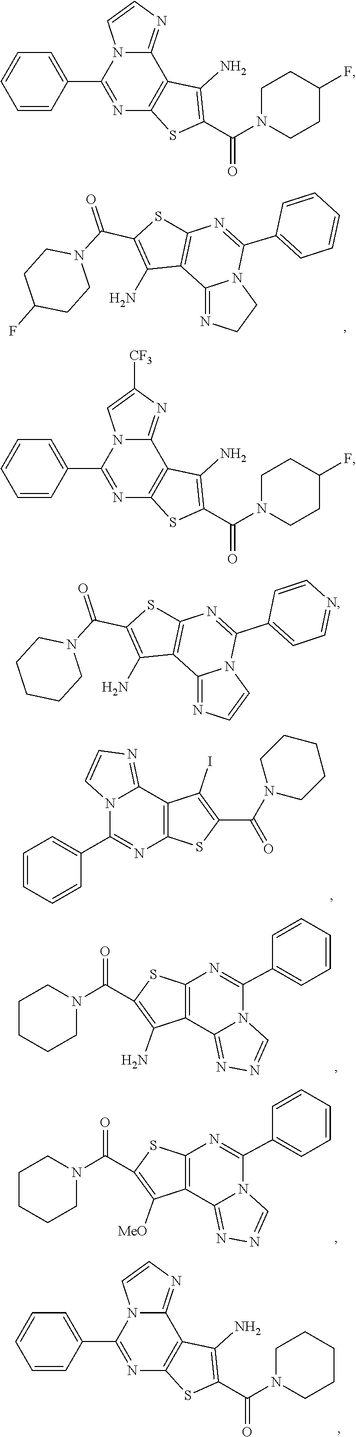

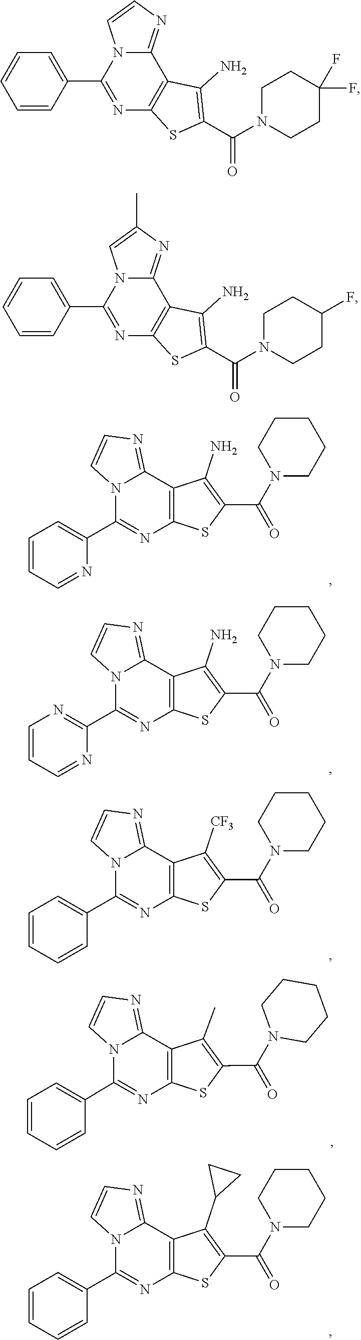

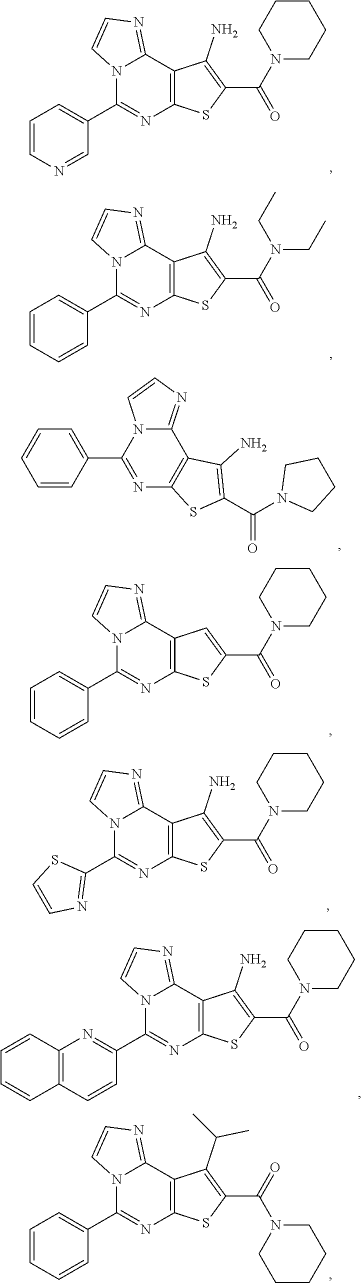

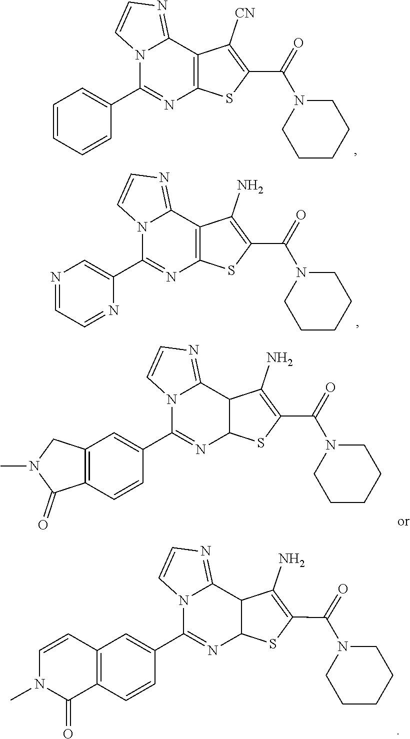

17. A pharmaceutical composition, comprising at least one of the compound, the tautomer or mixture form thereof, or the pharmaceutically acceptable salt thereof, or the solvate thereof, or the prodrug thereof according to claim 1, and at least one pharmaceutically acceptable excipient.

18. (canceled)

19. The compound, the stereoisomer, tautomer or mixture form thereof, or the pharmaceutically acceptable salt thereof, or the solvate thereof, or the prodrug thereof according to claim 10, wherein the R^{B} is selected from the group consisting of hydrogen, hydroxyl, cyano, fluoro, chloro, bromo, methyl, ethyl, isopropyl, methoxy, ethoxy, trifluoromethyl, trifluoroethyl, trichloromethyl, trichloroethyl, cyclopropyl, cyclobutyl, and cyclopentyl.

20. A method of treating or preventing a disease associated with 15-PGDH, comprising administering to a subject in need thereof the compound, the stereoisomer, tautomer or mixture form thereof, or the pharmaceutically acceptable salt thereof, or the solvate thereof, or the prodrug thereof according to claim 1, or a pharmaceutical composition thereof.

21. The method according to claim 20, wherein the compound, the stereoisomer, tautomer or mixture form thereof, or the pharmaceutically acceptable salt thereof, or the solvate thereof, or the prodrug thereof, or the pharmaceutical composition thereof is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, and underactive bladder, or for promoting hair growth, pigmentation, tissue repair, tissue regeneration, implant in stem cell transplantation or bone marrow transplantation or organ transplantation, neurogenesis and neuronal cell death, or muscle regeneration and cervical ripening, or for enhancing resistance to the toxicity of chemotherapy and the toxicity of immunosuppressant.
